(19) United States
(12) Patent Application Publication
(10) Pub. No.: US 2011/0104702 A1
(43) Pub. Date: May 5, 2011

(54) Methods for Predicting Tumor Response to Chemotherapy and Selection of Tumor Treatment

(75) Inventors: Soo-Chin Lee, Singapore (SG); Boon-Cher Goh, Singapore (SG)

(73) Assignee: NATIONAL UNIVERSITY HOSPITAL, Singapore (SG)

(21) Appl. No.: 13/000,108

(22) PCT Filed: Jul. 3, 2009
(86) PCT No.: PCT/SG2009/000241

§ 371 Date: Dec. 20, 2010

Related U.S. Application Data
(60) Provisional application No. 61/077,896, filed on Jul. 3, 2008.

(51) Int. Cl. C12Q 1/68

(52) U.S. Cl. 435/6

(57) ABSTRACT

Methods of selecting a treatment for further treatment of a tumor that has been exposed to chemotherapy and methods of predicting response of a tumor to chemotherapy are disclosed. Some of the methods involve performing gene expression analysis on a sample obtained from a patient having a tumor that has been exposed to chemotherapy so as to obtain a chemotherapy gene expression data set and analysing the chemotherapy gene expression data set to predict response of the tumor to the chemotherapy to which the tumor has been exposed.

### FIELD OF THE INVENTION

The present invention relates to methods for predicting the response of a tumor to chemotherapy and to selection of a treatment for further treatment of a tumor that has been exposed to chemotherapy.

### BACKGROUND TO THE INVENTION

Cancer is heterogeneous in biology. Therapeutic response and tumor gene expression signatures have been used to classify¹, prognosticate²⁻⁵ and predict chemotherapy sensitivity⁶⁻¹⁰. However, to date all efforts have been focused on the unchallenged baseline tumor.

Most cancer patients receive systemic chemotherapy, and exposure to drug may modify the tumor's short- and long-term outcomes. Drug-induced tumor gene signatures provide information on the tumor's responses to the drug and may provide insights into resistance mechanisms¹¹.

### SUMMARY OF THE INVENTION

The inventors have found that changes in tumor gene expression during chemotherapy provide gene expression signatures (chemotherapy induced gene expression signatures) that are superior at predicting tumor response to chemotherapy and relapse-free survival than gene expression signatures obtained from tumors prior to chemotherapy.

By analysing the gene expression signature of a tumor during chemotherapy a prognosis can be formed during the treatment. This may be a prediction of good or poor response to the applied chemotherapy. In the case of a predicted poor response, the chemotherapy treatment may be varied, changed or supplemented in an attempt to improve the treatment outcome. By accurately predicting tumor response to chemotherapy during treatment early intervention is enabled, particularly where a poor outcome is predicted.

Accordingly, the use of gene expression signatures obtained during chemotherapy to predict tumor response and enable selection of an alternative treatment strategy is provided. In some aspects and embodiments methods of selection of a treatment for further treatment of a tumor are provided. In some aspects and embodiments methods of predicting response of a tumor to chemotherapy are provided. Aspects and embodiments of the present invention also relate to methods of monitoring the effectiveness of a chemotherapy treatment to treat a tumor and/or the responsiveness of a tumor to a chemotherapy treatment.

Methods according to the present invention involve performing gene expression analysis on a sample obtained from a patient having a tumor that has been exposed to chemotherapy so as to obtain a chemotherapy gene expression data set and analysing the chemotherapy gene expression data to predict the response of the tumor to the chemotherapy treatment.

In some embodiments the chemotherapy gene expression data is compared with gene expression data obtained from the patient prior to exposure to chemotherapy. This comparison may be used to determine changes in the gene expression data between the untreated and chemotherapy treated tumor, which changes may be used to predict the tumor response and/or to select an alternative or supplementary treatment.

In other embodiments one or more further chemotherapy gene expression data sets is obtained after further chemotherapy treatment of the tumor. Comparison of the chemotherapy gene expression data obtained at different stages of the chemotherapy treatment may be used to determine changes in tumor gene expression as chemotherapy treatment progresses, thereby enabling effective monitoring of the tumor treatment, the predicted tumor response and treatment outcome. This also provides the ability to intervene early in the treatment and select an alternative or supplementary treatment.

In some embodiments gene expression data obtained from the tumor prior to and/or during chemotherapy is compared with gene expression data previously collected from other patients having a tumor(s) of corresponding type which have been treated with a corresponding chemotherapy and for which the tumor response (e.g. poor, good) to that chemotherapy is known. By comparing the gene expression data from the tumor undergoing treatment with gene expression data from patients having tumors of corresponding type that are known to be good or poor responders one can predict a good or poor response of the tumor subject to the ongoing chemotherapy.

In other embodiments the change in gene expression data between baseline tumor (i.e. prior to chemotherapy) and tumor during chemotherapy can be determined. This can be similarly compared with changes in gene expression data between baseline tumor and tumor during chemotherapy (preferably at a corresponding stage, e.g. number of cycles of treatment or time point in the chemotherapy) that has been previously collected from other patients having a tumor(s) of corresponding type which have been treated with a corresponding chemotherapy and for which the tumor response (e.g. poor, good) to that chemotherapy is known. By comparing the change in gene expression data from the tumor undergoing treatment with the change in gene expression data from patients having a tumor(s) of corresponding type that are known to be good or poor responders one can predict a good or poor response of the tumor subject to the ongoing chemotherapy.

In a similar embodiment the change in gene expression data obtained from the tumor at one stage or time point in the chemotherapy and one or more later stages or time points in the chemotherapy can be determined. This can be similarly compared with changes in gene expression data between corresponding stages or time points in chemotherapy previously collected from other patients having a tumor(s) of corresponding type which have been treated with a corresponding chemotherapy and for which the tumor response (e.g. poor, good) to that chemotherapy is known. By comparing the change(s) in gene expression data from the tumor undergoing treatment with the change(s) in gene expression data from patients having a tumor(s) of corresponding type that are known to be good or poor responders one can predict a good or poor response of the tumor subject to the ongoing chemotherapy.

In one aspect of the present invention a method of selecting a treatment for further treatment of a tumor that has been exposed to chemotherapy is provided, the method comprising:
performing gene expression analysis on a sample obtained from a patient having a tumor that has been exposed to chemotherapy so as to obtain a chemotherapy gene expression data set;
(ii) analysing said chemotherapy gene expression data set to predict response of the tumor to the chemotherapy;
(iii) based on said prediction selecting a treatment for further treatment of said tumor.

In some embodiments the step of analysing said chemotherapy gene expression data set comprises comparing gene expression data from said chemotherapy gene expression data set with gene expression data obtained for a corresponding tumor type which has been exposed to a corresponding chemotherapy and which gene expression data has been related with the response of the tumor type to the chemotherapy. In preferred embodiments a positive relationship of gene expression data between said gene expression data from said chemotherapy gene expression data set and gene expression data obtained for a corresponding tumor type is predictive of the response of the tumor being treated to the existing chemotherapy.

In one embodiment the step of analysing said chemotherapy gene expression data set comprises comparing gene expression data from said chemotherapy gene expression data set with gene expression data from corresponding tumor types known to be poor responders to the same chemotherapy. Preferably, positive relationship of gene expression data from said chemotherapy gene expression data set with gene expression data from corresponding tumor types known to be poor responders to the same chemotherapy is predictive of poor response of the tumor to the chemotherapy.

In one embodiment the step of analysing said chemotherapy gene expression data set comprises comparing gene expression data from said chemotherapy gene expression data set with gene expression data from corresponding tumor types known to be good responders to the same chemotherapy. Preferably, positive relationship of gene expression data from said chemotherapy gene expression data set with gene expression data from corresponding tumor types known to be good responders to the same chemotherapy is predictive of good response of the tumor to the chemotherapy.

In some embodiments the method comprises repeating the method one or more times, wherein in each repetition a chemotherapy gene expression data set obtained by performing gene expression analysis on a further sample obtained from the patient following further exposure of the tumor to the same or different chemotherapy is used.

In some embodiments the step of analysing said chemotherapy gene expression data to predict response of the tumor to the chemotherapy comprises comparing gene expression data from the chemotherapy gene expression data set with gene expression data from a further chemotherapy gene expression data set obtained by performing gene expression analysis on a further sample obtained from the patient following further exposure of the tumor to the same or different chemotherapy so as to determine a change in gene expression of the tumor, and analysing the change in gene expression to predict response of the tumor to the chemotherapy.

In another aspect of the present invention a method of selecting a treatment for further treatment of a tumor that has been exposed to chemotherapy is provided, the method comprising:
(a) performing gene expression analysis on a sample obtained from a patient having a tumor that has not been exposed to chemotherapy so as to obtain a baseline tumor gene expression data set;
(b) comparing gene expression data from said baseline tumor gene expression data set with gene expression data from a chemotherapy gene expression data set obtained by performing gene expression analysis on a sample obtained from the patient after exposure of the tumor to chemotherapy so as to determine a change in gene expression;
(c) analysing the change in gene expression to predict response of the tumor to the chemotherapy;
(d) based on said prediction selecting a treatment for further treatment of said tumor.

In some embodiments the step of analysing the change in gene expression to predict response of the tumor to the chemotherapy comprises comparing change(s) in gene expression from (b) with change(s) in baseline and chemotherapy gene expression obtained for a corresponding tumor type which has been exposed to a corresponding chemotherapy and which changes have been related with the response of the tumor type to the chemotherapy. In preferred embodiments a positive relationship of change(s) between said change in gene expression from (b) and change(s) in baseline and chemotherapy gene expression obtained for a corresponding tumor type is predictive of the response of the tumor being treated to the existing chemotherapy.

In one embodiment the step of analysing the change in gene expression to predict response of the tumor to the chemotherapy comprises comparing change(s) in gene expression from (b) with change(s) in baseline and chemotherapy gene expression from corresponding tumor types known to be poor responders to the same chemotherapy.

Preferably, positive relationship of change(s) in gene expression from (b) with change(s) in baseline and chemotherapy gene expression from corresponding tumor types known to be poor responders to the same chemotherapy is predictive of poor response of the tumor to the chemotherapy.

In one embodiment the step of analysing the change in gene expression to predict response of the tumor to the chemotherapy comprises comparing change(s) in gene expression from (b) with change(s) in baseline and chemotherapy gene expression from corresponding tumor types known to be good responders to the same chemotherapy. Preferably, positive relationship of change(s) in gene expression from (b) with change(s) in baseline and chemotherapy gene expression from corresponding tumor types known to be good responders to the same chemotherapy is predictive of good response of the tumor to the chemotherapy.

In some embodiments the method comprises repeating the method one or more times in which the baseline tumor gene expression data is compared with gene expression data from a further chemotherapy gene expression data set obtained by performing gene expression analysis on a further sample obtained from the patient following further exposure of the tumor to the same or different chemotherapy.

In another aspect of the present invention a method of selecting a treatment for further treatment of a tumor that has been exposed to chemotherapy is provided, the method comprising:
I. performing gene expression analysis on a sample obtained from a patient having a tumor that has been exposed to chemotherapy so as to obtain a first chemotherapy gene expression data set;
II. comparing gene expression data from said first chemotherapy gene expression data set with gene expression data from a second chemotherapy gene expression data set obtained by performing gene expression analysis on a sample obtained from the patient after further exposure of the tumor to the chemotherapy so as to determine a change in gene expression;
III. analysing the change in gene expression to predict response of the tumor to the chemotherapy;
IV. based on said prediction selecting a treatment for further treatment of said tumor.

In some embodiments the step of analysing the change in gene expression to predict response of the tumor to the chemotherapy comprises comparing change(s) in gene expression from II. with change(s) in first and second chemotherapy gene expression data sets obtained for a corresponding tumor type which has been exposed to a corresponding chemotherapy and which changes have been related with the response of the tumor type to the chemotherapy. In preferred embodiments a positive relationship of change(s) between said change in gene expression from II. and change(s) in first and second chemotherapy gene expression data sets obtained for a corresponding tumor type is predictive of the response of the tumor being treated to the existing chemotherapy.

In one embodiment the step of analysing the change in gene expression to predict response of the tumor to the chemotherapy comprises comparing change(s) in gene expression from II. with change(s) in first and second chemotherapy gene expression data sets from corresponding tumor types known to be poor responders to the same chemotherapy. Preferably, positive relationship of change(s) in gene expression from II. with change(s) in first and second chemotherapy gene expression data sets from corresponding tumor types known to be poor responders to the same chemotherapy is predictive of poor response of the tumor to the chemotherapy.

In one embodiment the step of analysing the change in gene expression to predict response of the tumor to the chemotherapy comprises comparing change(s) in gene expression from II. with change(s) in first and second chemotherapy gene expression data sets from corresponding tumor types known to be good responders to the same chemotherapy. Preferably, positive relationship of change(s) in gene expression from II. with change(s) in first and second chemotherapy gene expression data sets from corresponding tumor types known to be good responders to the same chemotherapy is predictive of good response of the tumor to the chemotherapy.

In some embodiments the step of selecting a treatment for further treatment of said tumor comprises selection of one or more of:
maintenance of the existing chemotherapy or treatment regime;
discontinuing the existing chemotherapy or treatment regime;
starting a new chemotherapy or treatment regime;
maintenance of the existing chemotherapy but altering the existing treatment regime.

In some embodiments, on the basis of a prediction of poor response to chemotherapy the step of selecting a treatment for further treatment of said tumor comprises one or more of:
discontinuing the existing chemotherapy or treatment regime;
starting a new chemotherapy or treatment regime;
maintenance of the existing chemotherapy but altering the existing treatment regime.

In some embodiments, on the basis of a prediction of good response to chemotherapy the step of selecting a treatment for further treatment of said tumor comprises one or more of:
maintenance of the existing chemotherapy or treatment regime;
maintenance of the existing chemotherapy but altering the existing treatment regime.

In a further aspect of the present invention a method of predicting response of a tumor to chemotherapy is provided, the method comprising:
(i) performing gene expression analysis on a sample obtained from a patient having a tumor that has been exposed to chemotherapy so as to obtain a chemotherapy gene expression data set;
(ii) analysing said chemotherapy gene expression data set to predict response of the tumor to the chemotherapy to which the tumor has been exposed.

In some embodiments the step of analysing said chemotherapy gene expression data set to predict response of the tumor to the chemotherapy to which the tumor has been exposed comprises comparing gene expression data from said chemotherapy gene expression data set with gene expression data obtained for a corresponding tumor type which has been exposed to a corresponding chemotherapy and which gene expression data has been related with the response of the tumor type to the chemotherapy. In preferred embodiments a positive relationship of gene expression data between said gene expression data from said chemotherapy gene expression data set and gene expression data obtained for a corresponding tumor type is predictive of the response of the tumor being treated to the existing chemotherapy.

In one embodiment the step of analysing said chemotherapy gene expression data set comprises comparing gene expression data from said chemotherapy gene expression data set with gene expression data from corresponding tumor types known to be poor responders to the same chemotherapy. Preferably, positive relationship of gene expression data from said chemotherapy gene expression data set with gene expression data from corresponding tumor types known to be poor responders to the same chemotherapy is predictive of poor response of the tumor to the chemotherapy.

In one embodiment the step of analysing said chemotherapy gene expression data set comprises comparing gene expression data from said chemotherapy gene expression data set with gene expression data from corresponding tumor types known to be good responders to the same chemotherapy. Preferably, positive relationship of gene expression data from said chemotherapy gene expression data set with gene expression data from corresponding tumor types known to be good responders to the same chemotherapy is predictive of good response of the tumor to the chemotherapy.

In some embodiments the method comprises repeating the method one or more times, wherein in each repetition a chemotherapy gene expression data set obtained by performing gene expression analysis on a further sample obtained from the patient following further exposure of the tumor to the same or different chemotherapy is used.

In some embodiments the step of analysing said chemotherapy gene expression data set to predict response of the tumor to the chemotherapy comprises comparing gene expression data from the chemotherapy gene expression data set with gene expression data from a further chemotherapy gene expression data set obtained by performing gene expression analysis on a further sample obtained from the patient following further exposure of the tumor to the same or different chemotherapy so as to determine a change in gene expression of the tumor, and analysing the change in gene expression to predict response of the tumor to the chemotherapy.

In yet a further aspect of the present invention a method of predicting response of a tumor to chemotherapy is provided, the method comprising:
(a) performing gene expression analysis on a sample obtained from a patient having a tumor that has not been exposed to chemotherapy so as to obtain a baseline tumor gene expression data set;
(b) comparing gene expression data from said baseline tumor gene expression data set with gene expression data from a chemotherapy gene expression data set obtained by performing gene expression analysis on a sample obtained from the patient after exposure of the tumor to chemotherapy so as to determine a change in gene expression;
(c) analysing the change in gene expression to predict response of the tumor to the chemotherapy.

In some embodiments the step of analysing the change in gene expression to predict response of the tumor to the chemotherapy comprises comparing change(s) in gene expression from (b) with change(s) in baseline and chemotherapy gene expression obtained for a corresponding tumor type which has been exposed to a corresponding chemotherapy and which changes have been related with the response of the tumor type to the chemotherapy. In preferred embodiments a positive relationship of change(s) between said change in gene expression from (b) and change(s) in baseline and chemotherapy gene expression obtained for a corresponding tumor type is predictive of the response of the tumor being treated to the existing chemotherapy.

In one embodiment the step of analysing the change in gene expression to predict response of the tumor to the chemotherapy comprises comparing change(s) in gene expression from (b) with change(s) in baseline and chemotherapy gene expression from corresponding tumor types known to be poor responders to the same chemotherapy. Preferably, positive relationship of change(s) in gene expression from (b) with change(s) in baseline and chemotherapy gene expression from corresponding tumor types known to be poor responders to the same chemotherapy is predictive of poor response of the tumor to the chemotherapy.

In one embodiment the step of analysing the change in gene expression to predict response of the tumor to the chemotherapy comprises comparing change(s) in gene expression from (b) with change(s) in baseline and chemotherapy gene expression from corresponding tumor types known to be good responders to the same chemotherapy. Preferably, positive relationship of change(s) in gene expression from (b) with change(s) in baseline and chemotherapy gene expression from corresponding tumor types known to be good responders to the same chemotherapy is predictive of good response of the tumor to the chemotherapy.

In some embodiments the method comprises repeating the method one or more times in which the baseline tumor gene expression data is compared with gene expression data from a further chemotherapy gene expression data set obtained by performing gene expression analysis on a further sample obtained from the patient following further exposure of the tumor to the same or different chemotherapy.

In yet a further aspect of the present invention a method of predicting response of a tumor to chemotherapy is provided, the method comprising:
I. performing gene expression analysis on a sample obtained from a patient having a tumor that has been exposed to chemotherapy so as to obtain a first chemotherapy gene expression data set;
II. comparing gene expression data from said first chemotherapy gene expression data set with gene expression data from a second chemotherapy gene expression data set obtained by performing gene expression analysis on a sample obtained from the patient after further exposure of the tumor to the chemotherapy so as to determine a change in gene expression;
III. analysing the change in gene expression to predict response of the tumor to the chemotherapy.

In some embodiments the step of analysing the change in gene expression to predict response of the tumor to the chemotherapy comprises comparing change(s) in gene expression from II. with change(s) in first and second chemotherapy gene expression data sets obtained for a corresponding tumor type which has been exposed to a corresponding chemotherapy and which changes have been related with the response of the tumor type to the chemotherapy. In preferred embodiments a positive relationship of change(s) between said change in gene expression from II. and change(s) in first and second chemotherapy gene expression data sets obtained for a corresponding tumor type is predictive of the response of the tumor being treated to the existing chemotherapy.

In one embodiment the step of analysing the change in gene expression to predict response of the tumor to the chemotherapy comprises comparing change(s) in gene expression from II. with change(s) in first and second chemotherapy gene expression data sets from corresponding tumor types known to be poor responders to the same chemotherapy. Preferably, positive relationship of change(s) in gene expression from II. with change(s) in first and second chemotherapy gene expression data sets from corresponding tumor types known to be poor responders to the same chemotherapy is predictive of poor response of the tumor to the chemotherapy.

In one embodiment the step of analysing the change in gene expression to predict response of the tumor to the chemotherapy comprises comparing change(s) in gene expression from II. with change(s) in first and second chemotherapy gene expression data sets from corresponding tumor types known to be good responders to the same chemotherapy. Preferably, positive relationship of change(s) in gene expression from II. with change(s) in first and second chemotherapy gene expression data sets from corresponding tumor types known to be good responders to the same chemotherapy is predictive of good response of the tumor to the chemotherapy.

In some embodiments the method is repeated by using further chemotherapy gene expression data sets each obtained by performing gene expression analysis on a sample obtained from the patient after further exposure of the tumor to the chemotherapy in place of, or in addition to, either or both of the first and second chemotherapy gene expression data sets.

In some embodiments of the methods of the present invention one or more (or all) of the sample(s) on which gene expression analysis is performed is taken from tumor tissue in the patient.

Importantly, the inventors have discovered that samples taken remotely from the tumor, e.g. from the patient's blood, may also be used to successfully analyse gene expression signatures and predict tumor response. Accordingly, in other embodiments of the methods of the present invention one or more (or all) of the sample(s) on which gene expression analysis is performed is taken from a bodily fluid of the patient, e.g. blood.

### DESCRIPTION OF PREFERRED EMBODIMENTS

The methods of the present invention concern prediction of response of a tumor to chemotherapy and the selection of a further treatment for a tumor that has been exposed to chemotherapy.

The tumor may be of any kind. The tumor is preferably the result of a cancerous condition, i.e. any unwanted cell proliferation (or any disease manifesting itself by unwanted cell proliferation), or neoplasm or increased risk of or predisposition to the unwanted cell proliferation, or neoplasm. The tumor may be a cancer and may be a benign or malignant cancer and may be primary or secondary (metastatic). A tumor may be any abnormal growth or proliferation of cells and may be located in any tissue. Examples of tissues include the colon, pancreas, lung, breast, uterus, stomach, kidney, testis, central nervous system (including the brain), peripheral nervous system, skin, blood or lymph. Tumors may be nervous or non-nervous system tumours. In one embodiment the tumor is a breast tumor. The tumor is preferably a solid tumor.

Non-nervous system tumors may be of, or may originate from, any non-nervous tissue. Examples include melanoma, mesothelioma, lymphoma, hepatoma, epidermoid carcinoma, prostate carcinoma, breast tumors, lung tumors or colon tumors. Tumors may be carcinomas, e.g. squamous cell carcinomas. They may be of, or originate from, the lung, head, neck, oesophagus or cervix. Breast cancers within the scope of the invention may be selected from the group consisting of:
(i) invasive ductal carcinoma (IDC);
(ii) ductal carcinoma in situ (DCIS);
(iii) invasive lobular carcinoma (ILC);
(iv) invasive lobular carcinoma in situ (ILCIS);
(v) adenocarcinoma (AC);
(vi) lobular hyperplasia benign (LBH);
(vii) medullary carcinoma (MD).

Nervous system tumours may originate either in the central or peripheral nervous system, e.g. glioma, medulloblastoma, meningioma, neurofibroma, ependymoma, Schwannoma, neurofibrosarcoma, astrocytoma and oligodendroglioma.

The chemotherapy may also be of any kind. Chemotherapy refers to treatment of a tumor with a drug or with ionising radiation (e.g. radiotherapy using X-rays or γ-rays). In preferred embodiments chemotherapy refers to treatment with a drug. The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the tumor.

The chemotherapy may be administered by one or more routes of administration, e.g. parenteral, intravenous injection, oral, or intratumoural. Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Methods of the present invention include methods for selecting a treatment for further treatment of a tumor that has been exposed to chemotherapy. The treatment being selected is preferably a chemotherapy treatment. It may comprise one or a combination of: maintenance of the existing chemotherapy or treatment regime; discontinuing the existing chemotherapy or treatment regime; starting a new chemotherapy or treatment regime; maintenance of the existing chemotherapy but altering the existing treatment regime.

In some embodiments the further treatment involves continuing with the existing chemotherapy and supplementing that chemotherapy with a further chemotherapy, e.g. with administration of a new drug.

In some embodiments the further treatment involves modifying the existing chemotherapy, e.g. by changing the drug being administered but maintaining the treatment regime or modifying the existing treatment regime but maintaining the drug being administered.

In some embodiments the further treatment involves discontinuing the existing chemotherapy and replacing it with a new chemotherapy, e.g. with administration of a new drug.

Chemotherapeutic drugs may be selected from:
(i) alkylating agents such as cisplatin, carboplatin, mechlorethamine, cyclophosphamide, chlorambucil, ifosfamide;
(ii) purine or pyrimidine anti-metabolites such as azathiopurine or mercaptopurine;
(iii) alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel;
(iv) topoisomerase inhibitors such as the type I topoisomerase inhibitors camptothecins irinotecan and topotecan, or the type II topoisomerase inhibitors amsacrine, etoposide, etoposide phosphate, teniposide;
(v) antitumor antibiotics (e.g. anthracyline antibiotics) such as dactinomycin, doxorubicin (Adriamycin™), epirubicin, bleomycin, rapamycin;
(vi) antibody based agents, such as anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab.

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D, Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte-Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, lxabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

Some embodiments of the present invention concern treatment with doxorubicin (Adriamycin™) and/or docetaxel.

Doxorubicin is an anthracyline antibiotic, related to the natural product daunomycin. It intercalates DNA. Doxorubicin is typically intravenously administered in the form of the hydrochloride salt.

Doxetaxel (Taxotere™) is an taxane and an anti-mitotic, commonly used in conjunction with anthracyline-based chemotherapy, e.g. with doxorubicin. A typical treatment regime comprises administration as a one-hour infusion every three weeks, over a ten-course cycle.

Methods of the present invention involve performing gene expression analysis on a sample obtained from a patient having a tumor that has (or has not) been exposed to chemotherapy so as to obtain a chemotherapy or baseline gene expression data set.

In some embodiments the sample is taken from tumor tissue in the patient.

In other embodiments the sample is taken from a bodily fluid, preferably one that circulates through the body. Accordingly, the sample may be a blood sample or lymph sample. In particular, the sample may comprise or may be derived from: a quantity of blood; a quantity of serum derived from the individual's blood which may comprise the fluid portion of the blood obtained after removal of the fibrin clot and blood cells; or cells isolated from said bodily fluid. A blood derived sample may be a selected fraction of a patient's blood, e.g. a selected cell-containing fraction or a plasma or serum fraction. A selected cell-containing fraction may contain cell types of interest which may include white blood cells (WBC), particularly peripheral blood mononuclear cells (PBMC) and/or granulocytes, and/or red blood cells (RBC).

In preferred embodiments, obtaining the sample does not form part of the invention. The sample may be obtained by tissue biopsy or during surgical resection, or by taking a blood sample. The sample may comprise or may be derived from a tissue sample, biopsy or isolated cells from said individual. The sample may be stored, e.g. frozen, prior to gene expression analysis. Samples may be processed in order to extract a particular component, e.g. DNA, RNA, protein, for gene expression analysis.

Samples may be obtained for gene expression analysis at predetermined time intervals during chemotherapy. For example, in embodiments comprising a treatment regime involving rounds (cycles) of chemotherapy, samples may be taken after one or each round of chemotherapy. In other embodiments, samples may be taken at one or more predetermined time points during chemotherapy. For example, a sample may be taken 24, 48, 72, 96, 120, 144 or 168 hours after the start of a chemotherapy (or round of chemotherapy). Samples may be taken at regular time intervals, e.g. after 1, 2, 3, 4, 5, 6, or 7 days, or after 1, 2, 3, 4, 5, 6, 7, 8, 9, 10, 11 or 12 weeks, or after 3, 4, 5, 6, 7, 8, 9, 10, 11 or 12 months during the chemotherapy. Blood samples and blood derived samples are well-suited for regular, even daily, sampling.

The gene expression analysis is preferably performed in vitro. In some embodiments gene expression analysis comprises determining the mRNA expressed by cells contained in the sample. This can be achieved using commercially available microarray technology (e.g. from Affymetrix, Qiagen, or Roche), in which hybridization between complementary mRNAs in the sample and DNA adhered to a microarray chip are used to generate signals indicative of gene expression in the sampled tissue/fluid/cells.

DNA microarrays can be used to measure changes in gene expression levels, to detect single nucleotide polymorphisms (SNPs), in genotyping or in resequencing mutant genomes. They typically consist of an arrayed series of thousands of microscopic spots of DNA oligonucleotides, each containing picomoles of a specific DNA sequence which may be a short section of a gene or other DNA element that are used as probes to hybridize a cDNA or mRNA sample under high-stringency conditions. Probe-target hybridization may be detected and quantified by detection of fluorophore-, silver-, or chemiluminescence-labeled targets to determine relative abundance of nucleic acid sequences in the target.

The probes are typically attached to a solid surface, by a covalent bond to a chemical matrix (e.g. via epoxy-silane, amino-silane, lysine, or polyacrylamide). The solid surface can be glass or a silicon chip.

In accordance with some embodiments of the present invention the results of gene expression analysis performed on a sample obtained from a patient having a tumor are compared with those obtained from a patient having a corresponding tumor type.

A corresponding tumor type is preferably a tumor from the same tissue type, more preferably occurring in the same type of patient (i.e. human or animal), even more preferably in the same sex patient. For example, for treatment of female human breast cancer, a corresponding tumor type would be a breast cancer from another female human patient.

Whilst not essential to the present invention it is preferable to match the corresponding tumor type as closely as possible to the tumor being treated. This may include matching in terms of histological classification of the tumor and/or expression of biochemical markers, e.g. ER+/ER− and/or EGFR+/EGFR− for human breast cancer.

In accordance with some embodiments of the present invention the corresponding tumor type has been exposed to a corresponding chemotherapy. A corresponding chemotherapy is preferably one of the same chemical or radiation class. For example, both chemotherapy treatments may comprise treatment with an alklylating agent or with chemotherapeutic drugs from one of groups (i) to (vi) recited above. A corresponding chemotherapy may refer to treatment with the same combination of chemotherapeutics or to treatment with one of two or more chemotherapeutics used in co-therapy.

More preferably, the chemotherapy used to treat the corresponding tumor comprises the same active agent(s), for example a chemical entity or a biological agent that has the same or approximately the same structure and/or function to the chemotherapeutic used to treat the tumor of ongoing concern. Pharmaceutical salts, esters, solvates of a chemotherapeutic drug are considered to be within the definition of corresponding chemotherapy.

Although not essential to the methods of the present invention, in some embodiments the corresponding chemotherapy will have been applied to the corresponding tumor in accordance with a corresponding treatment regime. A corresponding treatment regime is an identical or equivalent treatment regime which may be intended to give an equivalent dosage of chemotherapy over a round of chemotherapy or over the entire course of chemotherapy taking account of differences in the patients being treated, e.g. differences in height, weight, sex, severity and stage of disease.

In accordance with some embodiments of the present invention the corresponding tumor is one for which gene expression data has been related with the response of that tumor type to the chemotherapy used. Accordingly, the corresponding tumor type is preferably a tumor for which gene expression analysis has been obtained during the course of chemotherapy in other patients. This may involve collection of gene expression data for a plurality of patients (e.g. as part of a clinical trial) having a particular tumor type who are undergoing the same or a corresponding chemotherapy. Gene expression data from the patients may be collected prior to the chemotherapy (to provide baseline tumor gene expression data) and one or more times during the chemotherapy (to provide chemotherapy gene expression data from one or more stages or time points during the chemotherapy). Gene expression data from each patient may be related with the outcome of the chemotherapy, which may be classified in terms of poor or good response to treatment and/or progression-free survival (PFS) or non-progression free survival (non-PFS). Relating the gene expression data with tumor response preferably involves establishing a relationship, e.g. a concordance or correlation (e.g. statistical relationship, statistical concordance, statistical closeness of fit or statistical correlation such as Pearson correlation) between the gene expression data and the tumor response.

Gene expression data may be collected and obtained for the corresponding tumor type in accordance with the generation of gene expression data for the tumor undergoing treatment. For example, samples may be taken from the tumor or from patient bodily fluids, as described above, and gene expression analysis performed.

The data collected may be analysed and statistically refined (e.g. by selecting average or median data points from individual patient data or from the patient group data) to provide baseline and chemotherapy gene expression data sets for the corresponding tumor type which can be related with the outcome of the chemotherapy.

By comparing the gene expression data collected during treatment of the tumor of ongoing concern with gene expression data from the corresponding tumor (preferably with such data that has been collected at the same or an equivalent stage of the chemotherapy) one can statistically determine similarities in the data. Such analysis enables determination of the presence or absence of a positive relationship with patients having a corresponding tumor type that were found to be good or poor responders, or exhibit PFS or non-PFS. This comparison thereby enables one to predict the response of the tumor presently being treated to the ongoing chemotherapy. Particularly, in the case of prediction of poor response or non-PFS this provides an opportunity prior to conclusion of the existing chemotherapy to intervene by discontinuing, modifying, or supplementing the existing chemotherapy and/or by commencing a new treatment, which may be a new chemotherapy.

Gene expression analysis may indicate an increase in expression (up-regulation) or decrease in expression (down-regulation) of certain genes in the sampled tissue/fluid/cells (e.g. see Tables 3 and 4). This up-regulation or down regulation may be compared against the up-regulation and down regulation of the same or corresponding genes in another gene expression data set, e.g. one from a corresponding tumor.

Comparison of up-regulation or down-regulation of one or more corresponding genes between gene expression data sets may therefore be used to predict tumor response for a patient undergoing treatment. Comparisons may be made of gene expression signatures obtained from tumor prior to treatment (T1), after or during treatment (T2), and/or the relative changes between T1 and T2 (TΔ) with respect to their ability to predict tumor response.

In some embodiments a comparison may involve:
1. Identifying genes that are differentially expressed in good and/or poor responders to the chemotherapy. This may be determined for a corresponding tumor to generate gene expression data useful for comparing with gene expression data in patients undergoing chemotherapy; and/or
2. Performing statistical comparison between gene expression data. For example, performing statistical comparison between chemotherapy gene expression data from a patient undergoing treatment with gene expression data from a corresponding tumor.
3. Determining a relationship between the data and using said relationship to predict tumor response. For example, this may involve determining a correlation (e.g. Pearson correlation), concordance or other statistical measure of similarity or closeness of fit of response between the chemotherapy gene expression data from the patient undergoing treatment with gene expression data from the corresponding tumor. A positive relationship may be used as a predictor of a similar tumor response in the patient undergoing treatment to that reported for the corresponding tumor.

Comparisons may take the form of statistical analysis. Statistical analysis may involve use of independent t-tests to select genes that are differentially expressed by good and poor responders. Bootstrapping techniques may be used to provide a short-list of differentially expressed genes suitable for use in making comparisons between gene expression data for the purpose of predicting tumor response. This short-list may form a gene expression data set.

For statistical comparisons between gene expression data, Kaplan-Meier estimation may be used²¹. Kaplan-Meier curves are particularly well-suited to medical statistics because they can take account of losses from a sample before the final outcome is observed (e.g. withdrawal from a clinical trial or death of a patient). They are also suitable for use where patients have been grouped into particular categories, e.g. those with a particular gene expression profile.

The log rank test (Mantel-Cox test) may be applied to Kaplan-Meier curves to compare tumor response predictions. Univariate analysis may be performed to identify clinical and genetic predictors of tumor response. Multivariate analysis with Cox regression may also be used.

Statistical analyses may be performed using commercially available software packages such as the SPSS software version 13 (SPSS Inc., Chicago, USA).

Tumor response may be generally classified as good or poor. A poor response may indicate failure to limit the spread of disease (e.g. increase, or no reduction, in tumor size), two or more axillary lymph nodes involved with tumor, and/or disease progression (e.g. metastasis). A good response may indicate a reduction in tumor size and/or reduction in number of tumors and/or no further metastases.

Tumor response may be further sub-classified in terms of “pathological response” and “survival”. Poor and good pathological responders and short and long-term survivors may be identified. For a corresponding tumor this may be done in the context of a group of patients having a corresponding tumor who received a corresponding chemotherapy. Poor and good pathological responders may be derived by dividing the group at approximately the median based on pathological tumor size, nodal status and/or progression-free survival. Short and long-term survivors may be similarly derived by dividing the group at approximately the median based on survival following treatment. Tumor response may also be classified based on resistance to the chemotherapy. “Resistant” and “Sensitive” tumors may be identified.

In some embodiments tumor response may be classified as good where patients are predicted to survive for one of at least 1 year, 18 months, 2 years, 3 years, 4 years or 5 years from commencement of the chemotherapy or from completion of the chemotherapy. In some embodiments tumor response may be classified as poor where patients are predicted to survive for one of less than 2 years, 18 months, 1 year, or 6 months from commencement of the chemotherapy or from completion of the chemotherapy.

Patients exhibiting progression-free survival for at least one of 1 year, 18 months, 2 years, 3 years, 4 years or 5 years from commencement of the chemotherapy or from completion of the chemotherapy may be considered good responders and/or long-term survivors.

Patients exhibiting non-progression-free survival for at least one of 3 years, 4 years or 5 years from commencement of the chemotherapy or from completion of the chemotherapy may be considered poor responders, but long-term survivors.

In some embodiments, methods according to the present invention may therefore comprise a step of providing a prediction of patient response to the tumor treatment. This may be a prediction of good or poor response and/or short or long-term survival. The prediction is comparative, based on comparison of gene expression between the patient undergoing treatment and data collected for patients previously treated. The prediction may be made with a specified statistical degree of confidence or confidence interval (e.g. 95% confidence interval (CI)).

Methods according to the present invention are preferably performed in vitro. The term “in vitro” is intended to encompass experiments with cells in culture whereas the term “in vivo” is intended to encompass experiments with intact multi-cellular organisms. Where the method is performed in vitro it may comprise a high throughput gene expression analysis assay.

Methods according to the present invention may optionally not comprise a step of administering a treatment, e.g. chemotherapy, to a patient. In preferred embodiments the selection of a treatment does not comprise the administration of that treatment. As such, methods of prediction or selection according to the present invention do not comprise a method of treatment of the human or animal body by surgery or therapy.

The patient/subject being treated may be any animal or human. The subject is preferably mammalian, and more preferably is human. The subject may be male or female.

In some embodiments methods according to the present invention concern patients having breast tumor receiving a chemotherapeutic treatment comprising administration of one or more of an antitumor antibiotic (e.g. an anthracyline antibiotic such as doxorubicin) and/or a taxane (e.g. docetaxel).

Gene expression data sets are generated for at least one of: tumor prior to treatment (T1), tumor after or during treatment (T2), and/or for the relative changes in gene expression between T1 and T2 (TΔ). Data sets are generated for pathological response and/or progression-free survival. Example data sets are shown in FIGS. 6 and 7 (Tables 3 and 4). Examples of refined data sets containing fewer genes are shown in FIGS. 8 and 9 (Tables 5 and 6). For convenience, the genes identified in FIGS. 6 and 7 are given a number between 0-199.

Thus in some embodiments prediction of response of breast tumor to antitumor antibiotic (e.g. doxorubicin) and/or taxane (e.g. docetaxel) treatment, and/or selection of further tumor treatment, involves selecting one or more of genes 0-199 from one of FIG. 6, 7, 8 or 9 (Tables 3, 4, 5, or 6) and comparing expression of that gene with expression of the corresponding gene (preferably the same gene) from a corresponding chemotherapy gene expression data set generated for the tumor undergoing treatment. For example, where a gene from a T1 data set is selected, the comparison is made with a corresponding gene from a T1 data set generated for the tumor undergoing treatment. Where a gene from a T2 data set is selected, the comparison is made with a corresponding gene from a T2 data set generated for the tumor undergoing treatment. Where a gene from a TΔ data set is selected, the comparison is made with a corresponding gene from a TΔ data set generated for the tumor undergoing treatment.

All of the genes in FIG. 6, 7, 8 or 9 (Tables 3, 4, 5, or 6) may be selected but in many preferred embodiments a subset of those genes are selected to make the comparison. This subset may comprise one of 10% or more, 20% or more, 30% or more, 40% or more, 50% or more, 60% or more, 70% or more, 80% or more, 90% or more, and/or one of 90% or less, 80% or less, 70% or less, 60% or less, 50% or less, 40% or less, 30% or less, 20% or less, 10% or less of genes 0-199 in one of FIG. 6A, 6B, 6C, 7A, 7B, 7C or of the genes in one of FIG. 8A, 8B, 8C, 8D, 8E, 8F, or 9A, 9B, 9C, 9D, 9E, 9F.

In some embodiments, groups of genes may be selected to perform the comparison. For example, one may select 10 or more, 20 or more, 30 or more, 40 or more, 50 or more, 60 or more, 70 or more, 80 or more, 90 or more, or 100 or more genes from one of FIGS. 6A, 6B, 6C, 7A, 7B, 7C. In some embodiments, one may select less than 100, less than 90, less than 70, or less than 50 genes from one of FIGS. 8A, 8B, 8C, 8D, 8E, 8F, or 9A, 9B, 9C, 9D, 9E, 9F.

In some embodiments methods according to the present invention involve analysing the gene expression of one or more (or all) of the genes listed in FIGS. 16, 18 and 20 (Tables 11, 12 and 13). For example, in some embodiments gene expression analysis may involve comparing the expression of 1, 2, 3, 4, 5, 6 or 7 of the genes listed in FIG. 16 (Table 11). In some embodiments gene expression analysis may involve comparing the expression of 1, 2, 3, 4, 5, 6, 7, 8, 9, 10, 11, 12, or 13 of the genes listed in FIG. 18 (Table 12). In some embodiments gene expression analysis may involve comparing the expression of 1, 2, 3, 4, 5, 6, 7, 8, 9, or 10 of the genes listed in FIG. 20 (Table 13).

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein, by reference.

### BRIEF DESCRIPTION OF THE FIGURES

Embodiments and experiments illustrating the principles of the invention will now be discussed with reference to the accompanying figures in which:

FIG. 1.

Application of 90-supergene panel for progression-free survival into the original cohort of 47 patients. (a) Unsupervised hierarchical clustering of samples using the 90-supergene panel for T1, T2, and TΔ; each row represents a gene, and each column represents a patient sample; long survivors are indicated in blue, short survivors are indicated in orange; genes that are increased in expression are in red, while those that are reduced in expression are in green. (b) Receiver operating characteristic curves for T1, T2 and TΔ gene signatures in predicting for progression-free survival using the top 90 supergenes. (c) Kaplan-Meier plots for progression-free survival by groups (short or long survivors) predicted by T1, T2 and TΔ gene signatures. The median survival for the groups predicted to be short survivors by T1, T2 and TΔ gene signatures were 17.3 months, 12.0 months, and 13.0 months respectively, while the median survival for the groups predicted to be long survivors for all 3 gene panels was not reached.

FIG. 2.

Validation of the 90-supergene panels for progression-free survival and pathological response in 2 independent validation sets. (a) Receiver operating characteristic curves plotted using absolute SVM scores for T1, T2 and TΔ gene signatures in predicting for relapse-free survival in an independent adriamycin validation set (GDS84, n=20) using the top 90 supergenes. (b) Kaplan-Meier plots for progression-free survival by groups (short or long survivors) predicted by T1, T2, and TΔ gene signatures. The median survival for the groups predicted to be long and short survivors respectively were 33 months and not reached for T1 signatures, 39 months and 10 months for T2 signatures, and 59 months and 23 months for TΔ signatures. (c) Receiver operating characteristic curves plotted using absolute SVM scores for T1, T2 and TΔ gene signatures in predicting for pathological response in an independent docetaxel validation set (n=18) using the top 90 supergenes. (d) Scatterplot of samples in independent docetaxel validation set (n=18) in relation to SVM scores derived using T1, T2, and TΔ 90-supergene panels, for pathological response. Blue and pink dots represent samples that are ‘poor’ and ‘good’ pathological responders respectively. Samples are classified as ‘good pathological responder’ by the predictive panel if the SVM score is more than 0, and as ‘poor pathological responder’ if the SVM score is ≦0. The absolute SVM score corresponds to the probability of a test sample belonging to a certain group, with the probability increasing with increasing absolute value.

FIG. 3.

(a) Degree of similarity between lists of top 200 discriminatory genes for progression-free survival with each 100 incremental bootstrap, starting with 100 bootstraps. A total of 10,000 bootstraps were performed, but data is only shown up to 3200 bootstraps as plateau was reached for all 3 sets of predictive panels by 2200-3000 bootstraps. (b) Differences in mean AUC of ROC of predictive gene panels derived from training sets, comprising of different number of genes ranging from 50-200, in predicting PFS in corresponding test samples, based on 2000 resampled datasets, using AUC for the 50-gene panel as the reference.

FIG. 4. Table 1—Univariate and multivariate analysis of clinical and genetic factors that predict for progression-free survival in primary cohort (n=47).

FIG. 5. Table 2—Univariate and multivariate analysis of clinical and genetic factors that predict for relapse-free survival in independent adriamycin validation set (n=20, GDS84).

FIG. 6. Table 3—200 supergene panels for progression-free survival (PFS): (A) 200 supergene panel for T1 for PFS; (B) 200 supergene panel for T2 for PFS; (C) 200 supergene panel for TΔ for PFS. Accession numbers are given for the GenBank database (NCBI).

FIG. 7. Table 4—200 supergene panels for pathological response: (A) T1 pathological response supergenes; (B) T2 pathological response supergenes; (C) TΔ pathological response supergenes. Accession numbers are given for the GenBank database (NCBI).

FIG. 8. Table 5—Functional categories of supergenes. Progression-free survival supergene panels: (A) T1 90-supergene panel for PFS; (B) T2 90-supergene panel for PFS; (C) TΔ 90-supergene panel for PFS. Pathological response supergene panels: (D) T1 90 supergene panel for pathological response; (E) T2 90 supergene panel for pathological response; (F) TΔ 90 supergene panel for pathological response. Accession numbers are given for the GenBank database (NCBI).

FIG. 9. Table 6—Overlapping genes. Progression-free survival (PFS) 200-supergene panel: (A) Common genes between T1 and T2 for PFS; (B) Common genes between T1 and TΔ for PFS; (C) Common genes between T2 and TΔ for PFS; (D) Common genes between T1 and T2 for pathological response; (E) Common genes between T1 and TΔ for pathological response; (F) Common genes between T2 and TΔ for pathological response. Accession numbers are given for the GenBank database (NCBI).

FIG. 10. Table 7—Characteristics of primary cohort.

FIG. 11. Table 8—Characteristics of adriamyin validation cohort.

FIG. 12. Table 9—Characteristics of docetaxel validation cohort.

FIG. 13. Charts showing distribution and magnitude of gene expression changes induced by doxorubicin and docetaxel in tumor and PBMC.

FIG. 14. Diagram showing PBMC and tumor probe sets that are highly concordant in expression levels (Pearson correlation >0.9).

FIG. 15. Table 10—Functional categories of probe sets that are concordant between PBMC and tumor.

FIG. 16. Table 11—PBMC probe sets that were concordant with tumor probe sets to discriminate doxorubicin from docetaxel treatment,

FIG. 17. Gene expression data for Table 11. Red: Doxorubicin treated sample. Blue: Docetaxel treated sample.

FIG. 18. Table 12—PBMC probe sets that were concordant with tumor probe sets to discrimate sensitive from resistant tumors to doxorubicin.

FIG. 19. Gene expression data for Table 12. Red: Sensitive tumor to doxorubicin. Blue: Resistant tumor to doxorubicin.

FIG. 20. Table 13—PBMC probe sets that were concordant with tumor probes sets to discriminate sensitive from resistant tumors to docetaxel.

FIG. 21. Gene expression data for Table 13. Red: Sensitive tumor to docetaxel. Blue: Resistant tumor to docetaxel.

FIG. 22. Chart showing PBMC probe sets that discriminated between doxorubicin and docetaxel treatment. 18/21 (86%) samples correctly classified as docetaxel-treated.

FIG. 23. Chart showing PBMC probe sets that discriminated sensitive from resistant tumors to docetaxel. 10/12 (83%) sensitive tumors correctly classified. 6/9 (67%) resistant tumors correctly classified.

### DETAILED DESCRIPTION OF THE INVENTION

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

### Example 1

Using a set of 47 breast cancer patients, we developed gene panels of baseline tumor (T1), post-chemotherapy tumor (T2), and chemotherapy-induced relative change signatures (TΔ) to predict pathological response and progression-free survival. Validation of the signatures in 2 independent validation sets comprising of 18-20 patients each showed the post-chemotherapy signatures to be superior to baseline signatures in predicting these outcomes, suggesting that drug-induced tumor gene signatures may be more informative than unchallenged signatures. These findings have important clinical implications, and may be further exploited to uncover pathways as therapeutic targets in resistant tumors.

We developed baseline and chemotherapy-induced tumor gene expression signatures and compared their ability to predict treatment outcomes. The signatures were derived from 47 patients with measurable primary breast tumor who received six cycles of alternating sequential doxorubicin (A) and docetaxel (T) three-weekly, randomized to start with either drug in cycle one (A→T→A→T→A→T, n=25; or T→A→T→A→T→A, n=22), prior to definitive surgery. Tumor core biopsies were obtained before and approximately 3 weeks after the first chemotherapy cycle for gene expression analysis on the Affymetrix U133+2 microarray chip. Three sets of gene expression signatures, baseline tumor (T1), post-chemotherapy tumor (T2), and relative changes after chemotherapy ([T2-T1]/T1 or TΔ), were obtained for each subject. Treatment outcomes analyzed were pathological response and progression-free survival (PFS). To derive predictive gene panels, the cohort was divided at approximately the median based on pathological tumor size and nodal status and PFS, to yield two groups, poor and good pathological responders, and short and long survivors, respectively.

After stratifying for metastatic disease, the dataset was randomly divided into two-thirds and one-third as training and test sets respectively, and bootstrapping with 10,000 resampled datasets performed. With each bootstrap, the top discriminating genes for pathological response and PFS for T1, T2 and TΔ were derived from the training set, weighted using p-values, and tested in the corresponding test set. The top genes were continually refined with every 100 incremental bootstraps based on cumulative weights. A plateau in gene composition for the top 200 genes was reached by 2600-3000 bootstraps, with <1% dissimilarity between incremental bootstraps (FIG. 3a). A final list of the top 200 genes (termed supergenes) was created after 3000 bootstraps for T1, T2, and TΔ for pathological response and PFS (Tables 2-5). There was no overlap between the pathological response and PFS supergenes except for 2 genes for TΔ; 3-32 genes were common between the T1, T2 and TΔ panels for pathological response and PFS (Table 6). As predictive panels comprising of the top 50-200 genes in increments of 10 showed similar sample classification accuracy (FIG. 3b), we arbitrarily chose the 90-supergene panels for all subsequent analysis.

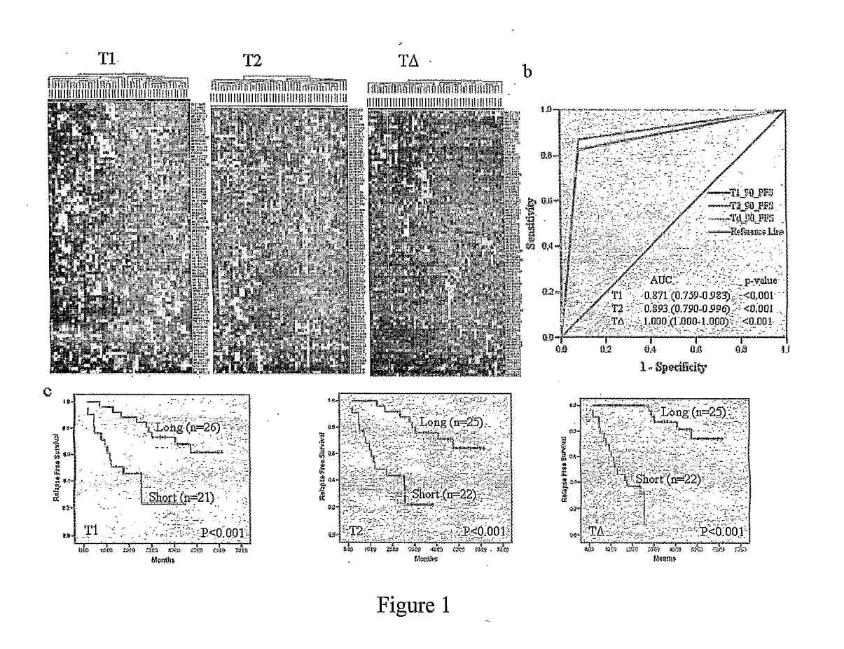

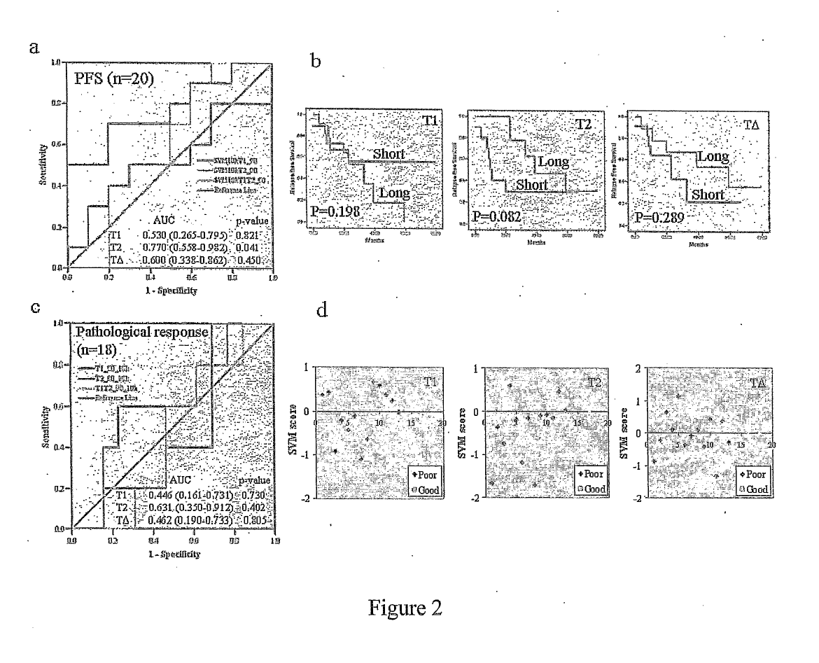

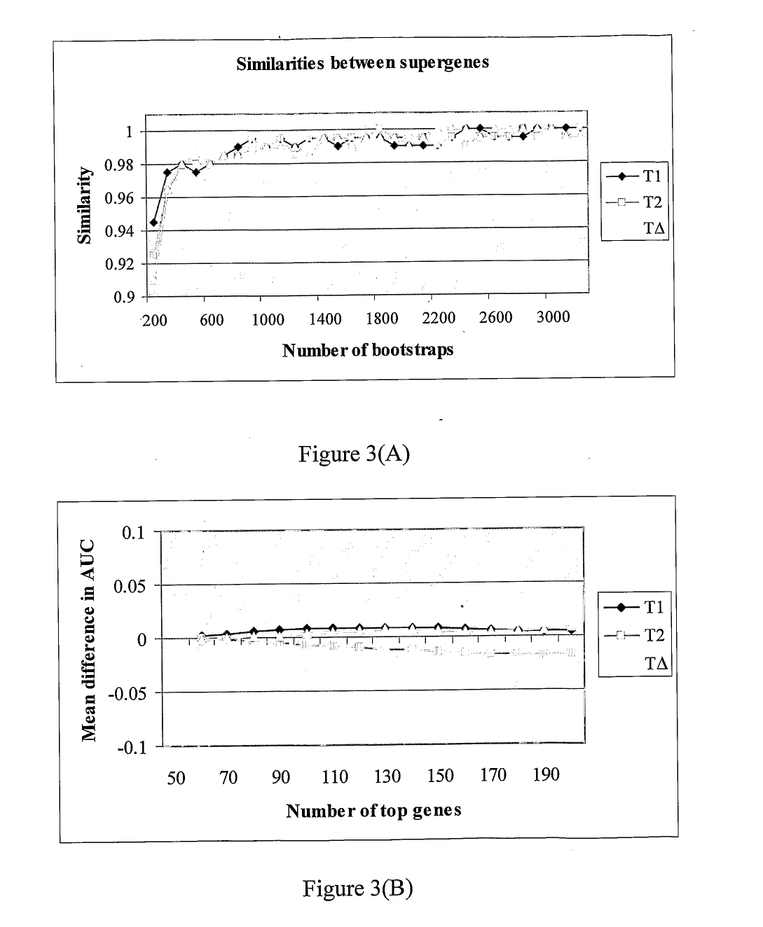

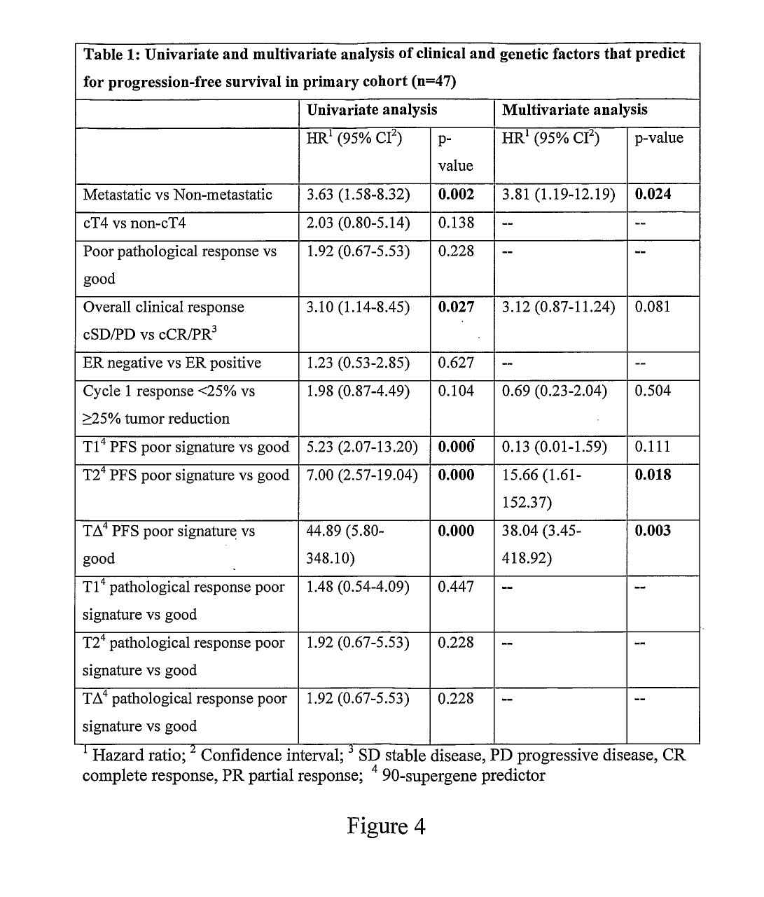

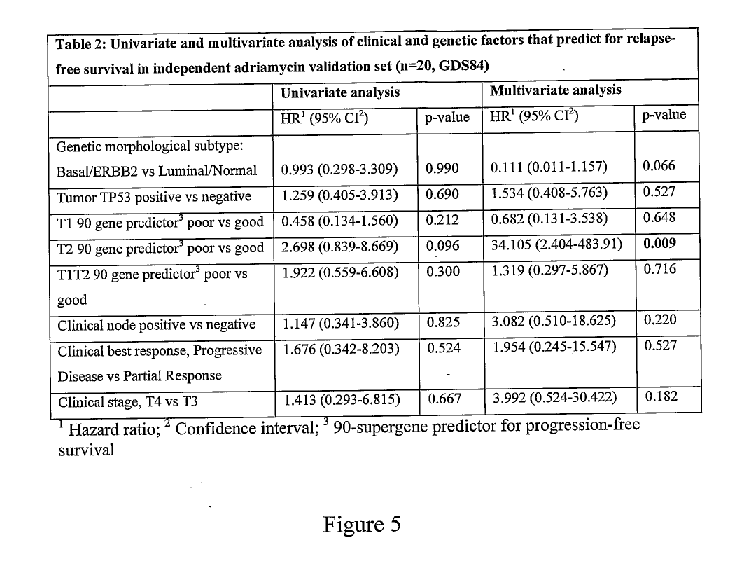

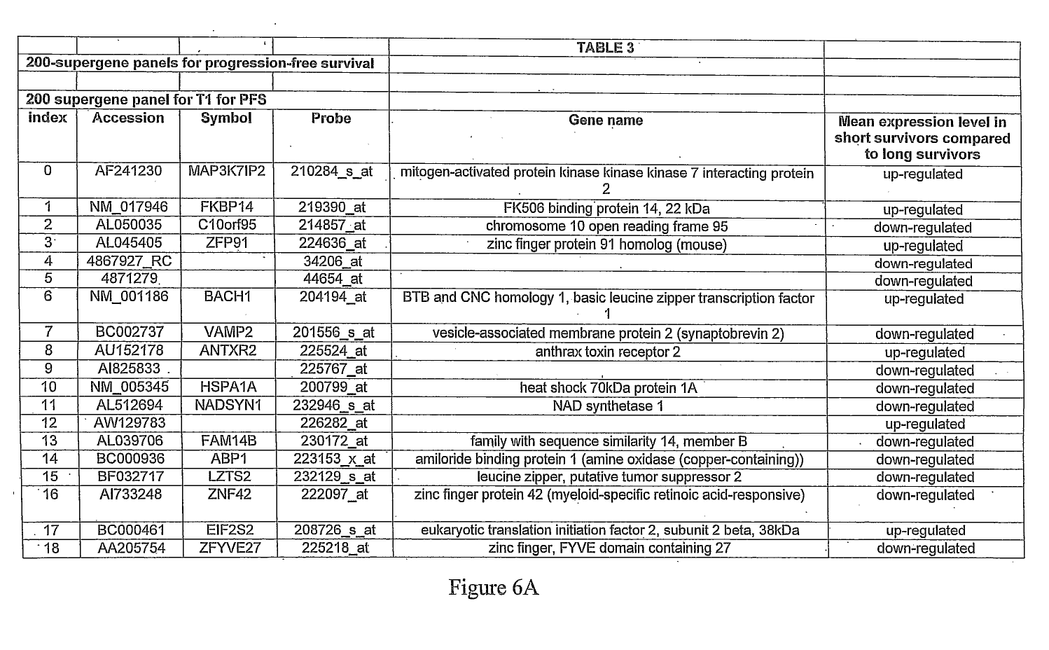

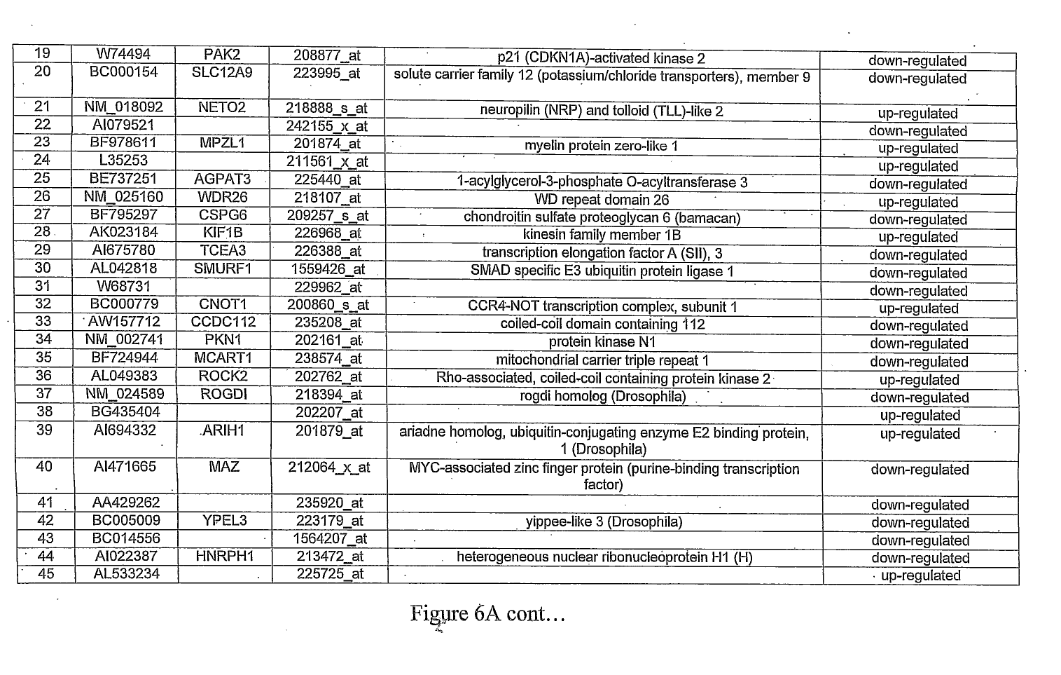

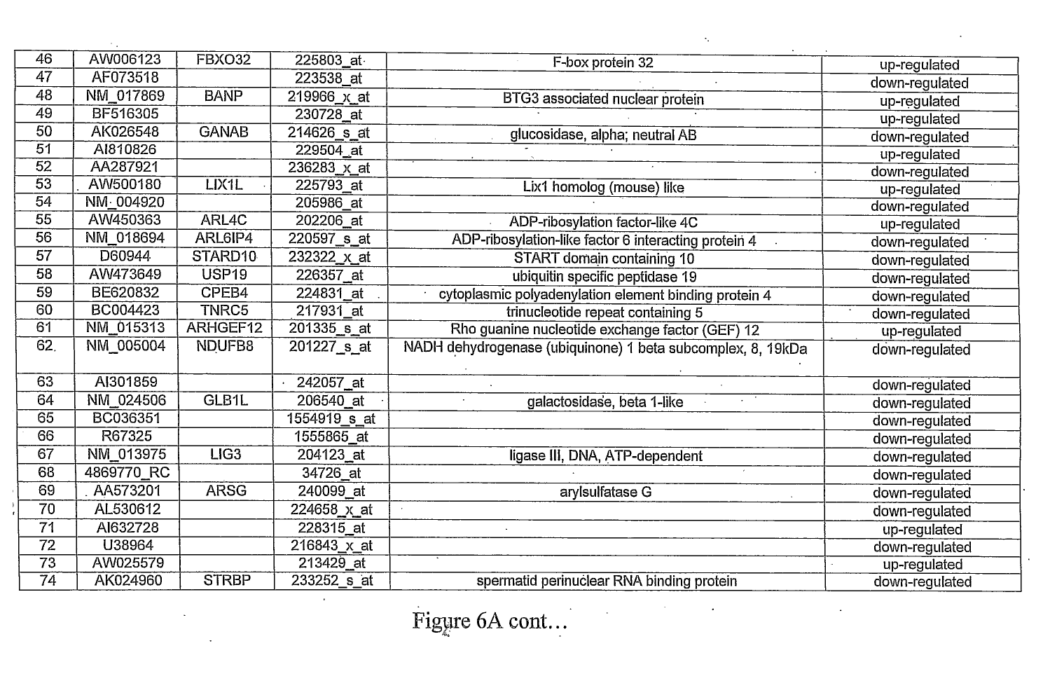

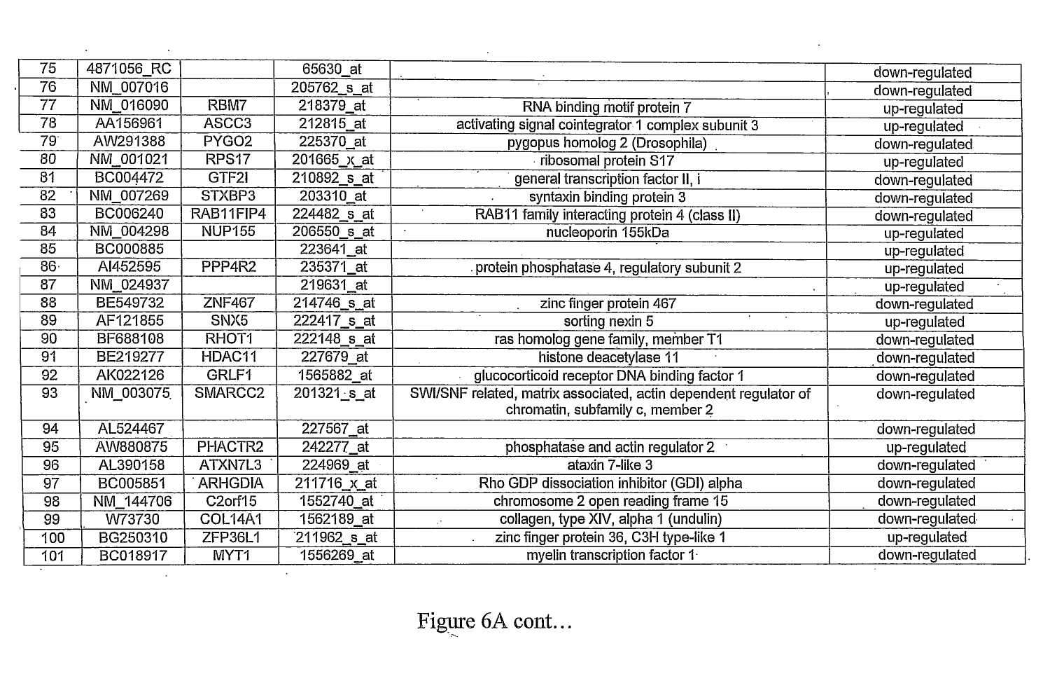

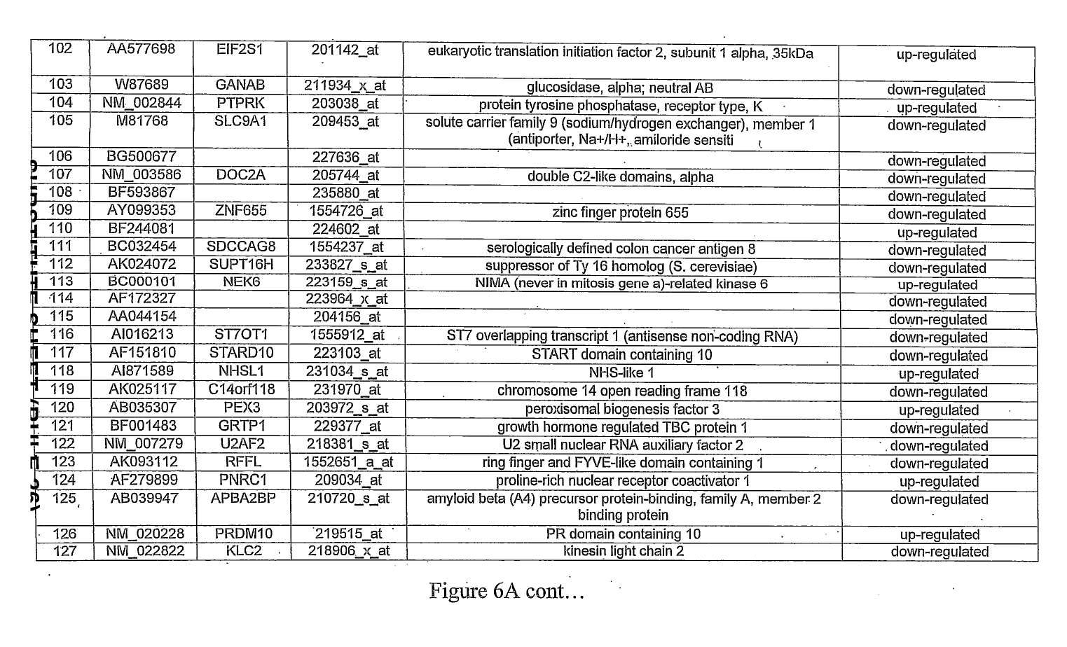

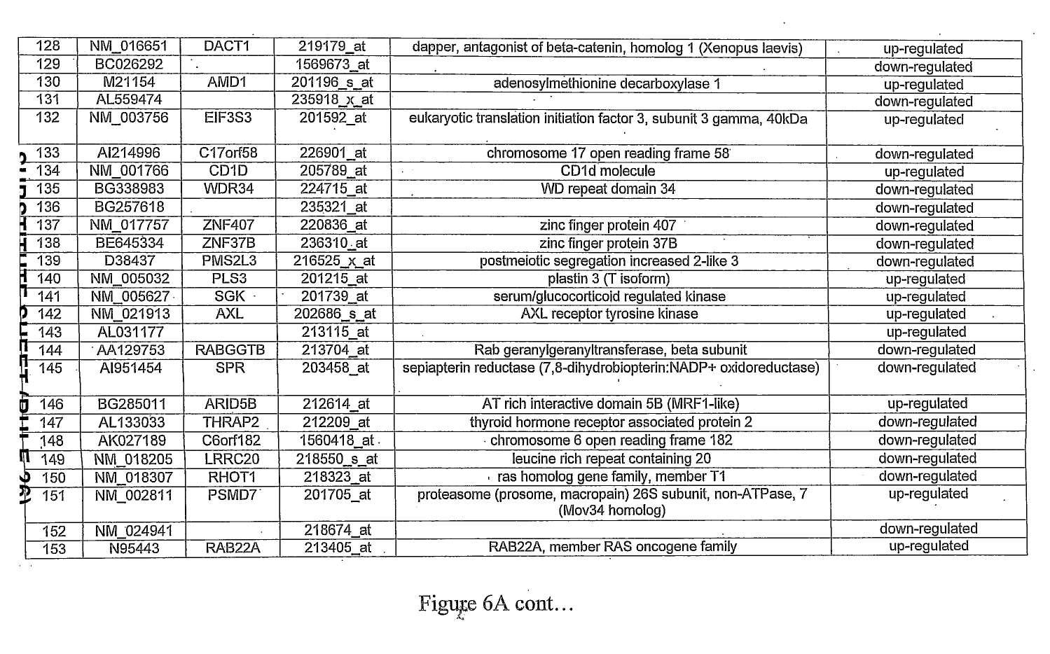

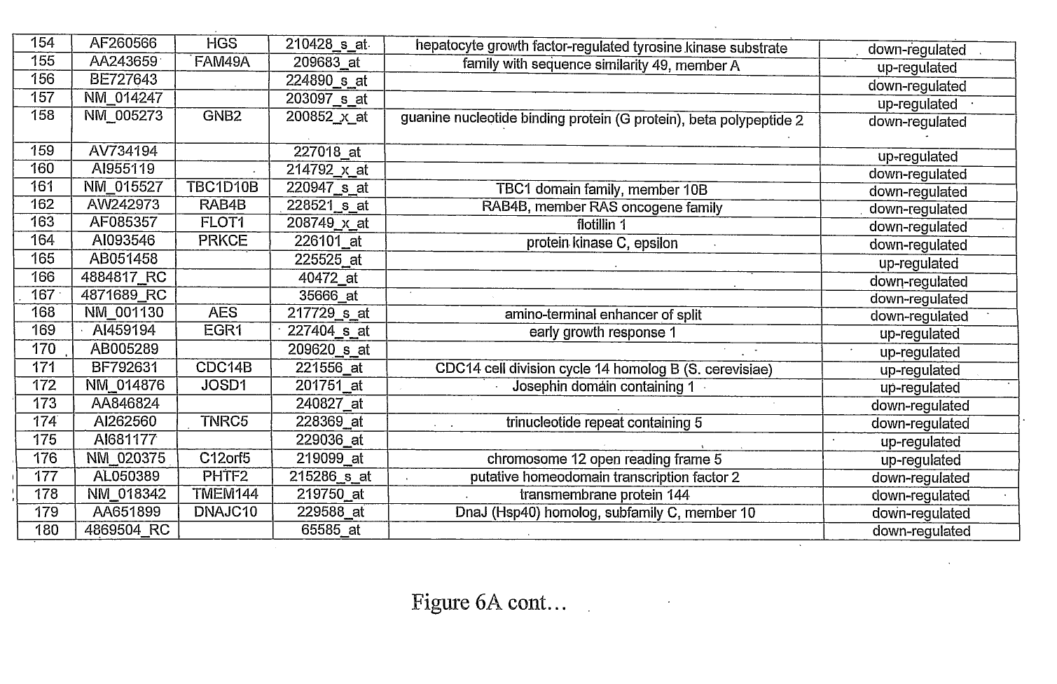

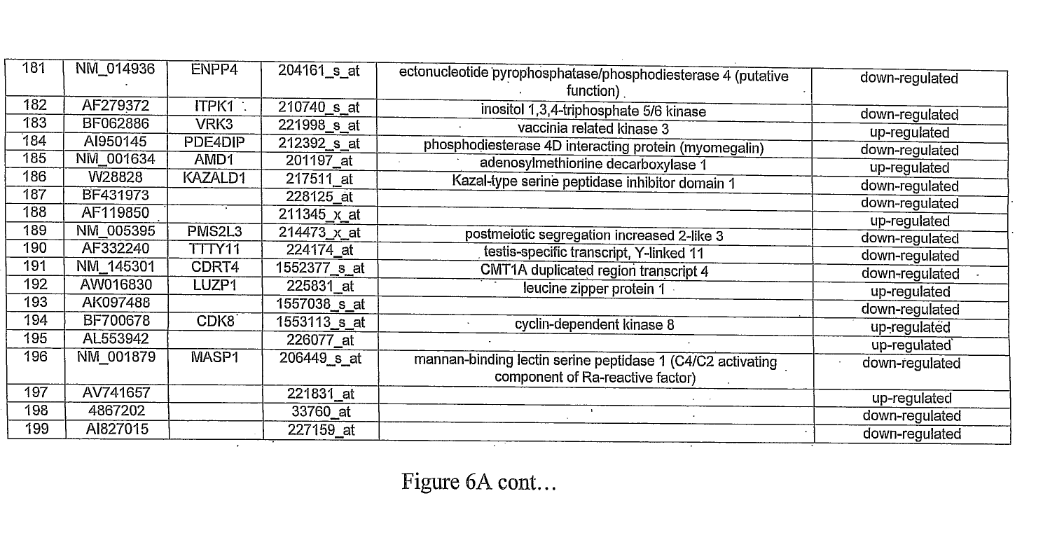

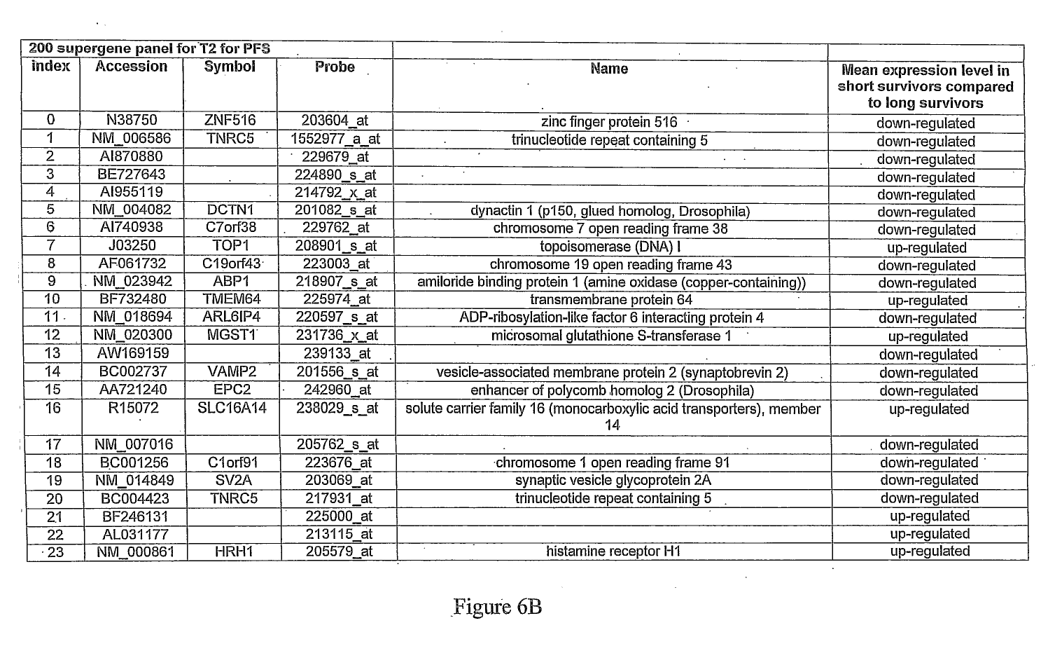

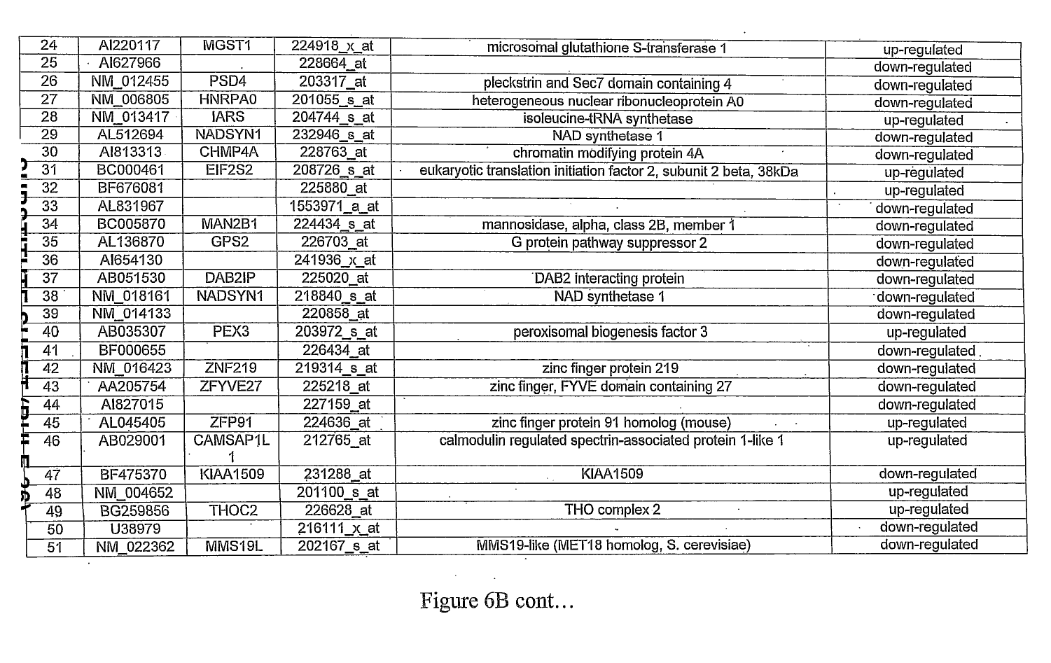

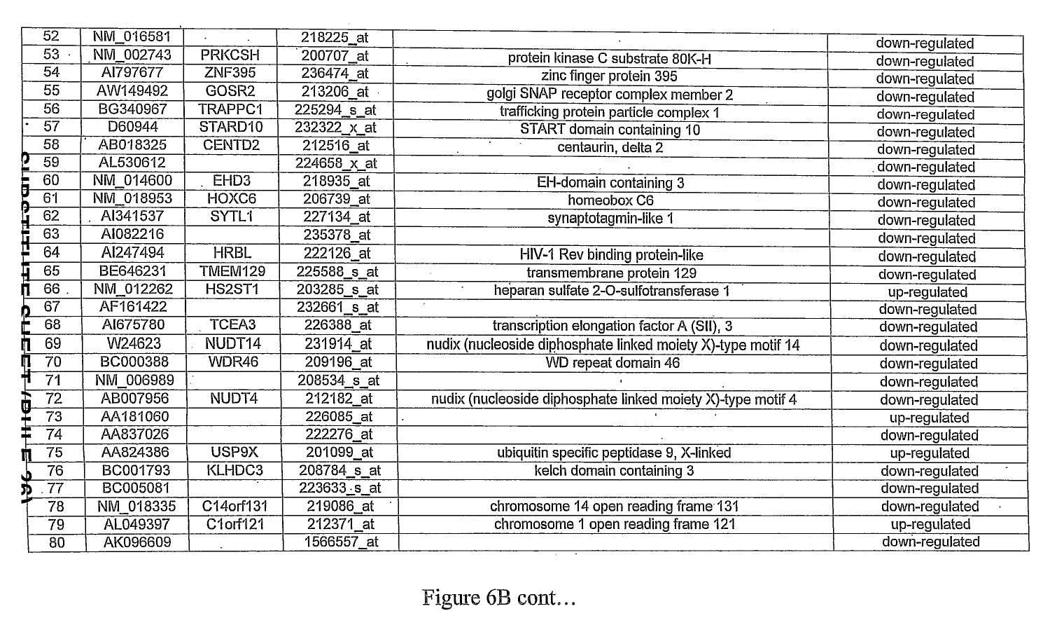

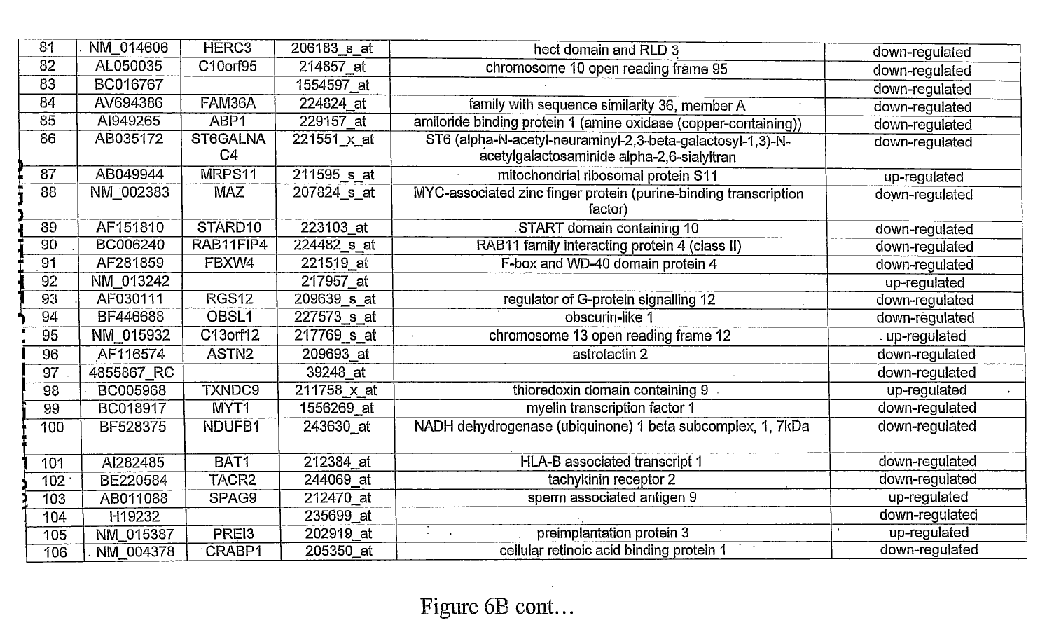

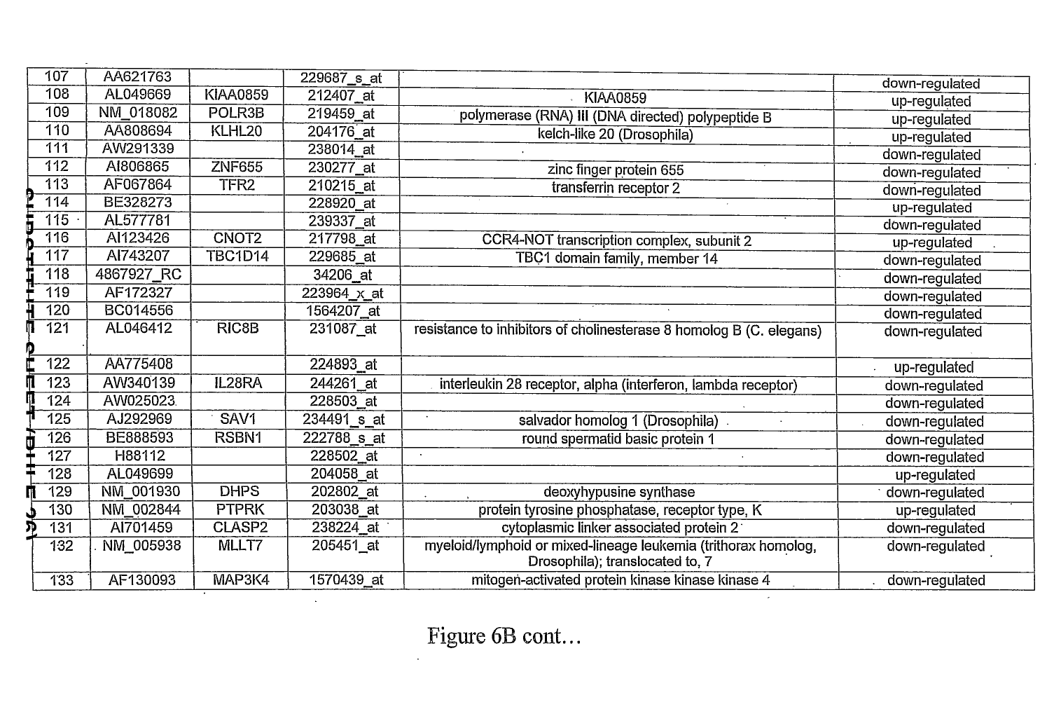

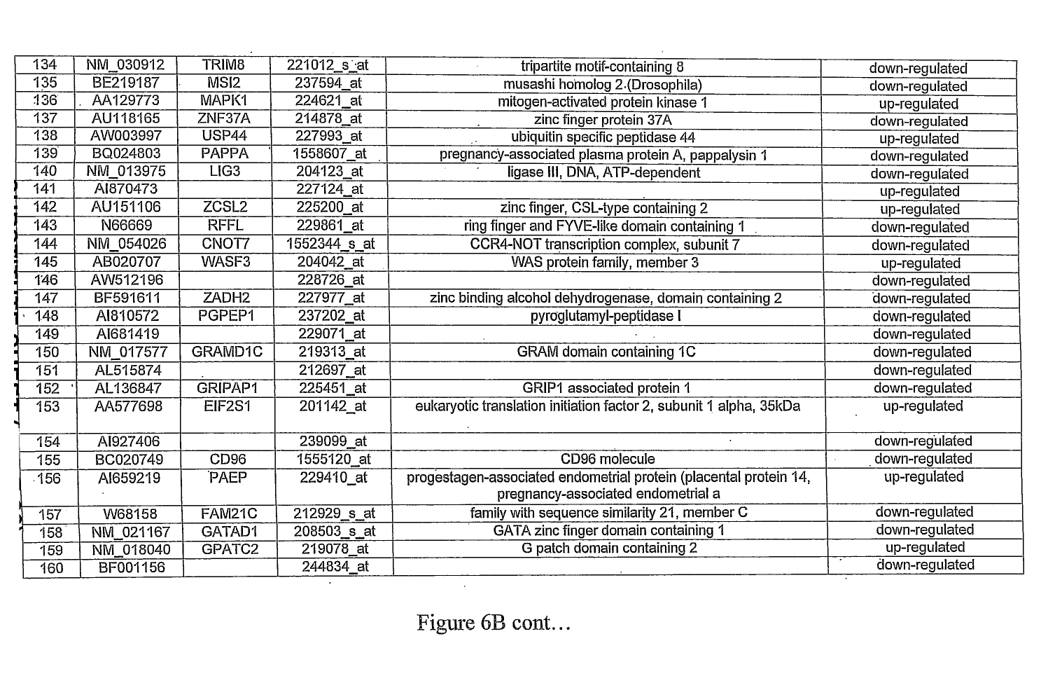

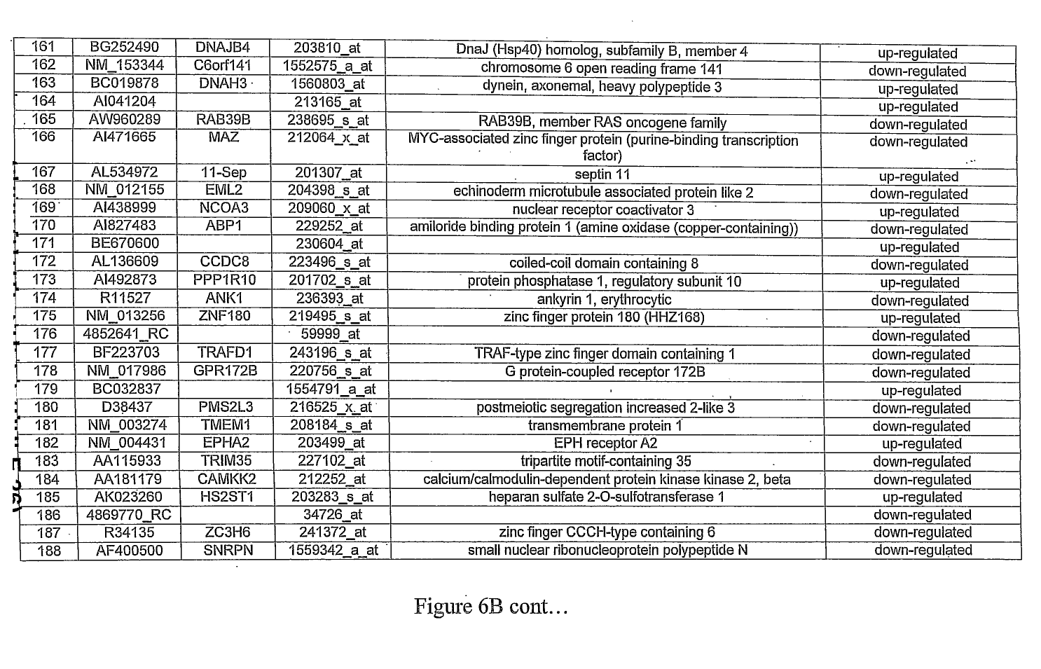

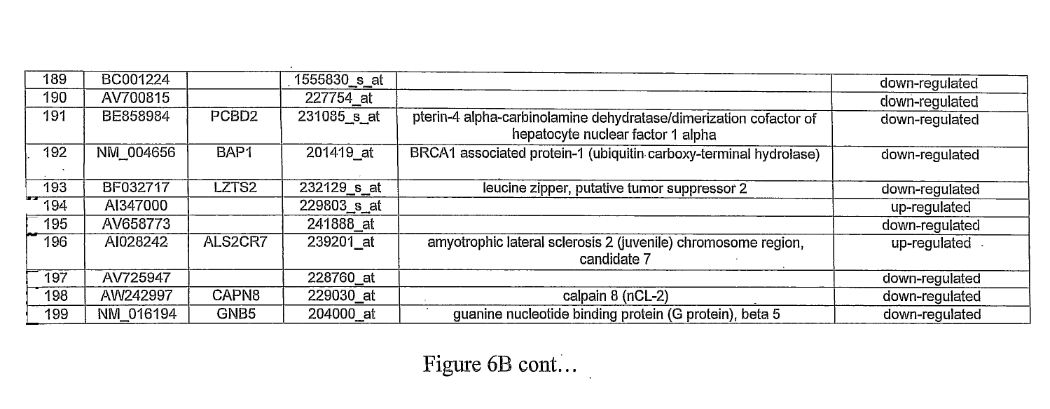

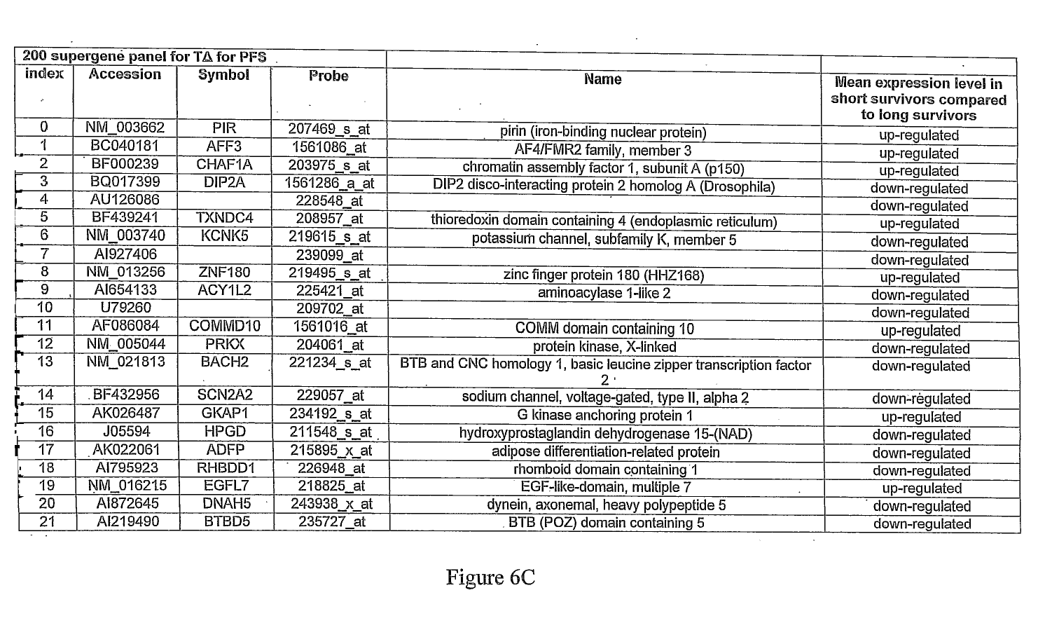

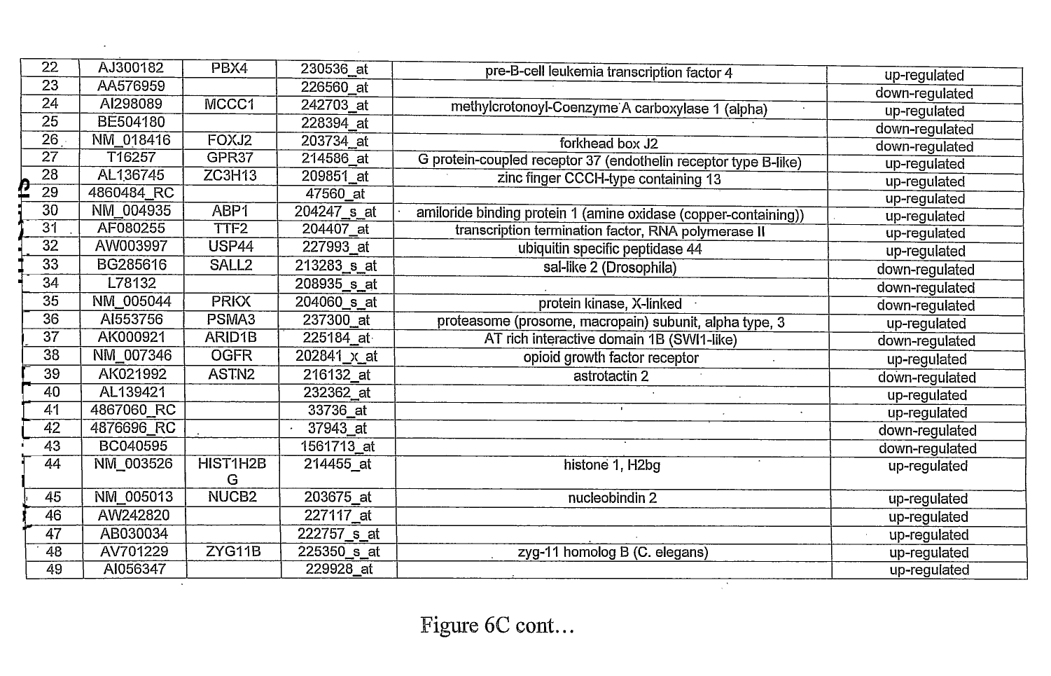

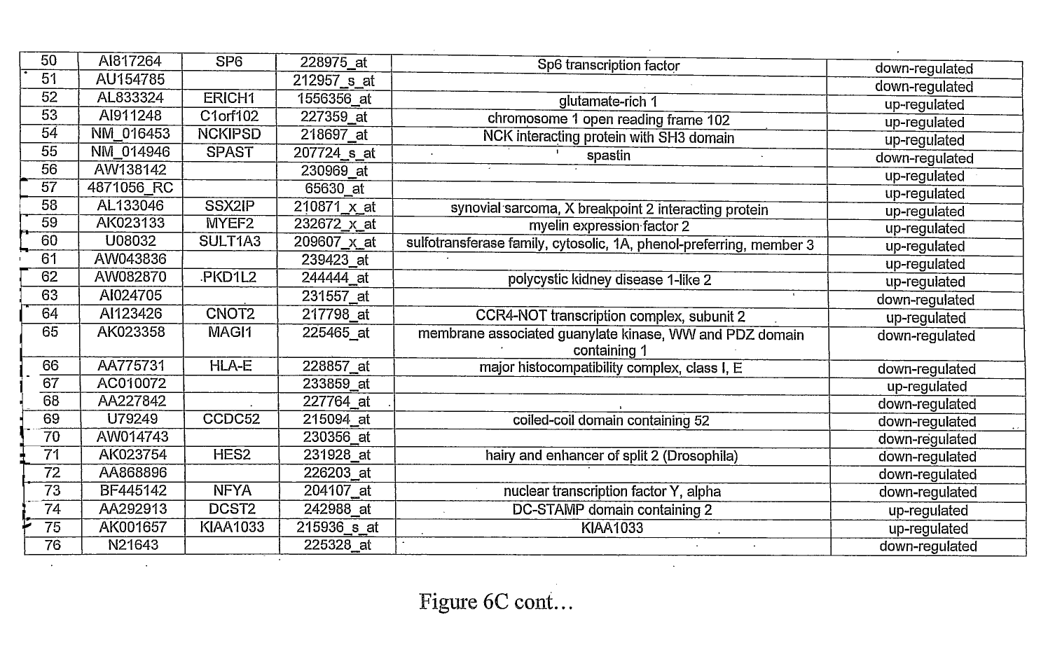

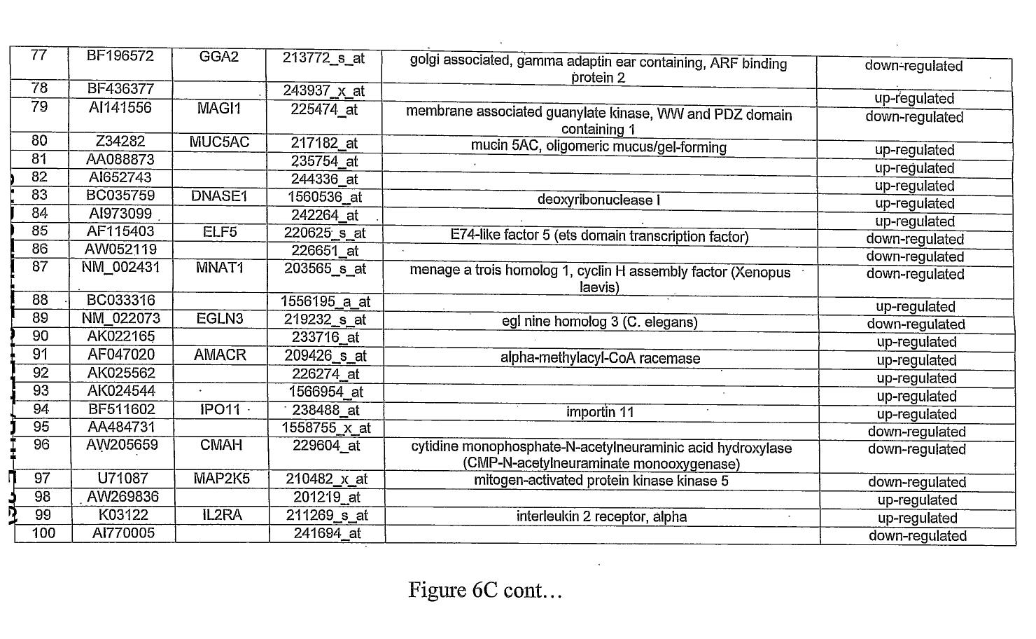

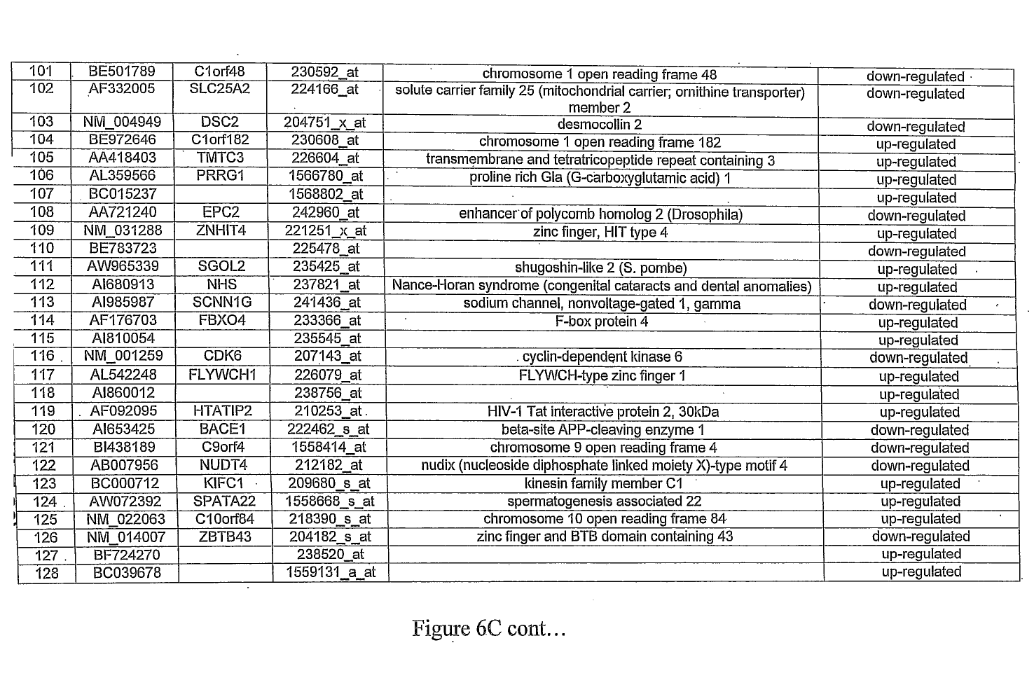

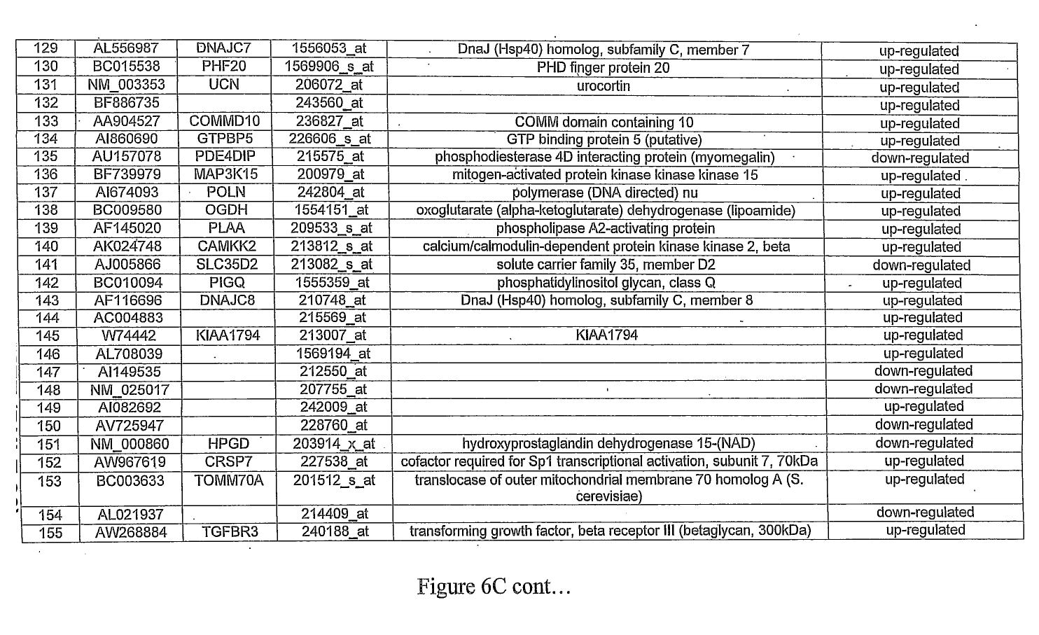

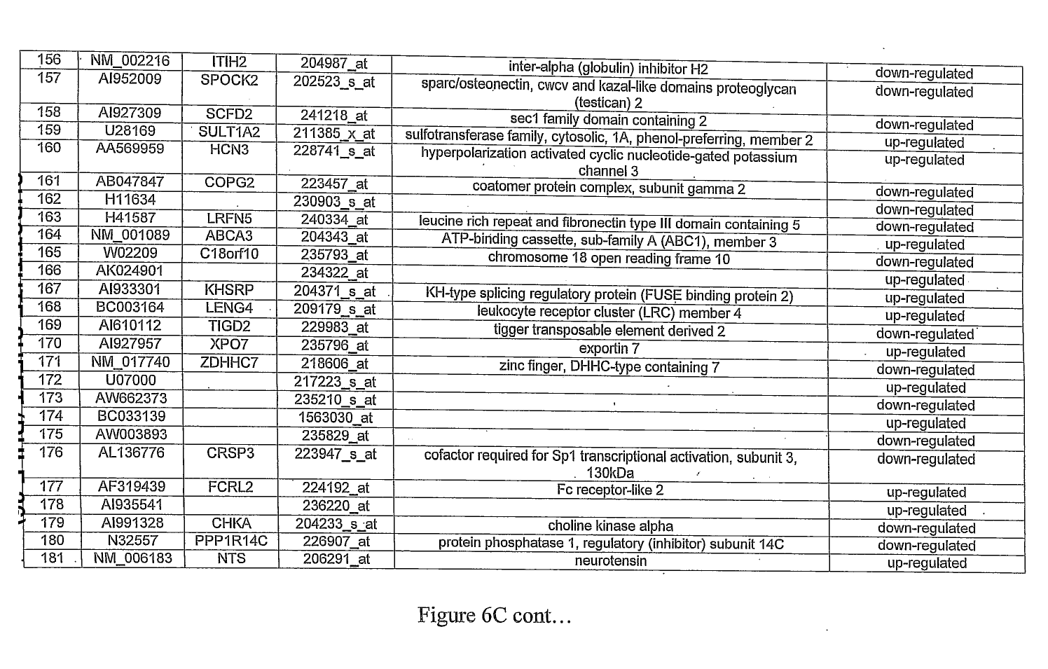

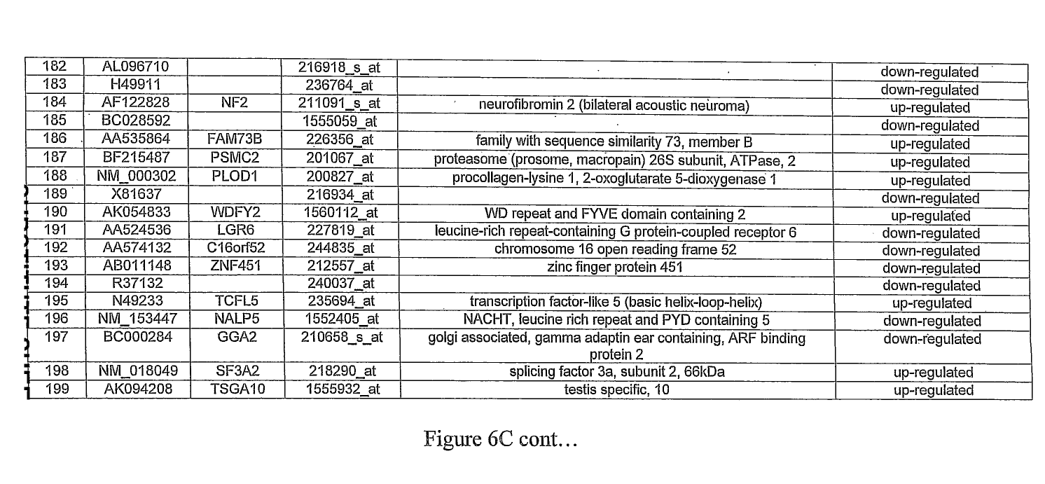

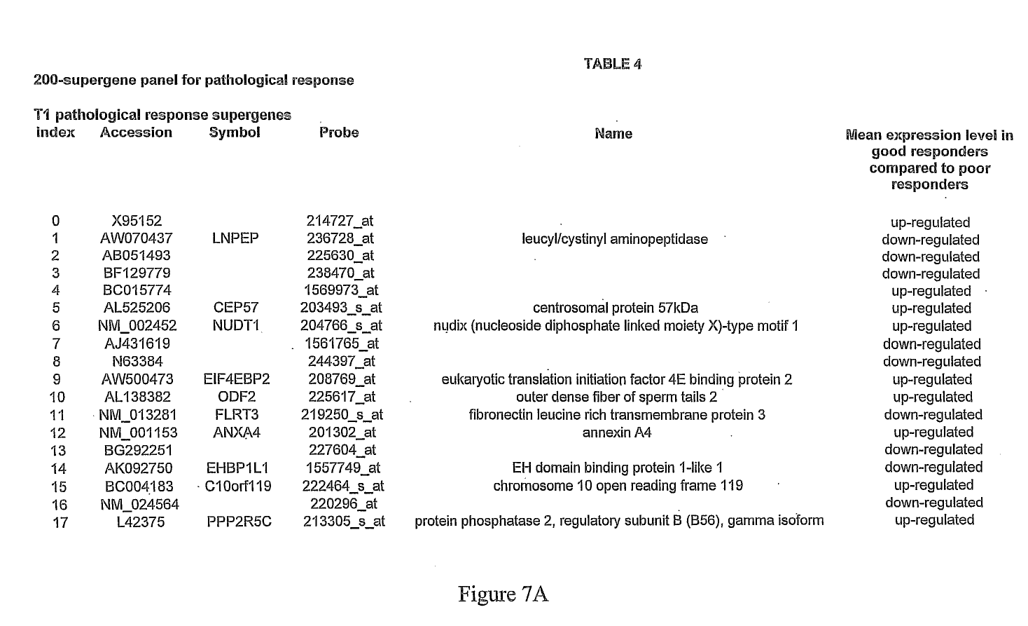

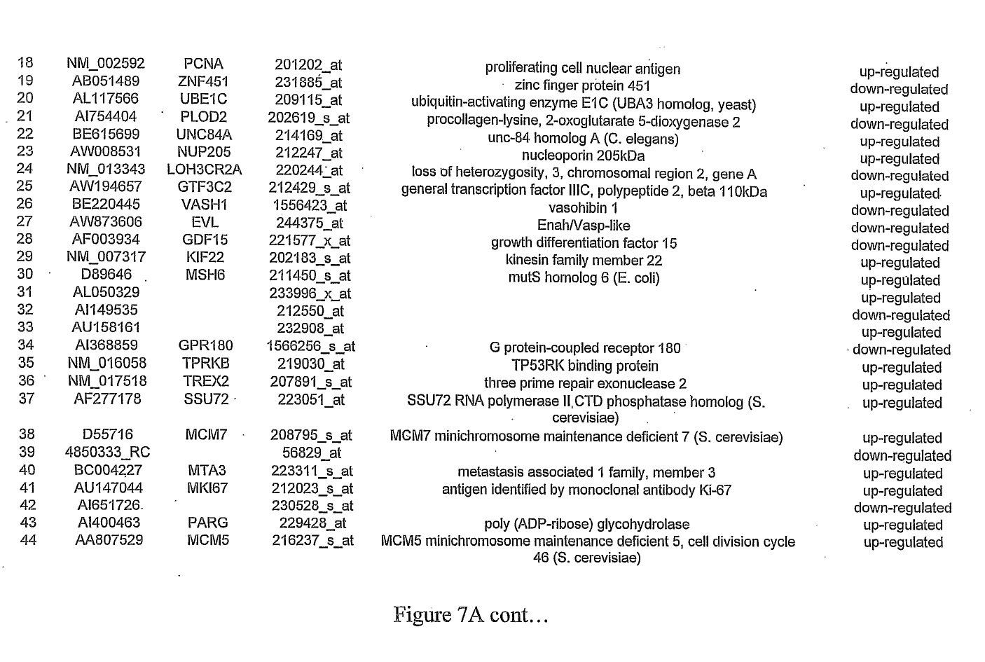

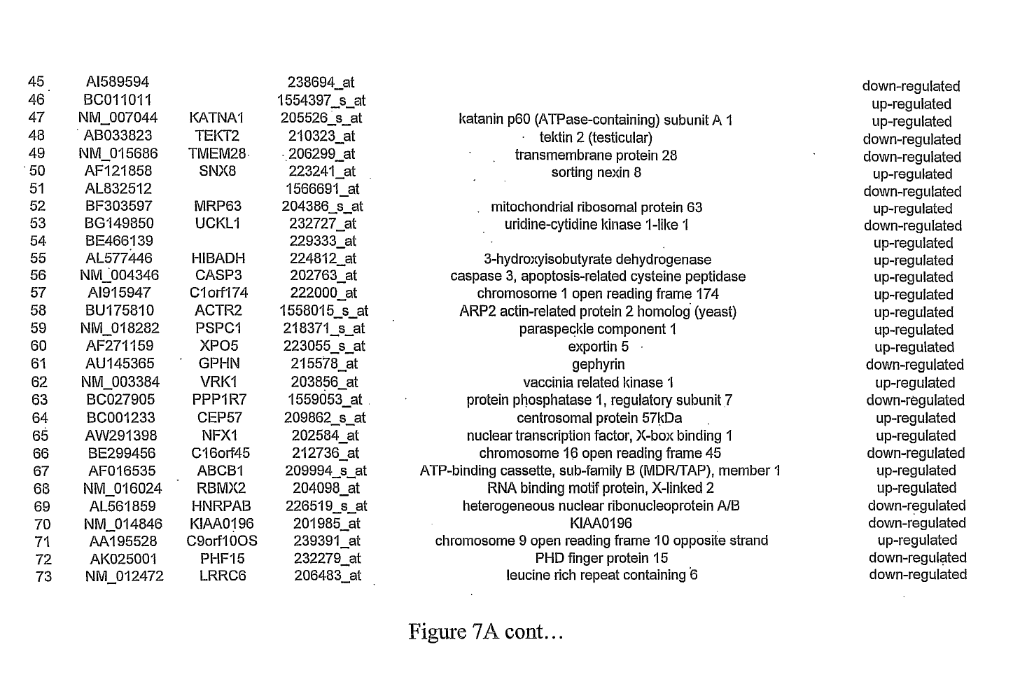

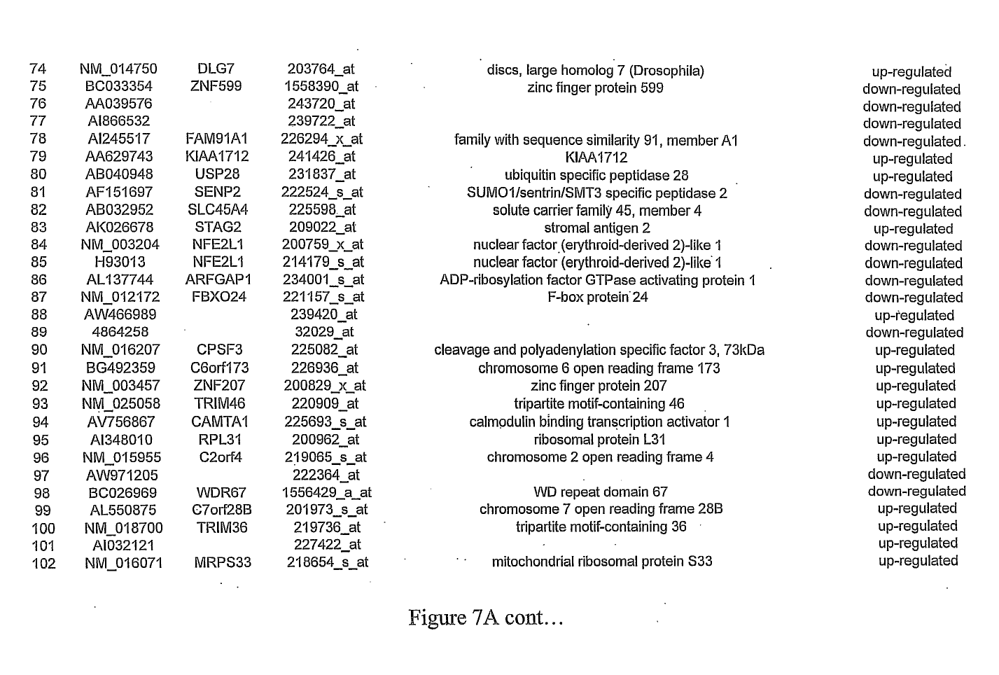

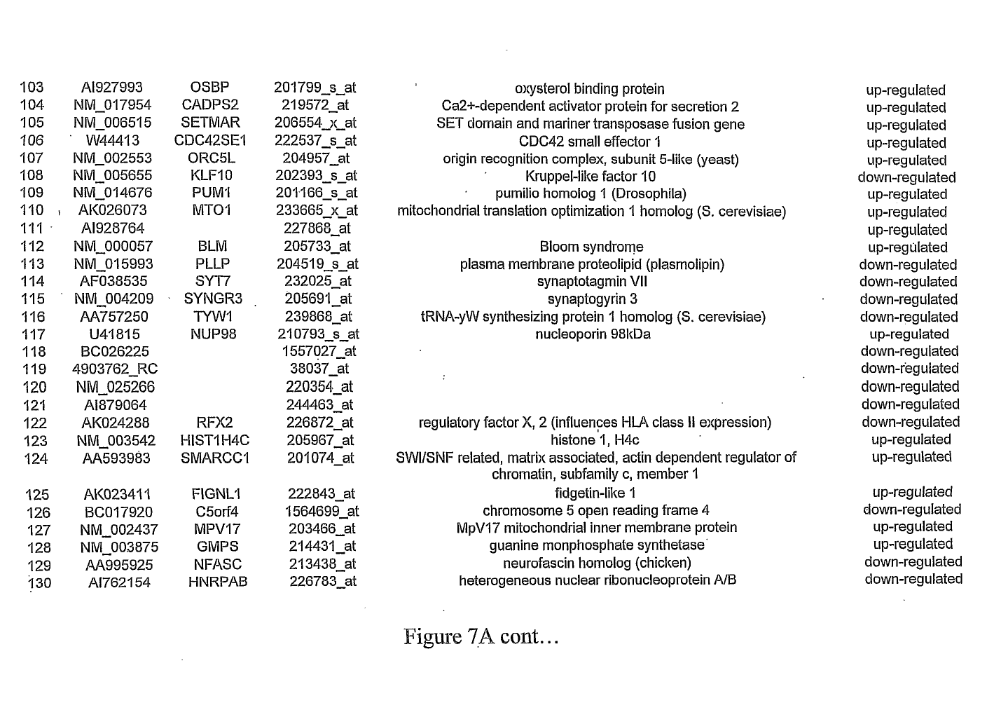

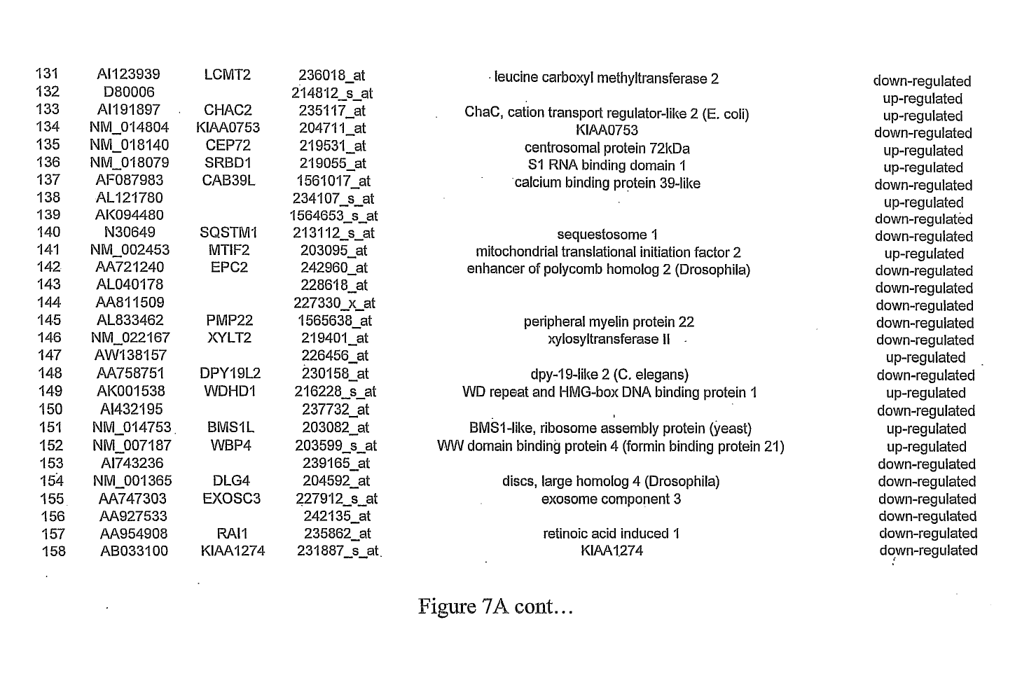

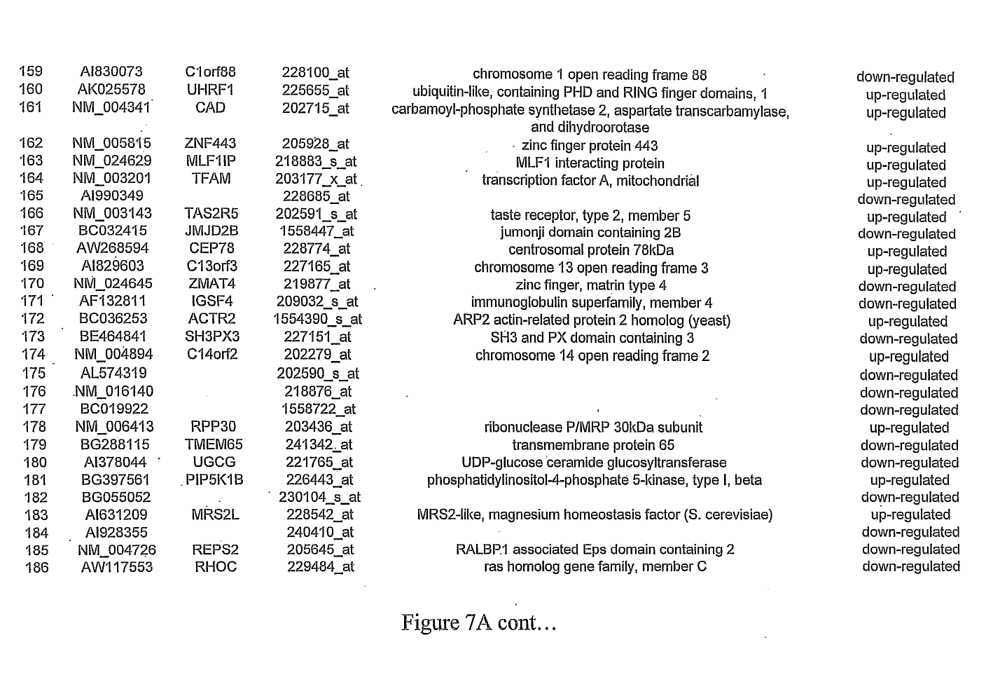

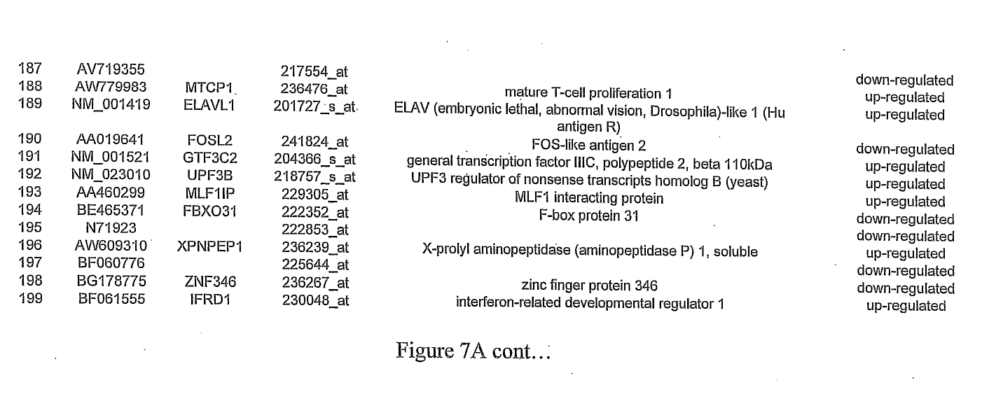

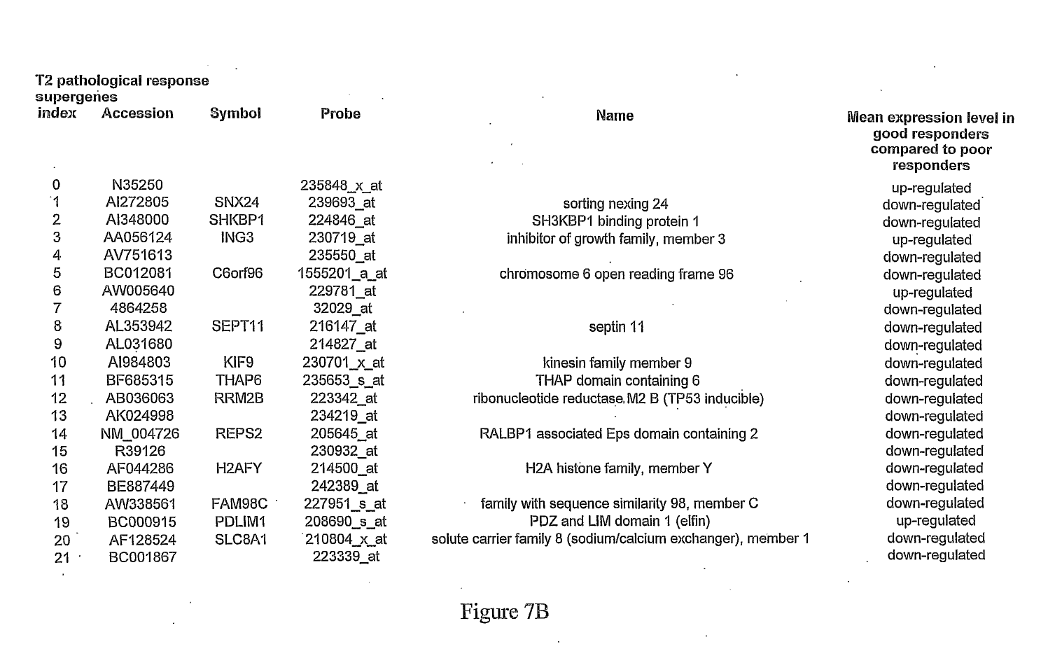

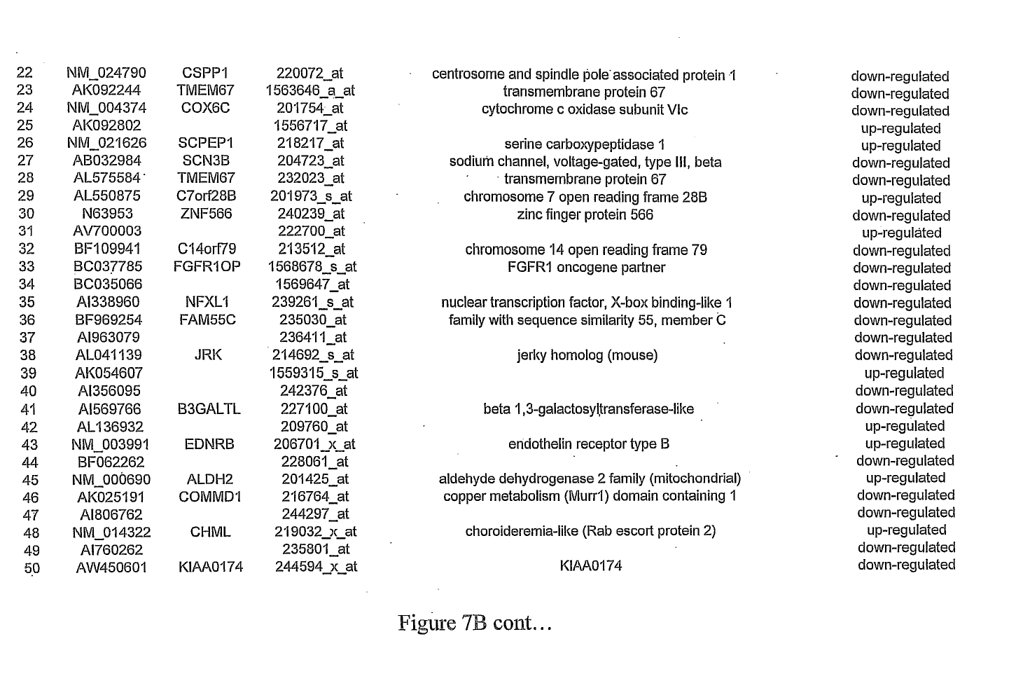

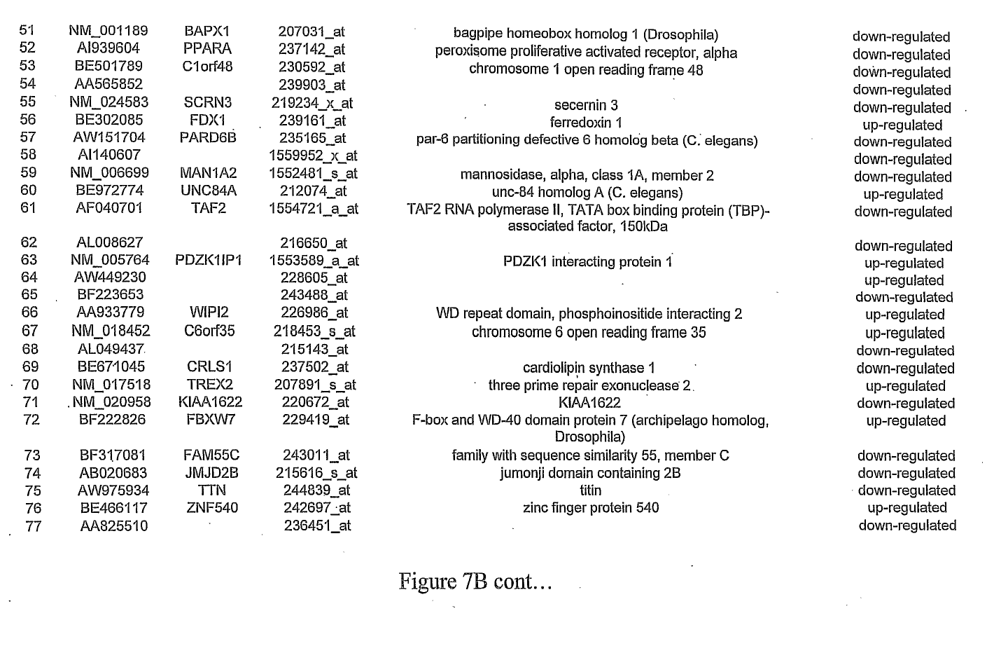

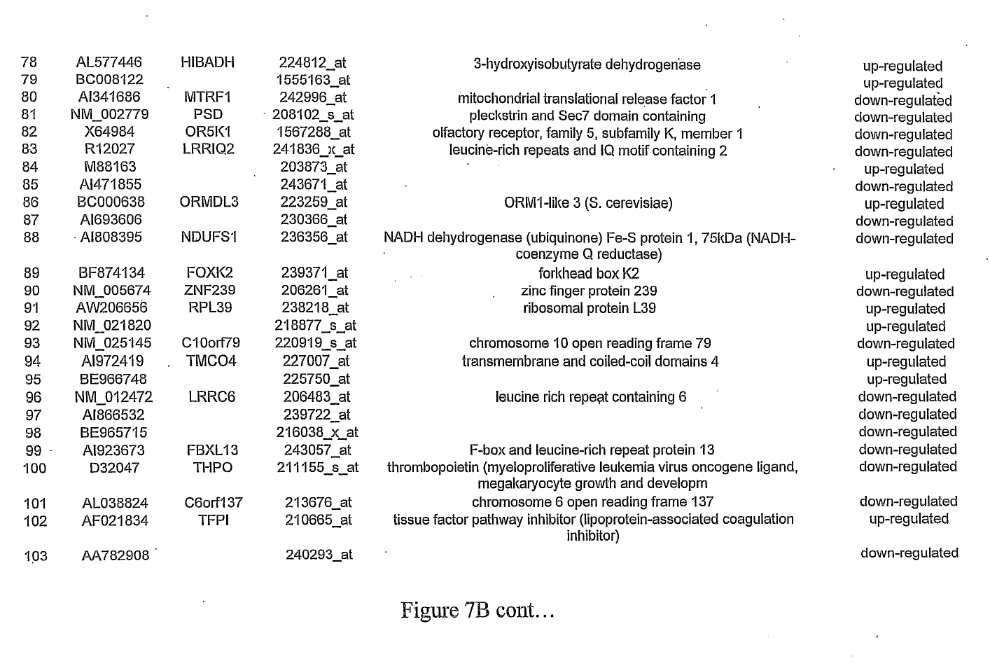

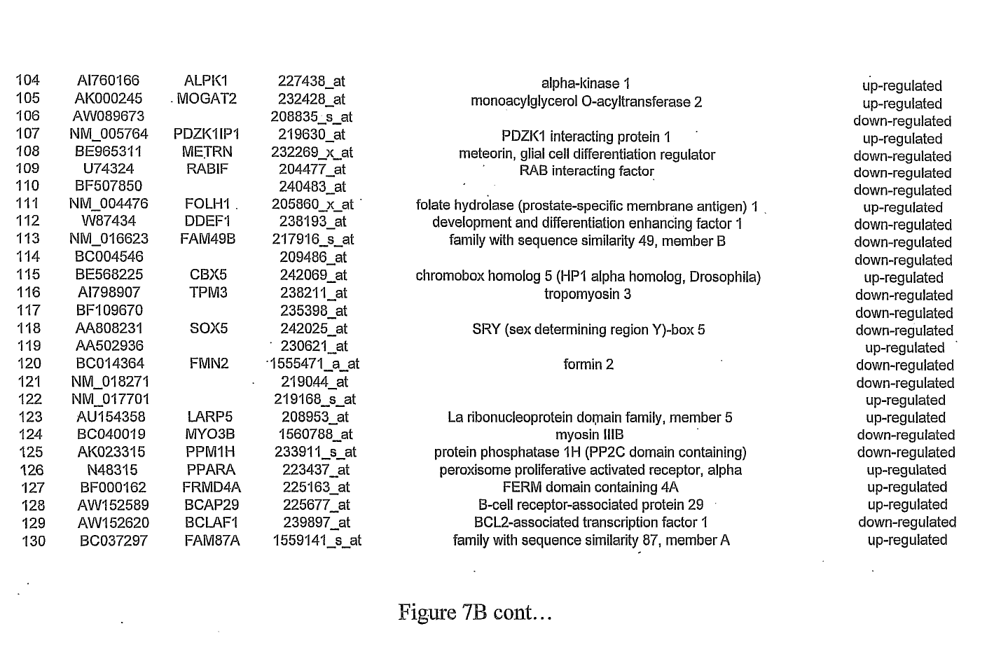

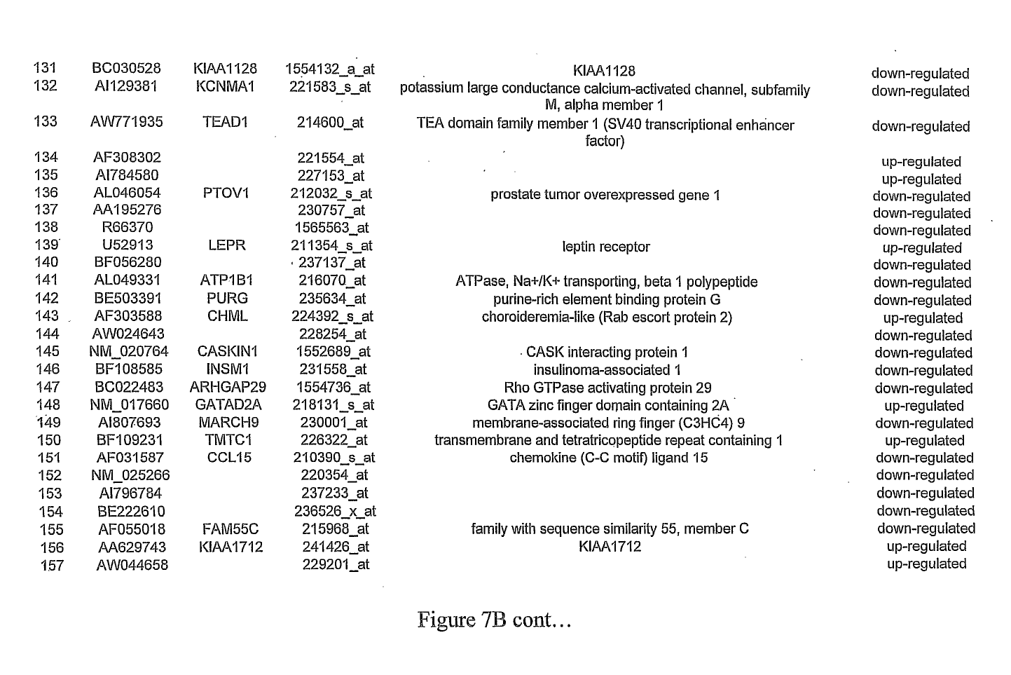

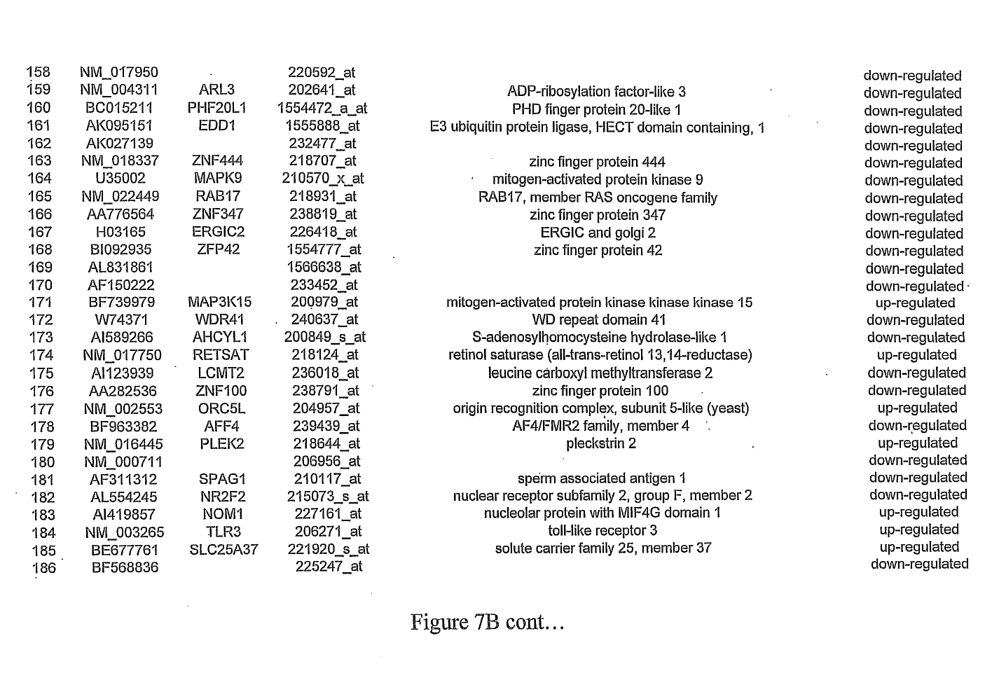

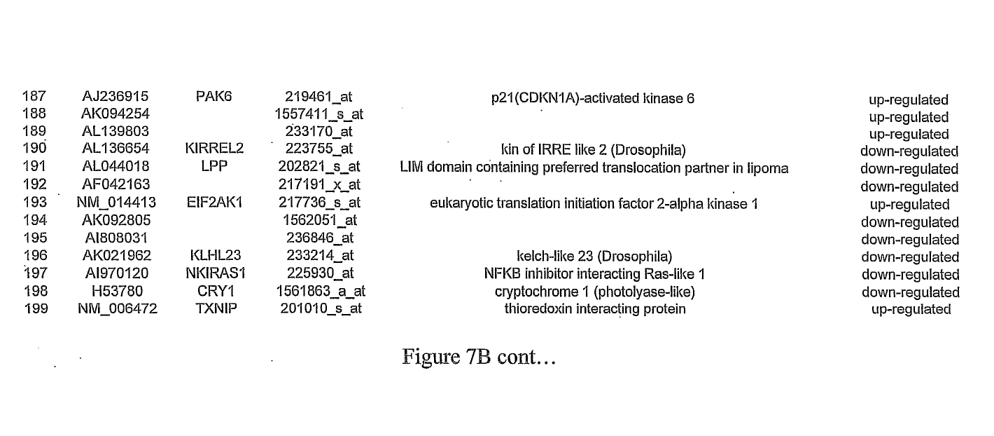

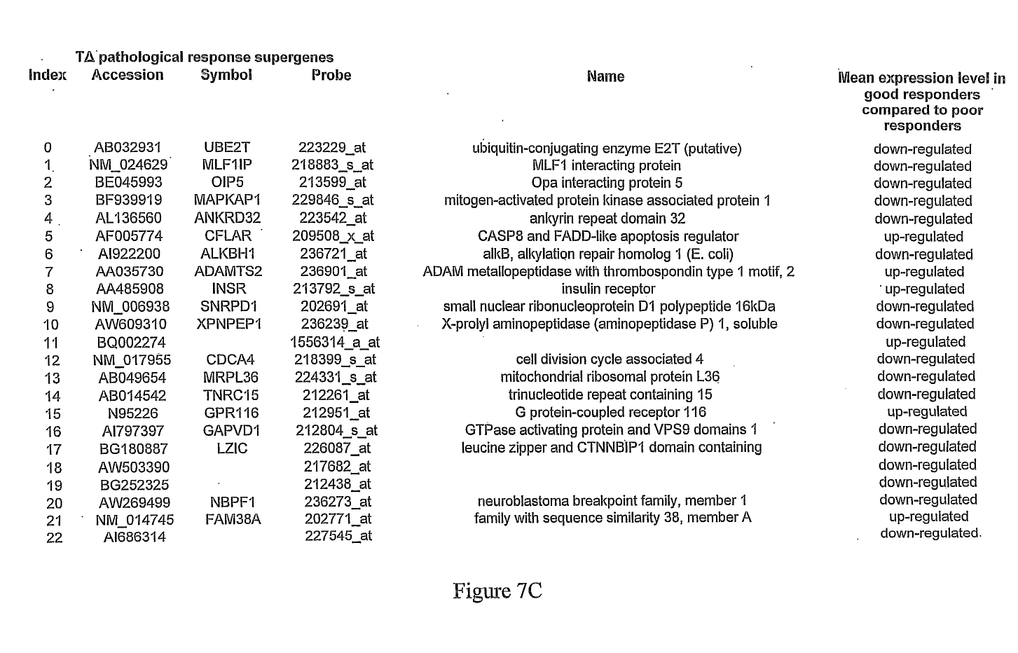

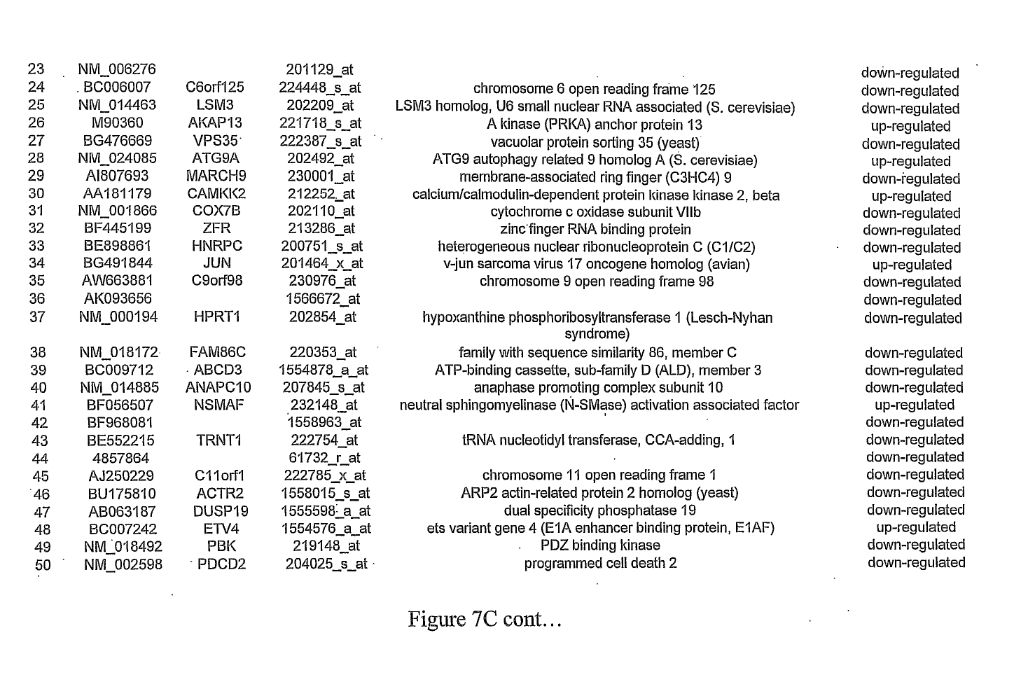

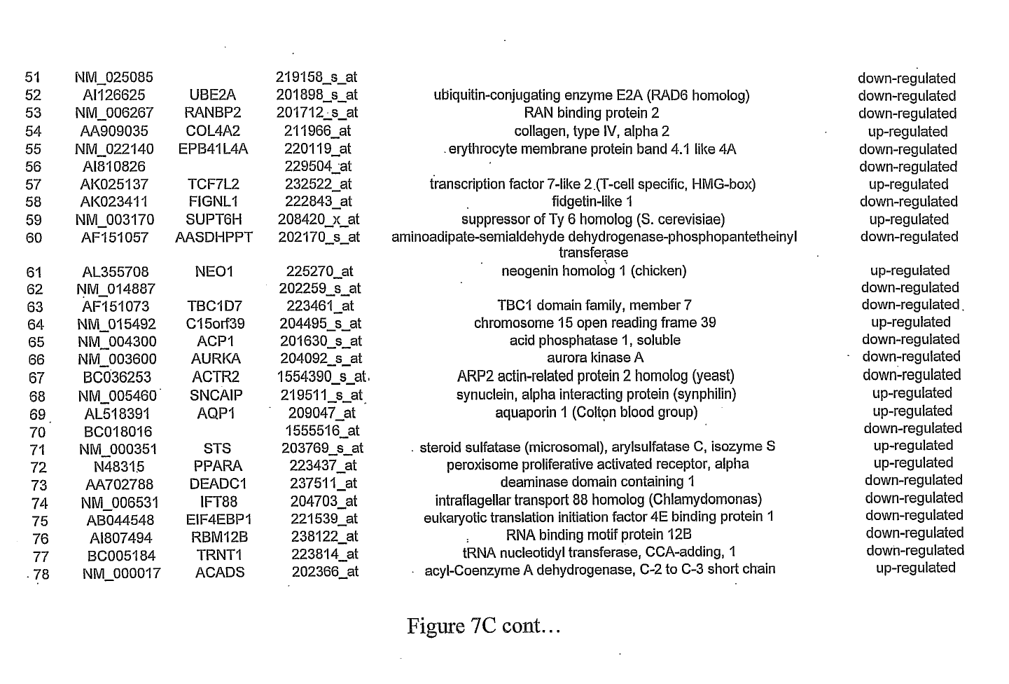

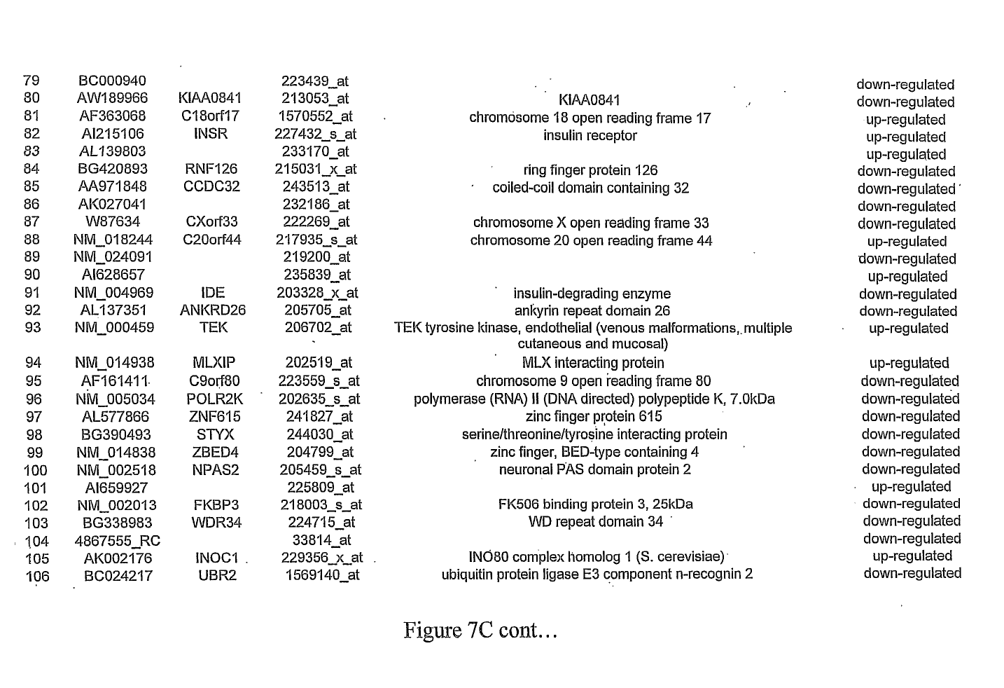

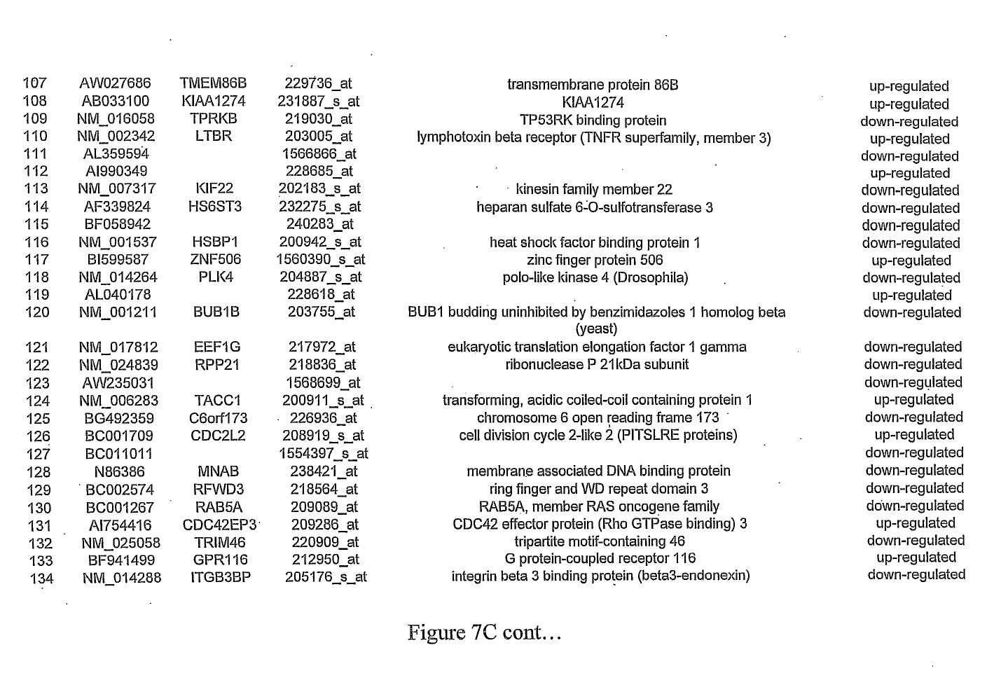

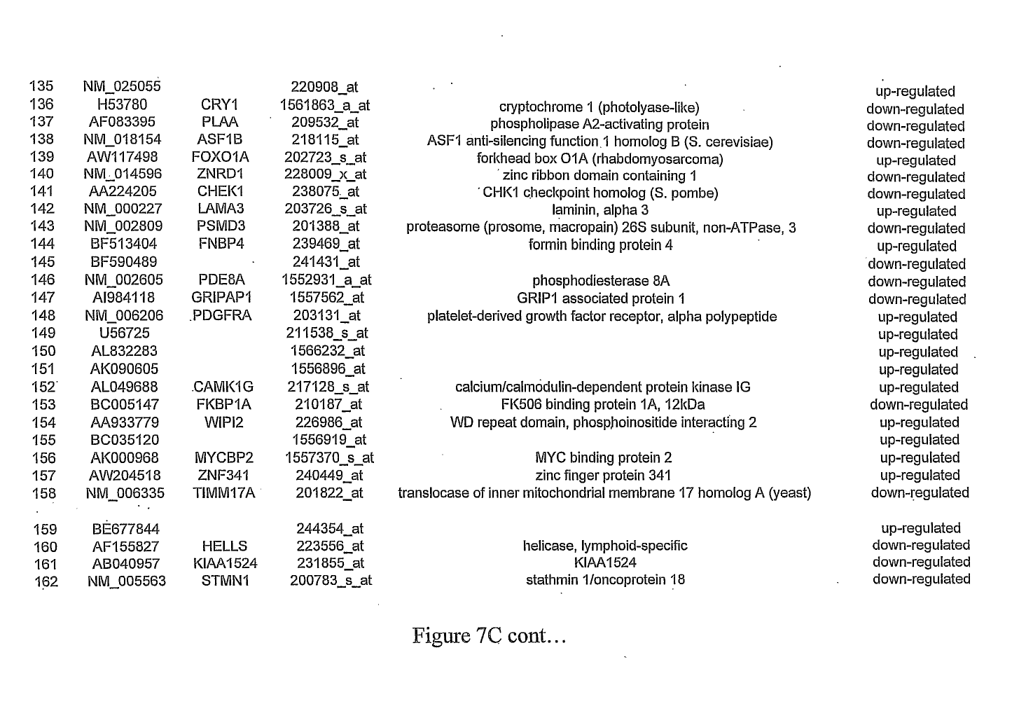

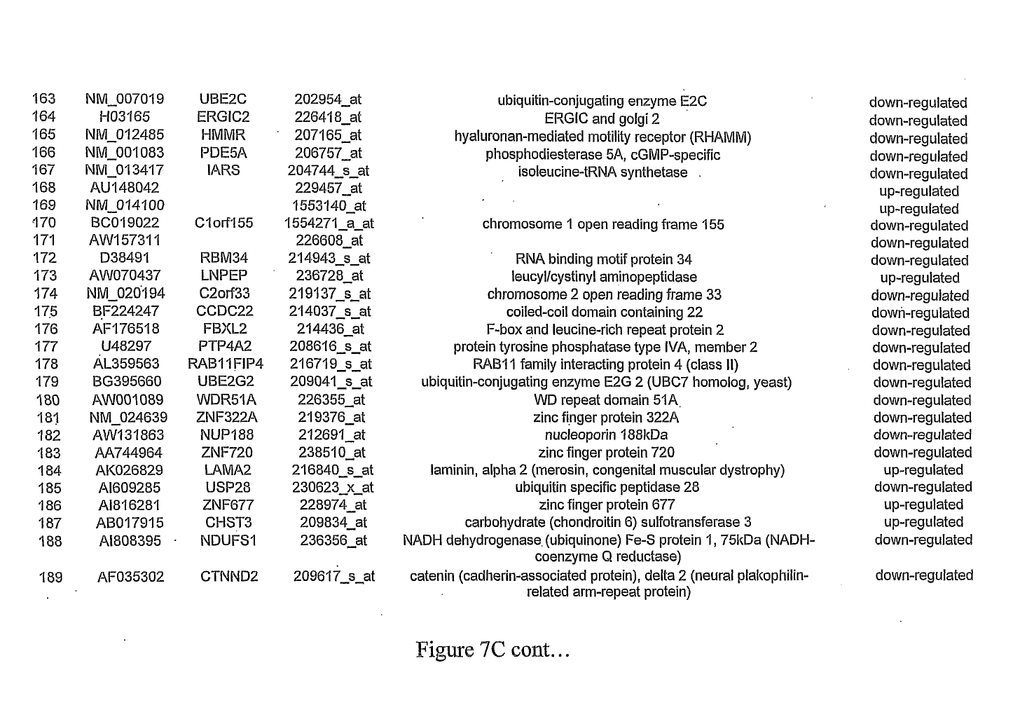

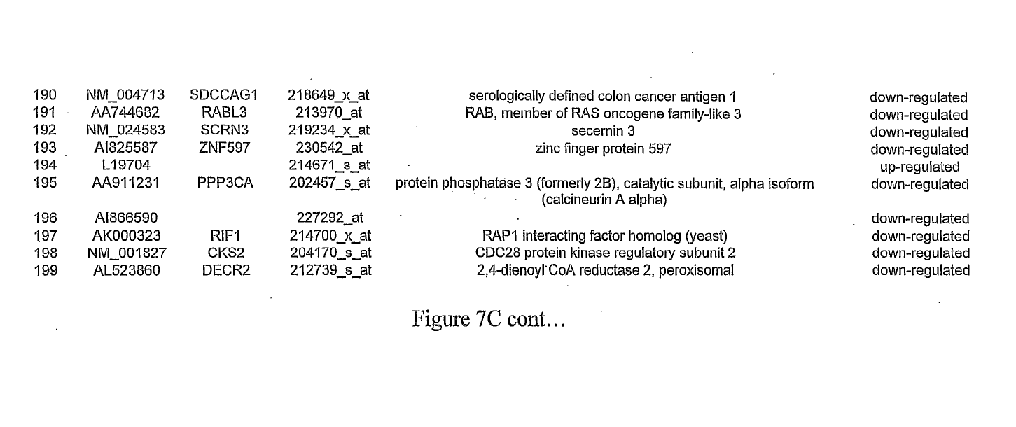

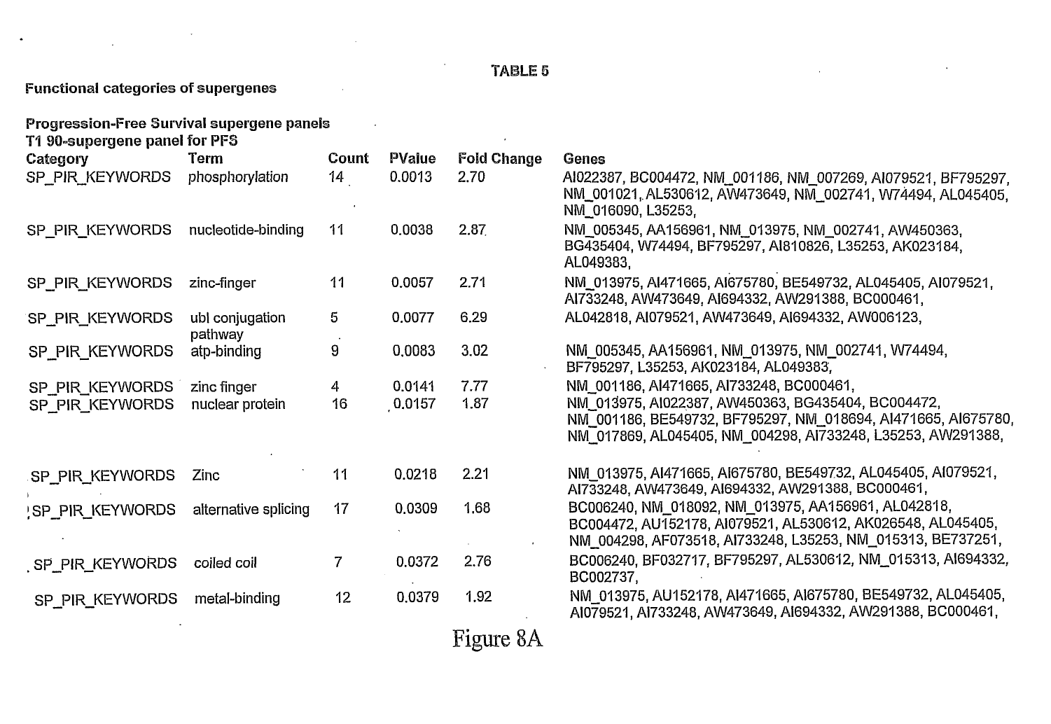

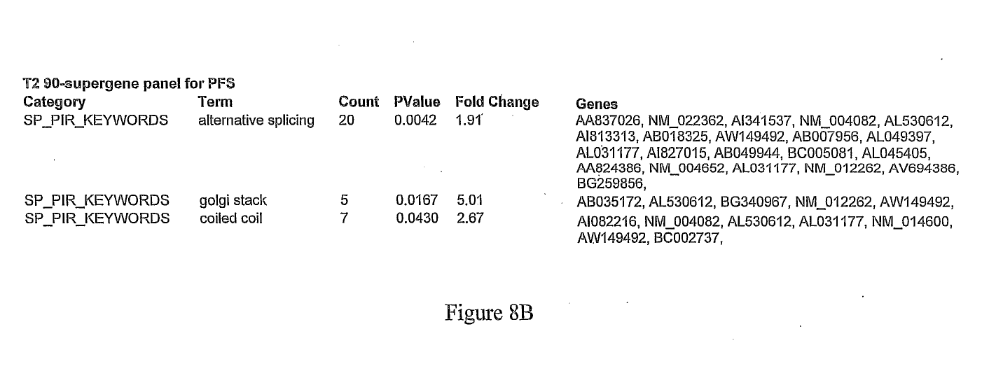

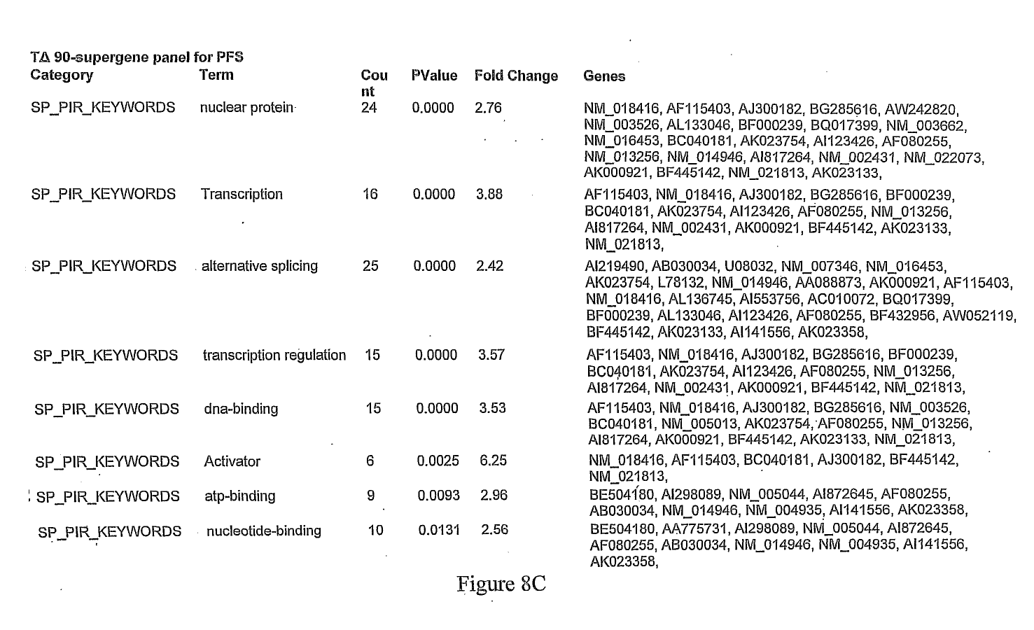

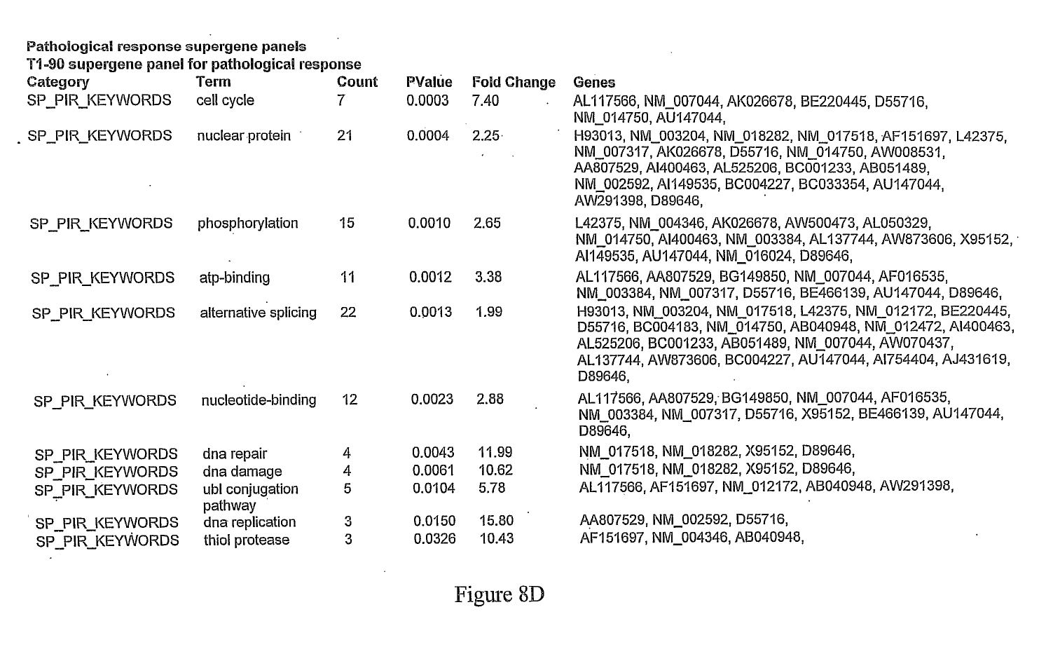

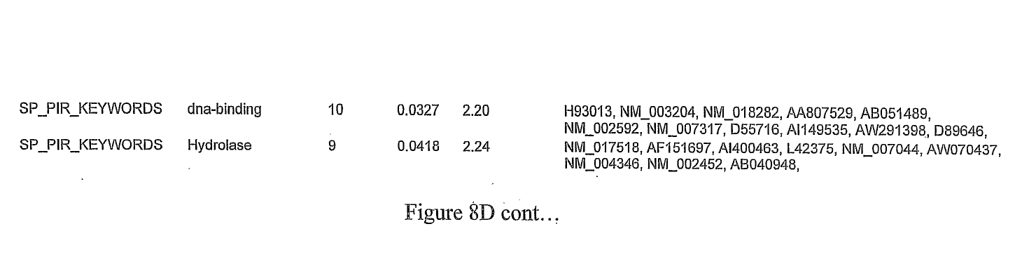

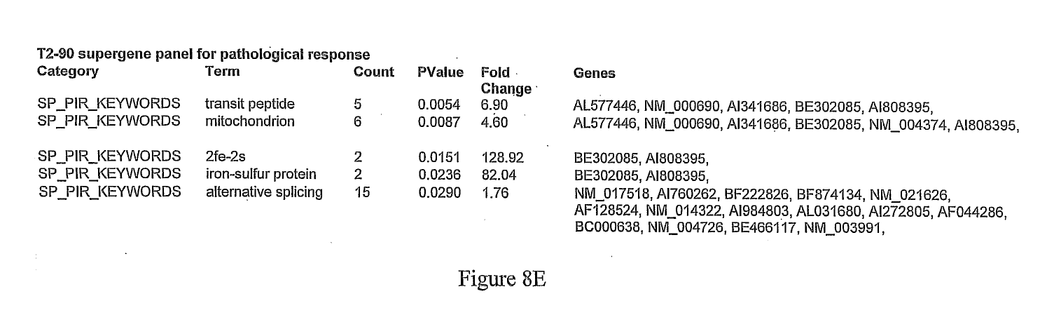

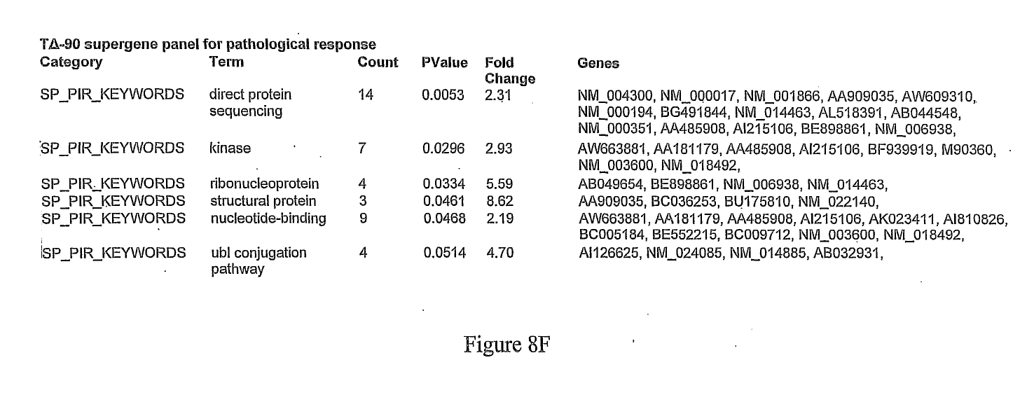

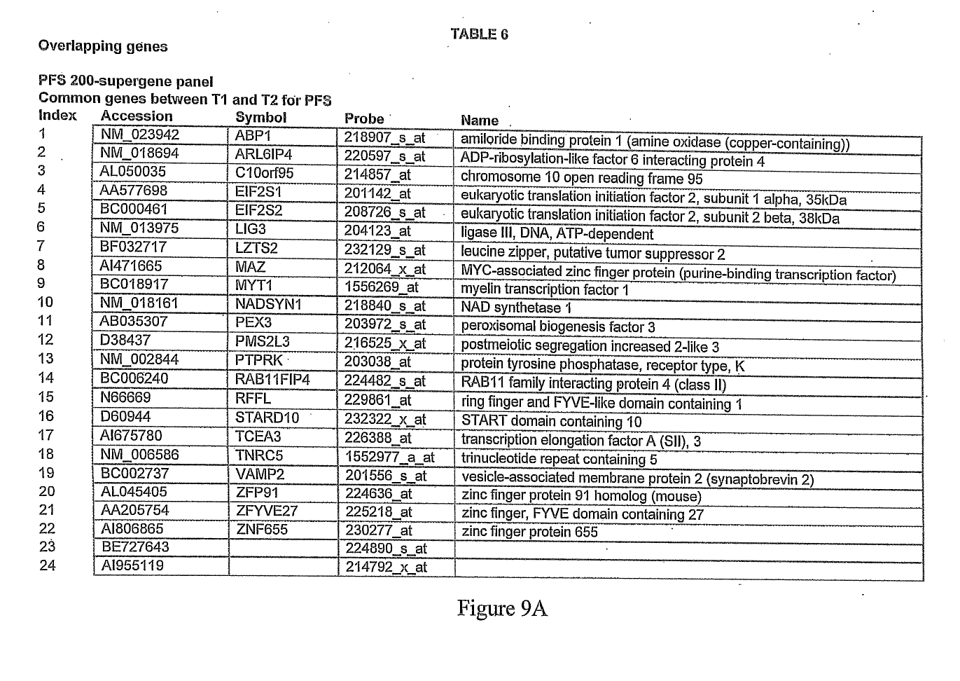

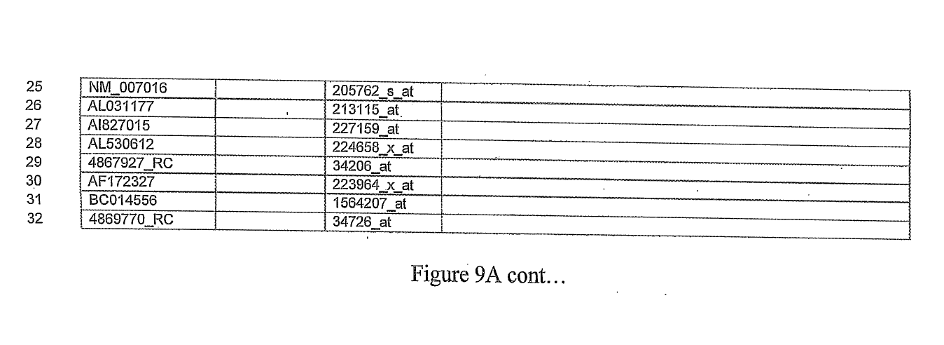

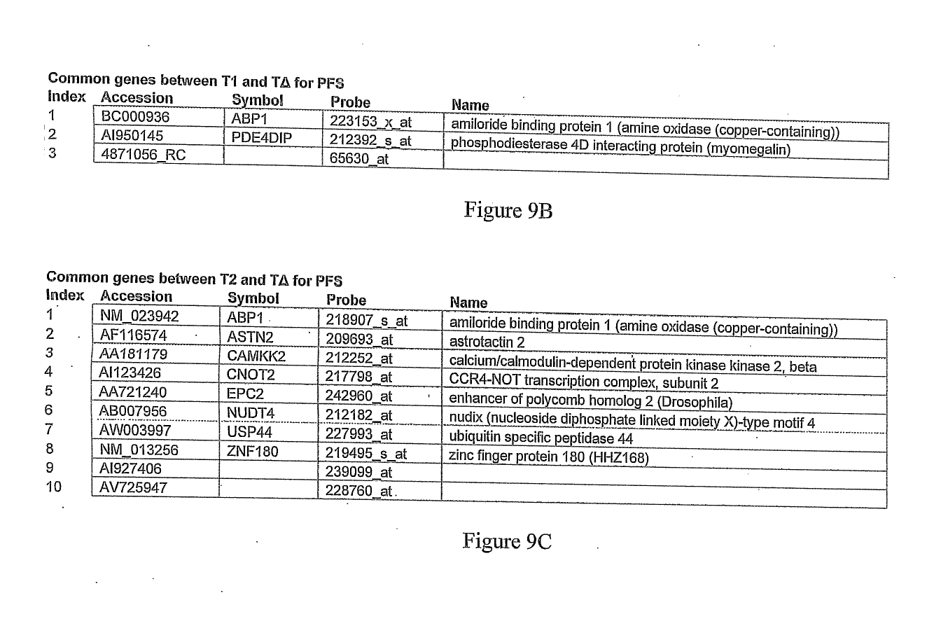

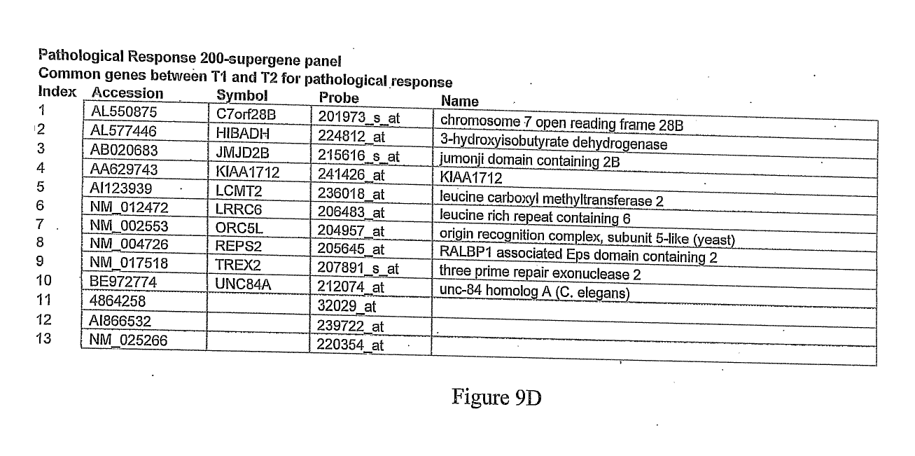

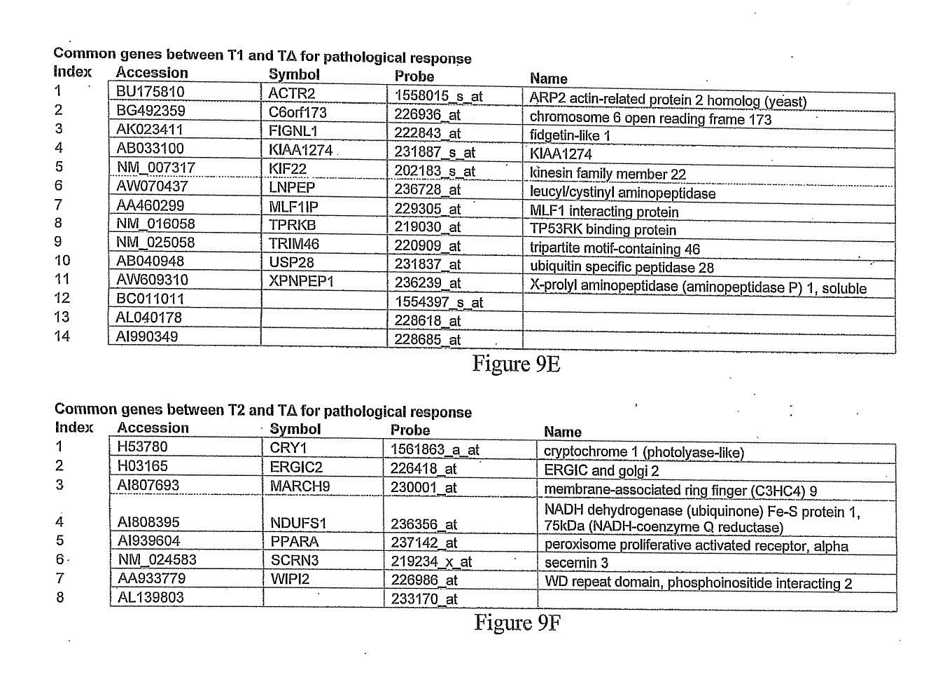

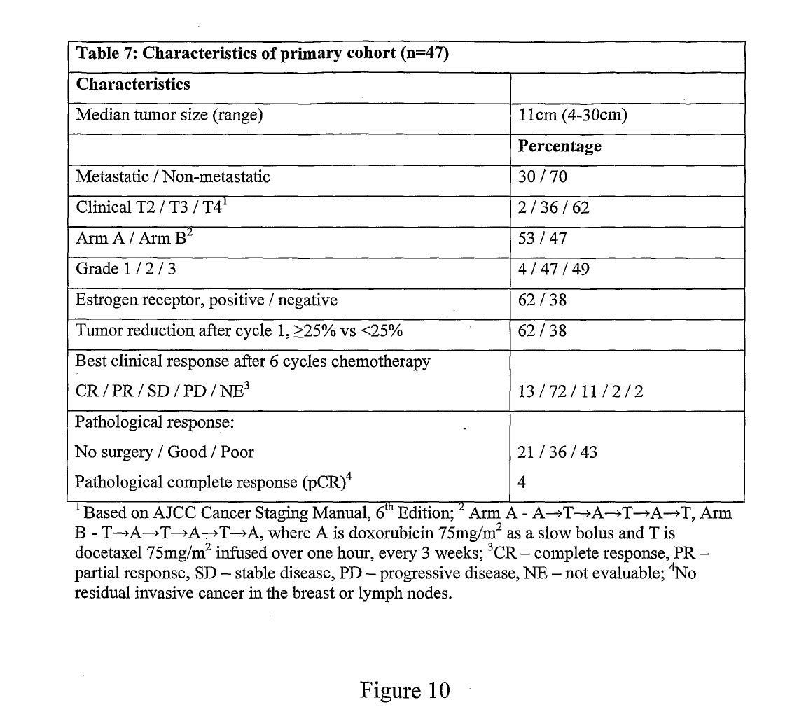

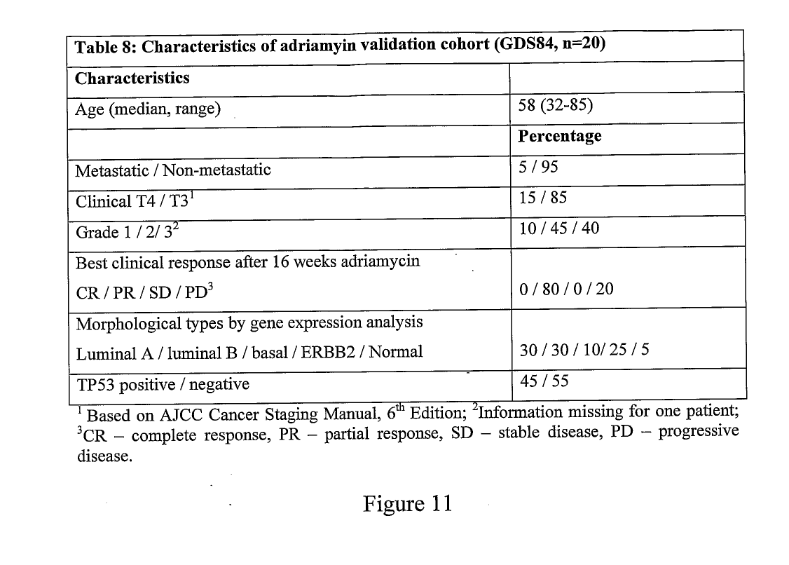

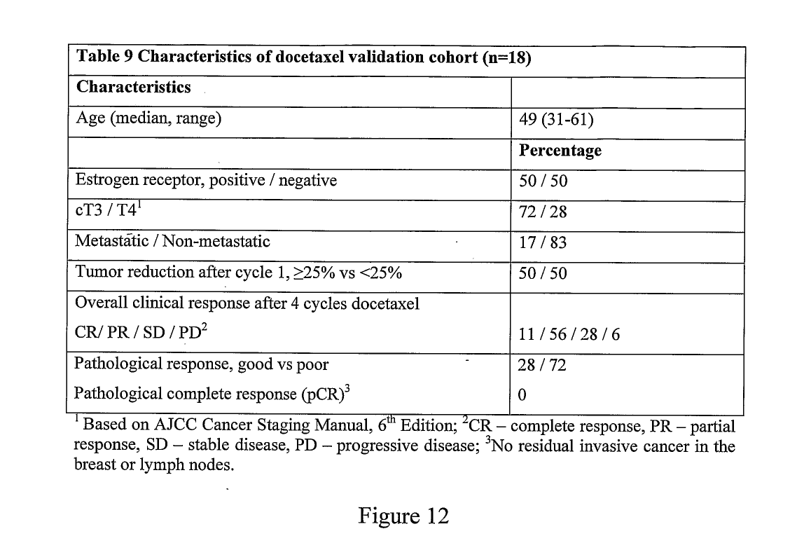

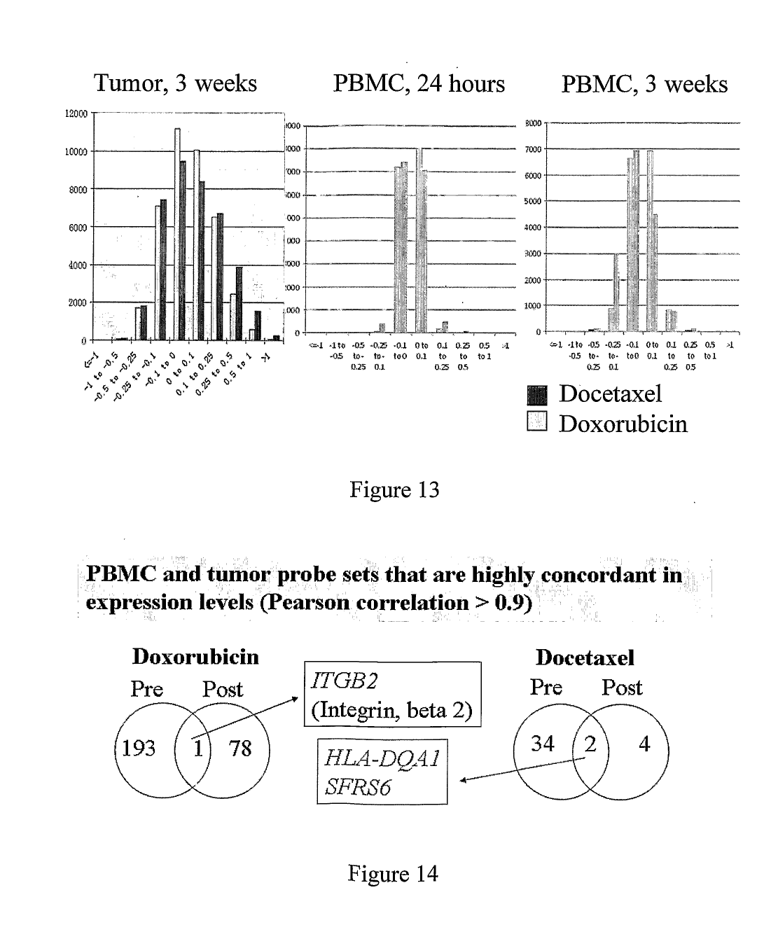

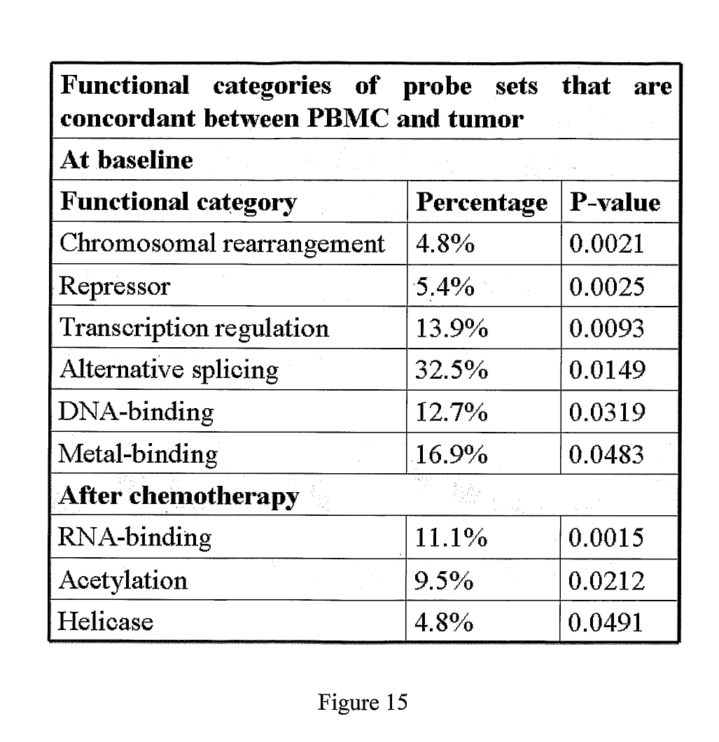

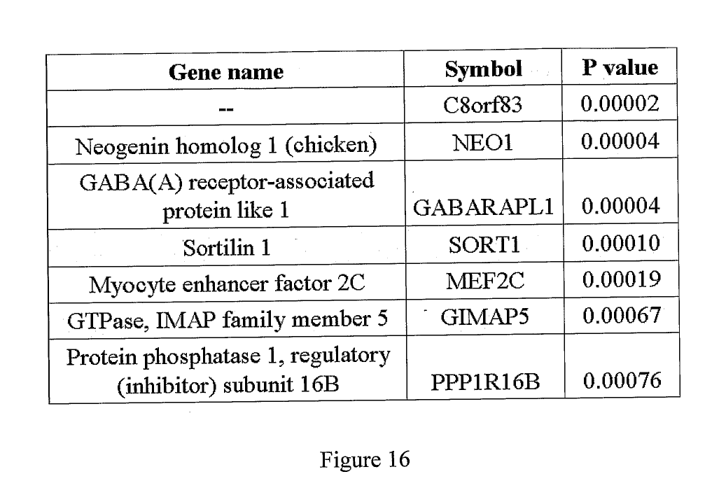

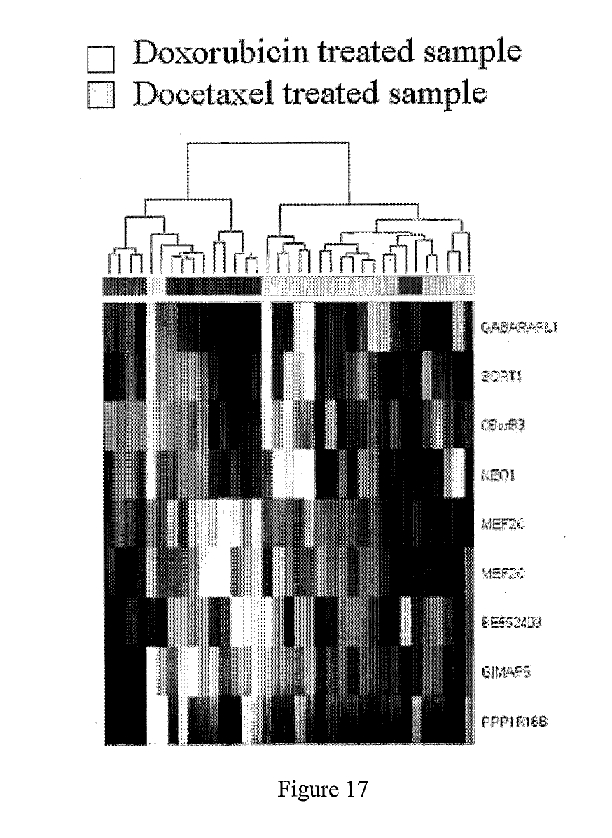

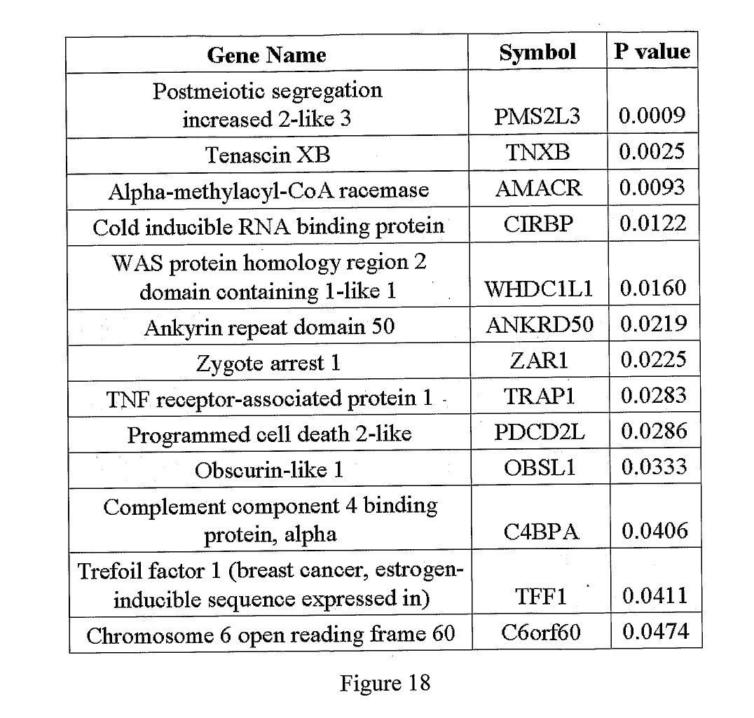

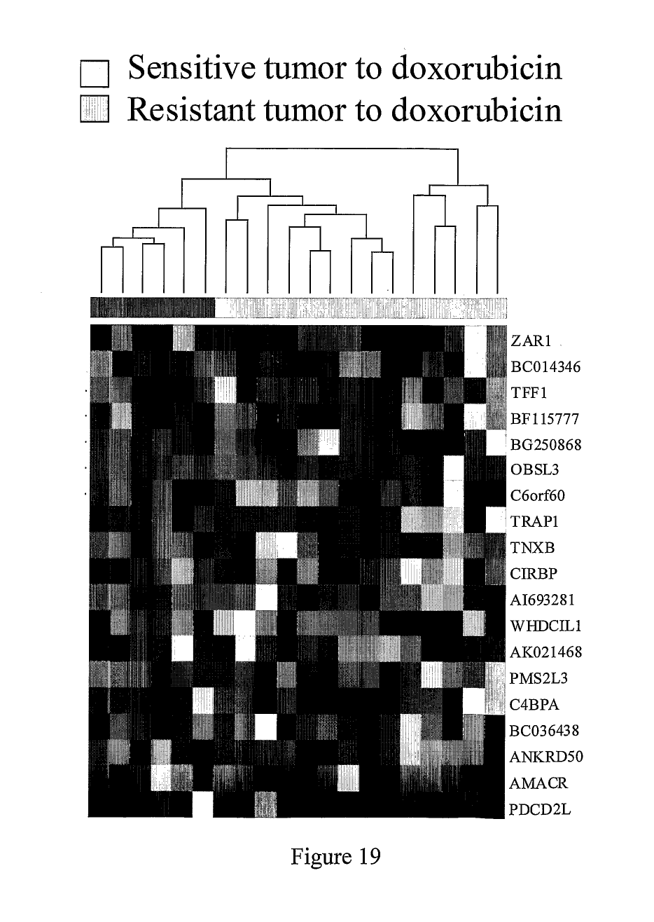

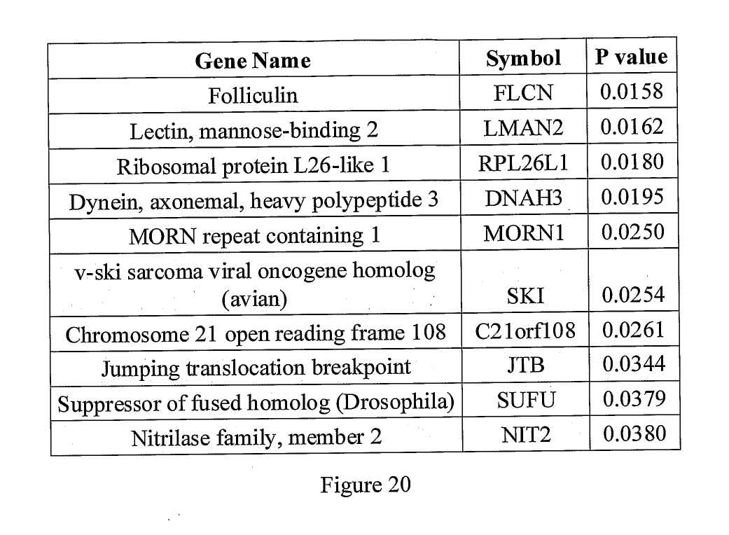

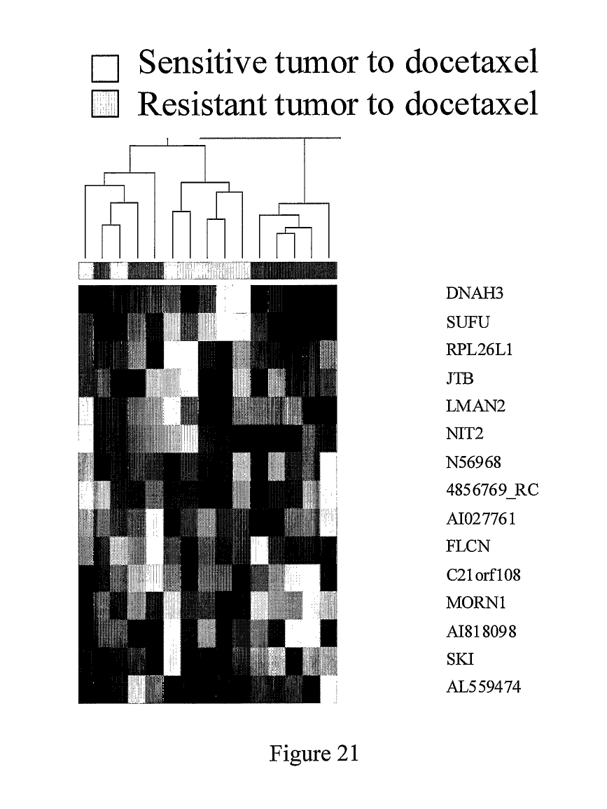

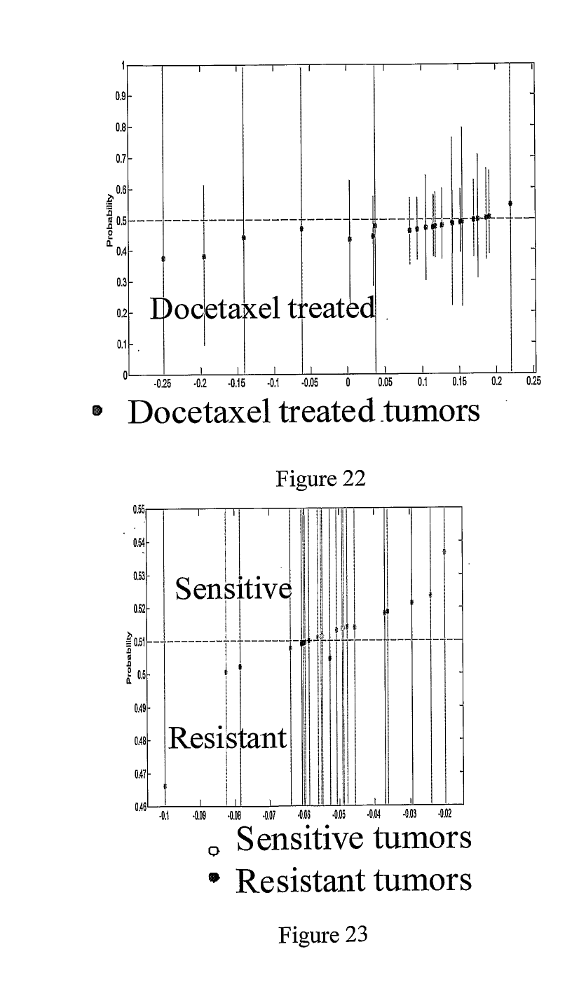

Using unsupervised hierarchical clustering, we applied the three sets of 90-supergene panels for PFS to the original cohort (n=47, FIG. 1a) and compared their performance. As expected, all three panels could distinguish two prognostic groups with high accuracy (p<0.001, FIG. 1b), but both post-chemotherapy signatures were superior to the baseline signatures, with the AUC of the receiver operating characteristic (ROC) curves for T2 and TΔ signatures being 0.893 (95% CI 0.790-0.996, p<0.001) and 1.000 (95% CI 1.000-1.000, p<0.001) respectively, compared to 0.871 for T1 signatures (95%CI 0.759-0.983, p<0.001; FIG. 1c). In multivariate Cox Regression analysis with known clinical prognostic indicators, including presence or absence of metastatic disease, estrogen receptor status, and clinical and pathological response to chemotherapy, only T2 and TΔ signatures and presence or absence of metastatic disease remained significant PFS predictors, while T1 signatures was not (Table 1).

To validate the PFS supergene panels further, we applied them, using support vector machine (SVM), into an independent validation set of 20 patients treated with neoadjuvant weekly, single agent adriamycin and who had post-treatment tumor biopsy after 12 weeks of treatment. The validation set was divided at the median PFS time into short and long survivors. T2 and TΔ were again superior to T1 signatures in predicting for relapse-free survival (RFS, FIG. 2a), with the AUC of the ROC curve for T2 being 0.770 (95% CI 0.558-0.982, p=0.041). The T2 and TΔ signatures separated the cohort into two groups with distinct RFS (FIG. 2b). In multivariate analysis with known clinical prognostic indicators, including pre-treatment tumor stage, clinical nodal status, gene expression morphological subtypes, clinical response to chemotherapy, and tumor TP53 status, only T2 signatures was statistically significant in predicting RFS (p=0.009, Table 2).

Next, we applied the 90-supergene panels for pathological response to the original cohort using unsupervised hierarchical clustering, and found, expectedly, the predictors to be highly accurate; the division yielded groups with different PFS, though not statistically significant (Table 1). To validate the pathological response signatures further, we applied them, using SVM, in an independent set of 18 patients treated with four cycles of neoadjuvant three-weekly single agent docetaxel prior to surgery, who had tumor biopsy before and 3 weeks after one chemotherapy cycle. Pathological response definition was similar to our original cohort. Consistent with the findings for the PFS predictors, T2 outperformed T1 signatures, with the AUC of ROC being 0.631 (95% CI 0.350-0.912) for T2 compared to 0.462 (95% CI 0.190-0.733) and 0.446 (95% Cl 0.161-0.731) for TΔ and T1 respectively. T2 signatures accurately identified 77% of poor pathological responders, compared to 54% for both T1 and TΔ (FIG. 2c-d).

### Methods

Study Population (Table 7) The, primary study population comprised of 47 female patients with histologically or cytologically proven locally advanced or metastatic breast cancer who have measurable primary breast tumors and who were receiving systemic chemotherapy before surgery. All subjects provided written informed consent, and the study protocol was approved by the Institutional Ethics Committee. Subjects were randomized to receive one of two alternating sequences of doxorubicin (A) or docetaxel (T), starting either with doxorubicin 75 mg/m² as a slow bolus or docetaxel 75 mg/m² infused over one hour every 3 weeks for 6 cycles (A→T→A→T→A→T, n=25; or T→A→T→A→T→A, n=22). Tumor assessments were performed at every cycle with bi-dimensional tumor measurements with a caliper and using the WHO criteria for tumor response.

Patients were classified to have intrinsically sensitive or intrinsically resistant tumors to the drug that they received in the first cycle if they achieved ≧25% or <25% reduction in tumor dimensions respectively after the first cycle of chemotherapy. After six cycles of chemotherapy, patients were classified as responders if they achieved complete response (no clinically measurable disease) or partial response (>50% reduction in tumor dimensions), and as non-responders if they achieved stable disease or progressive disease as the best response, according to the WHO criteria.

Of the 47 patients in the primary cohort, 37 underwent lumpectomy or mastectomy with axillary lymph node clearance and were evaluable for pathological response. They were ranked according to their pathological tumor size and nodal status, and an arbitrary division was drawn at approximately the median to yield two groups, good and poor pathological responders, of comparable sizes. With this division, patients were deemed to be good pathological responders if they had residual tumor measuring no more than 5 cm and had no more than one axillary lymph node involved with tumor (17/37 patients, 46%). Patients were considered poor pathological responders if they had residual tumor measuring more than 5 cm or had two or more axillary lymph nodes involved with tumor (20/37 patients, 54%). Using this definition, poor pathological responders were found to have shorter progression-free survival (median 40.7 months, range 19.5-62 months) than good pathological responders (median PFS not reached), although the difference was not statistically significant because of small sample size (p=0.224). However, when the same pathological response definition was applied to a larger cohort of 73 patients treated in the same protocol, the difference was statistically significant (median PFS for poor pathological responders 30.3 months, range 9.3-51.5 months, median PFS for good pathological responders not reached; p=0.002), suggesting the division to be clinically relevant.

Progression-free survival (PFS) was defined as the time between date of randomization and the first documented evidence of relapse, progression, or death, whichever occurred first. For patients who are still alive at the time of analysis and who have not had documented disease progression, PFS was censored at the date of the last follow-up visit. At the time of analysis, the median follow-up of the cohort was 36 months, and 23/47 patients had progressed. After stratifying for presence or absence of metastatic disease, the sample was divided at the median PFS time into short and long survivors for the purpose of deriving predictive gene panels for PFS in the training sets.

### Gene Expression Analysis

Serial tumor core biopsies were taken from each subject's primary breast tumor before the first cycle of chemotherapy and about 3 weeks later but before the second cycle of chemotherapy. Tumor cores were snap frozen in liquid nitrogen and stored at −80° C. until analysis. Messenger RNA was extracted from breast tumor core biopsies using RNeasy mini kit (Qiagen, Venlo, The Netherlands), labeled by biotin, and hybridized on the Affymetrix U133+2 microarray chip (Santa Clara, Calif.) comprising of 54,675 probe sets corresponding to 38,500 genes, according to the manufacturer's instructions (Santa Clara, Calif., USA). Scans were performed with an Affymetrix GeneChip scanner and the expression value for each gene was calculated using the Affymetrix Microarray Analysis suite (v5.0), computing the expression intensities in arbitrary ‘signal’ units defined by the software. Scaling factors were determined for each hybridization based on an arbitrary target intensity of 500. Scans were rejected if the scaling factor exceeded a factor of 30. Expression was calculated using the robust multi-array average (RMA) algorithm implemented in the Bioconductor (http://www.bioconductor.org) extensions to the R statistical programming environment. RMA generates log-2 scaled measures of expression using a linear model robustly fit to background-corrected and quantile-normalized probe-level expression data.

### Statistical Analysis

We performed analysis on 39,660 probe sets after filtering out 15,015 probe sets that were detectable in fewer than five experiments from the initial 54,675 probe sets. Independent t-tests were performed to select genes that were differentially expressed by good and poor pathological responders, and by long and short survivors. Gene expression signatures of the baseline tumor (T1), post-chemotherapy tumor (T2) and the relative changes after chemotherapy (TΔ) were compared with respect to their ability to predict for pathological response and progression-free survival. Predictive gene panels were derived by randomly dividing the cohort into training and test sets to identify top discriminating genes and refining the gene panels by re-sampling the dataset 10,000 times. In each bootstrap, an independent t-test is applied to the training set to compare the good and poor response or prognostic groups, and a p-value computed for each probe set that is detectable in at least 5 experiments. With each cumulative bootstrap, a cumulative p-value is calculated for each probe set. The final weight for each probe set is defined as the inverse of the sum of the p-values for the total number of bootstraps performed, with probe sets having greater weights being ranked more highly. The final list of top 200 supergenes was obtained based on the final weights of probe sets after completing 3000 bootstraps. Gene functional classification was performed using the Database for Annotation, Visualization and Integrated Discovery (DAVID) 2007 tool suite available on http://david.abcc.ncifcrf.gov/.

### Validation of 90-Supergene Panel in Original Cohort

Predictive panels comprising of the top 90 supergenes for T1, T2, and TΔ were applied into the entire primary cohort (n=47) using unsupervised cluster analysis with the Gene Cluster software, version 3.0 and the Treeview Program (Stanford University). Kaplan-Meier curves were plotted for each set of predictive gene panel, and the log rank test applied to compare the prediction groups. Receiver operating characteristic curves were constructed to compare the performance of the three sets of gene panels. Univariate analysis was performed to identify clinical and genetic predictors of progression-free survival. Factors that had p-values of ≦0.10 on univariate analysis were applied in multivariate analysis with Cox Regression analysis for progression-free survival. All statistical analyses were performed using the SPSS statistical software, version 13.

### Validation Dataset for Progression-Free Survival (Table 8)

An independent dataset available on the NCBI Gene Expression Omnibus website (GDS84) comprising of 20 locally advanced breast cancer patients treated with single agent doxorubicin was used to validate the signatures that predict for progression-free survival¹⁴. In brief, patients with locally advanced breast cancers were treated with weekly doxorubicin at 14 mg/m² for 16 weeks, and tumor biopsies were taken before chemotherapy and after 16 weeks of treatment for gene expression analysis. Gene expression analysis was performed using cDNA arrays with 9216 probe sets produced at the Stanford Functional Genomics Facility. Using gene symbol mapping, 3440 overlapping gene symbols between the cDNA array and the Affymetrix U133+2 gene chip array were identified and used for analysis. 12/20 patients had relapsed, and the median relapse-free survival was 25 months. The top 90 supergenes for T1, T2, and TΔ were applied into this dataset using support vector machine (SVM). In brief, long and short survivors were denoted +1 and −1 respectively. After training in the original cohort of 47 patients, SVM is applied to the test set where it assigns a predicted value for each test sample based on the expression values of the 90 supergenes, with the sign of the value indicating the predicted class, positive indicating class +1 and negative indicating class −1. The absolute SVM value corresponds to the probability of a test sample belonging to a certain class, with the probability increasing with increasing absolute value. Kaplan Meier curves were plotted and the log rank test performed to determine the quality of the predictive gene panels.

### Validation Dataset for Pathological Response (Table 9)

An independent dataset comprising of 18 breast cancer patients treated with pre-operative chemotherapy followed by surgery as part of another clinical protocol at our institution was used to validate the gene expression signatures that predict for pathological response. Validation was performed using SVM. In brief, patients from this independent dataset had cT3-T4 breast tumors and were treated with four cycles of pre-operative single agent docetaxel every three weeks. Pre- and post-treatment tumor biopsies approximately 3 weeks after the first chemotherapy cycle were obtained for gene expression analysis on the Affymetrix U133+2 array. Patients underwent lumpectomy or mastectomy and axillary clearance after chemotherapy, and were classified as good (5/18) and poor (13/18) pathological responders using the same criteria as described in our primary cohort. This dataset was not used to validate the PFS signatures as the median follow-up was short at 18.5 months, and only 5/18 had progressed at the time of analysis.

### Discussion

Gene expression signatures of untreated tumor have been used to classify¹, prognosticate²⁻⁵ and predict chemotherapy sensitivity⁶⁻¹⁰ with great promise and enthusiasm. In contrast, relatively little attention has been paid to post-treatment tumor signatures and their relation with treatment outcome. Our study, the first to compare post-treatment with conventional baseline tumor signatures, suggests post-treatment signatures to offer additional predictive and prognostic information. These findings are logical given that tumor exposure to drug and resultant responses would shape outcome, and are clinically relevant as the majority of breast cancers are treated with systemic therapy.

The use of high throughput gene array allows simultaneous analysis of thousands of markers that take into account interactions between different genes and pathways, yielding composite predictive panels that could outperform clinical predictors and prognosticators^{10,12}. We found both post-treatment and relative change signatures to be more predictive than baseline signatures, independent of clinical or pathological response to chemotherapy, suggesting that they are not merely surrogates of treatment response. While unchallenged tumor signatures offer information on inherent tumor biology, including aggressiveness and intrinsic drug sensitivity, treatment-induced signatures provide information on tumor biology that has been modified by drug exposure, including acquired resistance, and have the potential to uncover pathways to target resistant tumors¹³.

If the post-treatment tumor signatures were to become gold standard in predicting outcome, what is the best timing to obtain the sample? Interestingly, our signatures, derived at the 3-week time point, were applicable to signatures obtained at the 16-week time point¹⁴. A study on breast tumor biomarkers after 2 and 12 weeks of neoadjuvant endocrine therapy suggested that significant changes occur by 2 weeks^{15,16}. Delaying post-treatment biopsy till after 12-16 weeks of chemotherapy may render most tumors too small for biopsy and/or too necrotic for gene expression analysis¹⁷. Obtaining a biopsy within 24-48 hours after chemotherapy could provide information on immediate tumor reactions to drug,^{18,19} while a later biopsy at 2-3 weeks provides information on subacute changes related to repair and immune responses, which may be valuable in predicting short- and mid-term treatment-related outcomes.

How can these results be realistically applied in practice? While post-treatment biopsy in patients with clinical T3-4 tumors on neoadjuvant chemotherapy is feasible, the majority of patients with clinical T1-3 breast cancers undergo definitive surgery before chemotherapy. If these results are validated, a paradigm shift may be considered to treat most patients with chemotherapy prior to surgery, using post-treatment gene expression data to identify poorer-risk patients who require change in their neoadjuvant regimen or additional adjuvant therapy to improve outcome.

Our study is the first to compare pre- and post-treatment gene signatures in predicting treatment outcome, and the largest of such studies with paired samples. We derived and tested three panels of signatures (T1, T2, TΔ) using the same datasets and methodologies to reduce biases and improve validity. In addition, the data was validated using two independent datasets. The drug-induced changes were due to a single drug, providing valuable drug-specific information that could not be obtained from signatures developed from drug combinations^{7,8}. A limitation of our study is the small sample size. While the results are convincing, validation in larger cohorts is clearly desired. Secondly, while the post-treatment signatures were specific to a single drug for each patient, the predictive panels were obtained for patients who received either adriamycin or docetaxel, since treatment outcome was due to both drugs. Nonetheless, our signatures were applicable to two independent cohorts treated with single agent adriamycin or docetaxel respectively, suggesting general cellular response rather than drug-specific changes to chemotherapy. It was particularly noteworthy that our signatures successfully distinguished two prognostic groups in an independent dataset with expression analysis performed on a different platform with only 3440 overlapping genes. We expect drug- or regimen-specific signatures to be even more informative, and such studies are warranted. Thirdly, our pathological response definition is not the conventional pathological complete response rate associated with long-term outcome²⁰, although our division correlated significantly with PFS in a larger cohort, suggesting clinical relevance.

Drug-induced gene expression signatures may be more informative than unchallenged signatures in predicting treatment outcomes. These findings have important clinical implications, and may be further exploited to uncover pathways as therapeutic targets in resistant tumors.

### Example 2

### Peripheral Blood Genomic Markers Whose Expression Levels Reflect that of Breast Tumor Genomic Markers Predict Drug Treatment and Sensitivity

Repeated tumor sampling during treatment may not be feasible for many patients. We therefore studied peripheral mononuclear cell (PBMC) gene expression levels in an attempt to identify peripheral blood markers that may serve as more accessible surrogates to predict drug treatment and response.

Our objectives were to (1) compare expression level changes between PBMC and tumor probe sets in response to chemotherapy and (2) identify PBMC probe sets that are concordant with tumor probe sets in discriminating doxorubicin and docetaxel treatment and between sensitive and resistant tumor to either drug.

### Methods

Chemonaive breast cancer patients were treated with an alternating sequential regimen of doxorubicin (75 mg/m² 3 weekly) and docetaxel (75 mg/m² 3 weekly) and randomized to start with either drug. RNA from primary tumor before and after 3 weeks, and from PBMC before and 24 hours after the first cycle of each drug, was hybridized on the Affymetrix HG-U133+2 array containing 54,675 probe sets.

Median expression change ratios between tumor and PBMC in response to chemotherapy were compared. PBMC probe sets concordant with tumor probes sets that discriminated between doxorubicin and docetaxel treatment and between sensitive and resistant tumors to either drug were identified.

Validation was conducted on an independent data set. Training was conducted on paired PBMC samples in the original cohort using a binary regression model.

### Results

Pre- and post-treatment tumors from 47 patients were studied, including 35 with paired PBMC samples. 230 pre- and 85 post-treatment PBMC probe sets showed strong correlation in expression level (Pearson correlation coefficient >0.9) with the corresponding pre- and post-treatment tumor probe set, and included genes involved in transcription regulation and binding.

Of the 975 tumor probe sets whose changes distinguished doxorubicin from docetaxel treatment, 9 corresponding PBMC probe sets were identified (p<0.001) whose changes at 24 hours distinguished the two treatments with 89% accuracy.

Of the 1081 and 1526 tumor probe sets whose changes predicted treatment response to doxorubicin and docetaxel, 19 and 15 informative PBMC probe sets predicted response to each drug with 100% and 80% accuracy, and included TNF receptor protein 1 and trefoil factor 1 for doxorubicin- and folliculin and dynein for docetaxel-response. When the PBMC probe sets were validated in an independent test set treated with docetaxel (n=21), 86% was correctly classified as docetaxel-treated, while 83% of responders were correctly classified.

In conclusion, peripheral blood contains genomic markers whose expression levels closely reflect that of breast tumor markers, and are promising as surrogates to predict drug treatment and sensitivity.

### REFERENCES

1. Perou, C. M. et al. Molecular portraits of human breast tumours. Nature 406, 747-52 (2000).

2. van de Vijver, M. J. et al. A gene-expression signature as a predictor of survival in breast cancer. N Engl J Med 347, 1999-2009 (2002).

3. Fan, C. et al. Concordance among gene-expression-based predictors for breast cancer. N Engl J Med 355, 560-9 (2006).

4. Chang, H. Y. et al. Robustness, scalability, and integration of a wound-response gene expression signature in predicting breast cancer survival. Proc Natl Acad Sci U S A 102, 3738-43 (2005).

5. Paik, S. et al. A multigene assay to predict recurrence of tamoxifen-treated, node-negative breast cancer. N Engl J Med 351, 2817-26 (2004).

6. Chang, J. C. et al. Gene expression profiling for the prediction of therapeutic response to docetaxel in patients with breast cancer. Lancet 362, 362-9 (2003).

7. Ayers, M. et al. Gene expression profiles predict complete pathologic response to neoadjuvant paclitaxel and fluorouracil, doxorubicin, and cyclophosphamide chemotherapy in breast cancer. J Clin Oncol 22, 2284-93 (2004).

8. Thuerigen, O. et al. Gene expression signature predicting pathologic complete response with gemcitabine, epirubicin, and docetaxel in primary breast cancer. J Clin Oncol 24, 1839-45 (2006).

9. Iwao-Koizumi, K. et al. Prediction of docetaxel response in human breast cancer by gene expression profiling. J Clin Oncol 23, 422-31 (2005).

10. Bonnefoi, H. et al. Validation of gene signatures that predict the response of breast cancer to neoadjuvant chemotherapy: a substudy of the EORTC 10994/BIG 00-01 clinical trial. Lancet Oncol 8, 1071-8 (2007).

11. Cheok, M. H. et al. Treatment-specific changes in gene expression discriminate in vivo drug response in human leukemia cells. Nat Genet 34, 85-90 (2003).

12. van 't Veer, L. J. et al. Gene expression profiling predicts clinical outcome of breast cancer. Nature 415, 530-6 (2002).

13. Bild, A. H. et al. Oncogenic pathway signatures in human cancers as a guide to targeted therapies. Nature 439, 353-7 (2006).

14. Sorlie, T. et al. Gene expression profiles do not consistently predict the clinical treatment response in locally advanced breast cancer. Mol Cancer Ther 5, 2914-8 (2006).

15. Dowsett, M. et al. Biomarker changes during neoadjuvant anastrozole, tamoxifen, or the combination: influence of hormonal status and HER-2 in breast cancer—a study from the IMPACT trialists. J Clin Oncol 23, 2477-92 (2005).

16. Dowsett, M. et al. Short-term changes in Ki-67 during neoadjuvant treatment of primary breast cancer with anastrozole or tamoxifen alone or combined correlate with recurrence-free survival. Clin Cancer Res 11, 951s-8s (2005).

17. Chang, J. C. et al. Patterns of resistance and incomplete response to docetaxel by gene expression profiling in breast cancer patients. J Clin Oncol 23, 1169-77 (2005).

18. Buchholz, T. A. et al. Global gene expression changes during neoadjuvant chemotherapy for human breast cancer. Cancer J 8, 461-8 (2002).

19. Modlich, O., Prisack, H. B., Munnes, M., Audretsch, W. & Bojar, H. Immediate gene expression changes after the first course of neoadjuvant chemotherapy in patients with primary breast cancer disease. Clin Cancer Res 10, 6418-31 (2004).

20. Bear, H. D. et al. Sequential preoperative or postoperative docetaxel added to preoperative doxorubicin plus cyclophosphamide for operable breast cancer: National Surgical Adjuvant Breast and Bowel Project Protocol B-27. J Clin Oncol 24, 2019-27 (2006).

21. Kaplan, E. L. & Meier, P. (1958). “Nonparametric estimation from incomplete observations”. Journal of the American Statistical Association 53: 457-481.

1. A method of selecting a treatment for further treatment of a tumor that has been exposed to chemotherapy, the method comprising:
(i) performing gene expression analysis on a sample obtained from a patient having a tumor that has been exposed to chemotherapy so as to obtain a chemotherapy gene expression data set;
(ii) analysing said chemotherapy gene expression data set to predict response of the tumor to the chemotherapy, said analysing comprising comparing gene expression data from the chemotherapy gene expression data set with gene expression data from a further chemotherapy gene expression data set obtained by performing gene expression analysis on a further sample obtained from the patient following further exposure of the tumor to the same or different chemotherapy so as to determine a change in gene expression of the tumor, and analysing the change in gene expression to predict response of the tumor to the chemotherapy;
(iii) based on said prediction selecting a treatment for further treatment of said tumor.

2. The method of claim 1 wherein the step of analysing said chemotherapy gene expression data set comprises comparing gene expression data from said chemotherapy gene expression data set with gene expression data obtained for a corresponding tumor type which has been exposed to a corresponding chemotherapy and which gene expression data has been related with the response of the tumor type to the chemotherapy.

3. The method of claim 2 wherein a positive relationship of gene expression data between said gene expression data from said chemotherapy gene expression data set and gene expression data obtained for a corresponding tumor type is predictive of the response of the tumor being treated to the existing chemotherapy.

4. The method of claim 1 wherein the step of analysing said chemotherapy gene expression data set comprises comparing gene expression data from said chemotherapy gene expression data set with gene expression data from corresponding tumor types known to be poor responders to the same chemotherapy.

5. The method of claim 4 wherein positive relationship of gene expression data from said chemotherapy gene expression data set with gene expression data from corresponding tumor types known to be poor responders to the same chemotherapy is predictive of poor response of the tumor to the chemotherapy.

6. The method of claim 1 wherein the step of analysing said chemotherapy gene expression data set comprises comparing gene expression data from said chemotherapy gene expression data set with gene expression data from corresponding tumor types known to be good responders to the same chemotherapy.

7. The method of claim 6 wherein positive relationship of gene expression data from said chemotherapy gene expression data set with gene expression data from corresponding tumor types known to be good responders to the same chemotherapy is predictive of good response of the tumor to the chemotherapy.

8. A method of selecting a treatment for further treatment of a tumor that has been exposed to chemotherapy, the method comprising:
I. performing gene expression analysis on a sample obtained from a patient having a tumor that has been exposed to chemotherapy so as to obtain a first chemotherapy gene expression data set;
II. comparing gene expression data from said first chemotherapy gene expression data set with gene expression data from a second chemotherapy gene expression data set obtained by performing gene expression analysis on a sample obtained from a patient having a tumor after further exposure of the tumor to the chemotherapy so as to determine a change in gene expression;
III. analysing the change in gene expression to predict response of the tumor to the chemotherapy;
IV. based on said prediction selecting a treatment for further treatment of said tumor.

9. The method of claim 8 wherein the step of analysing the change in gene expression to predict response of the tumor to the chemotherapy comprises comparing change(s) in gene expression from II. with change(s) in first and second chemotherapy gene expression data sets obtained for a corresponding tumor type which has been exposed to a corresponding chemotherapy and which changes have been related with the response of the tumor type to the chemotherapy.

10. The method of claim 8 wherein a positive relationship of change(s) between said change in gene expression from II. and change(s) in first and second chemotherapy gene expression data sets obtained for a corresponding tumor type is predictive of the response of the tumor being treated to the existing chemotherapy.

11. The method of claim 8 wherein the step of analysing the change in gene expression to predict response of the tumor to the chemotherapy comprises comparing change(s) in gene expression from II. with change(s) in first and second chemotherapy gene expression data sets from corresponding tumor types known to be poor responders to the same chemotherapy.

12. The method of claim 11 wherein positive relationship of change(s) in gene expression from II. with change(s) in first and second chemotherapy gene expression data sets from corresponding tumor types known to be poor responders to the same chemotherapy is predictive of poor response of the tumor to the chemotherapy.

13. The method of claim 8 wherein the step of analysing the change in gene expression to predict response of the tumor to the chemotherapy comprises comparing change(s) in gene expression from II. with change(s) in first and second chemotherapy gene expression data sets from corresponding tumor types known to be good responders to the same chemotherapy.

14. The method of claim 13 wherein positive relationship of change(s) in gene expression from II with change(s) in first and second chemotherapy gene expression data sets from corresponding tumor types known to be good responders to the same chemotherapy is predictive of good response of the tumor to the chemotherapy.

15. The method of claim 1 wherein the step of selecting a treatment for further treatment of said tumor comprises selection of one or more of:
maintenance of the existing chemotherapy or treatment regime;
discontinuing the existing chemotherapy or treatment regime;
starting a new chemotherapy or treatment regime;
maintenance of the existing chemotherapy but altering the existing treatment regime.

16. The method of claim 1 wherein on the basis of a prediction of poor response to chemotherapy the step of selecting a treatment for further treatment of said tumor comprises one or more of:
discontinuing the existing chemotherapy or treatment regime;
starting a new chemotherapy or treatment regime;
maintenance of the existing chemotherapy but altering the existing treatment regime.

17. The method of claim 1 wherein on the basis of a prediction of good response to chemotherapy the step of selecting a treatment for further treatment of said tumor comprises one or more of:
maintenance of the existing chemotherapy or treatment regime;
maintenance of the existing chemotherapy but altering the existing treatment regime.

18. The method of claim 1 wherein one or more of the sample(s) on which gene expression analysis is performed is taken from tumor tissue in the patient.

19. The method of claim 1 wherein one or more of the sample(s) on which gene expression analysis is performed is taken from a bodily fluid of the patient.

20. A method of predicting response of a tumor to chemotherapy, comprising:
I. performing gene expression analysis on a sample obtained from a patient having a tumor that has been exposed to chemotherapy so as to obtain a first chemotherapy gene expression data set;
II. comparing gene expression data from said first chemotherapy gene expression data set with gene expression data from a second chemotherapy gene expression data set obtained by performing gene expression analysis on a sample obtained from the patient after further exposure of the tumor to the chemotherapy so as to determine a change in gene expression;
III. analysing the change in gene expression to predict response of the tumor to the chemotherapy, said analysis comprising comparing change(s) in gene expression from II. with change(s) in first and second chemotherapy gene expression data sets obtained for a corresponding tumor type which has been exposed to a corresponding chemotherapy and which changes have been related with the response of the tumor type to the chemotherapy.

21. The method of claim 20 wherein one or more of the sample(s) on which gene expression analysis is performed is taken from tumor tissue in the patient.

22. The method of claim 20 wherein one or more of the sample(s) on which gene expression analysis is performed is taken from a bodily fluid of the patient.

23. The method of claim 8 wherein the step of selecting a treatment for further treatment of said tumor comprises selection of one or more of:
maintenance of the existing chemotherapy or treatment regime;
discontinuing the existing chemotherapy or treatment regime;
starting a new chemotherapy or treatment regime;
maintenance of the existing chemotherapy but altering the existing treatment regime.

24. The method of claim 8 wherein on the basis of a prediction of poor response to chemotherapy the step of selecting a treatment for further treatment of said tumor comprises one or more of:
discontinuing the existing chemotherapy or treatment regime;
starting a new chemotherapy or treatment regime;
maintenance of the existing chemotherapy but altering the existing treatment regime.

25. The method of claim 8 wherein on the basis of a prediction of good response to chemotherapy the step of selecting a treatment for further treatment of said tumor comprises one or more of:
maintenance of the existing chemotherapy or treatment regime;
maintenance of the existing chemotherapy but altering the existing treatment regime.

26. The method of claim 8 wherein one or more of the sample(s) on which gene expression analysis is performed is taken from tumor tissue in the patient.

27. The method of claim 8 wherein one or more of the sample(s) on which gene expression analysis is performed is taken from a bodily fluid of the patient.
